(19) United States
(12) Patent Application Publication
(10) Pub. No.: US 2010/0331200 A1
(43) Pub. Date: Dec. 30, 2010

(54) POST TRANSLATIONAL MODIFICATION PATTERN ANALYSIS

(76) Inventors: Neal F. Gordon, Lexington, MA (US); James R. Graham, Arlington, MA (US); Timothy Kenneth Nadler, Framingham, MA (US)

Correspondence Address: MILLIPORE CORPORATION, 290 CONCORD ROAD, BILLERICA, MA, 01821, US

(21) Appl. No.: 12/295,239

(22) PCT Filed: Mar. 30, 2007
(86) PCT No.: PCT/US07/07947

§ 371 Date: Apr. 27, 2009

Related U.S. Application Data
(60) Provisional application No. 60/788,341, filed on Mar. 31, 2006.

(51) Int. Cl. C40B 30/04; G01N 33/53; C40B 40/00

(52) U.S. Cl. 506/9; 436/501; 506/13; 435/7.1

(57) ABSTRACT

This invention relates to methods and apparatus for detecting the pattern of post translational modification in a protein or in a plurality of proteins in a sample. One or more target proteins are subjected to predetermined proteolysis to yield plural peptide fragments comprising potential post translational modification sites. The fragments and the state of such sites are analyzed to yield a post translational pattern for the protein or proteins.

### CROSS-REFERENCE TO RELATED APPLICATIONS

The present application claims the benefit of and priority to U.S. provisional patent application Ser. No. 60/788,341, filed Mar. 31, 2006, the entire disclosure of which is incorporated by reference herein for all purposes.

### FIELD OF THE INVENTION

This invention relates to methods and apparatus for detecting the pattern of post translational modification in a protein or in a plurality of proteins in a sample. One or more target proteins are subjected to predetermined proteolysis to yield plural peptide fragments comprising potential post translational modification sites. The fragments and the state of such sites are analyzed to yield a post translational pattern for the protein or proteins.

### BACKGROUND

Post-translational protein modification is a ubiquitous mechanism in living systems, for example, as a signaling device. For example, kinase-mediated phosphorylation of various proteins enables signaling pathways important to intracellular communication. Receptor tyrosine kinases (RTKs) are a prototypical example of a class of proteins that undergo this post-translational modification. Specifically, RTKs are a subclass of cell-surface receptors with intrinsic, ligand-controlled tyrosine kinase activity. These receptors regulate diverse functions in normal cells and have a crucial role in oncogenesis. RTKs are activated via two processes: enhancement of the receptor's catalytic activity and the creation of binding sites to recruit downstream signaling proteins. Both of these processes are usually accomplished by receptor autophosphorylation on tyrosine residues, due to ligand-mediated oligomerization. Auto-phosphorylation of tyrosine residues throughout the cytosolic domain generates docking sites for modular domains that recognize phosphotyrosines in a sequence-specific context. Two such domains are the SH2 domain and the phosphotyrosine-binding (PTB) domain.

Docking proteins can modulate the activity of the receptor in a variety of ways, including removal or addition of phospho-groups from tyrosines and through the phosphorylation and dephosphorylation of other non-tyrosine residues. Additionally, docking proteins can function as bridges, allowing other proteins to bind and become activated, leading to a variety of downstream events. Accordingly, the dynamic phosphorylation state of the receptor, at the multitude of its phosphorylation sites, is of significant research interest as it may elucidate what potential effects the receptor can mediate and which signaling pathways can be activated.

Analysis and measurement of post translational modifications using mass spectrometry (MS) based techniques has been and continues to be a powerful discovery-based platform that is able to discriminate modification events to specific sites of proteins. However, these MS-based approaches are poorly suited for routine measurement due to a number of factors including: a) low sample throughput where measurement of a single sample can take several weeks to complete, b) inherent difficulties in providing a quantitative measurement, c) high capital cost for equipment, d) need for highly trained operators, and e) need for tedious and specially designed sample preparation techniques to reduce the complexity of the mixture.

Antibody-based methods to measure protein modifications events are powerful, used extensively in research applications, and largely overcome many of the limitations of MS-based approaches. A large number of site specific antibodies, for example site specific anti-phospho-antibodies, are available and most commonly used in Western blots, which provide a qualitative assessment of the modification state. However, the quantitative determination of the modification state, for example the phosphorylation state, of a protein (such as a receptor) is complicated by a number of factors, including the lack of comprehensive antibody reagents, lack of appropriate specificity of the measurement, inability to differentiate one site of a type of modification from another, and absence of appropriate calibration standards.

Thus there is a need to develop improved methods for post translational modification pattern analysis within a single protein or within a plurality of proteins.

### SUMMARY OF THE INVENTION

The present invention is directed to methods, apparatus, and reagents for reproducible determination of the pattern of post translational modification, for example the phosphorylation pattern, of one or a plurality of target proteins, including parallel determination of the post translational modifications of the target proteins in a complex biological sample. By “pattern of post translational modification” is meant the differential post translational states at one or more types of post translational modification sites on a protein or on a plurality of proteins.

Practice of the invention enables effective and reliable detection and quantitation of the post translational modification pattern of one or multiple proteins in the same solution, as they existed at the time of sampling (for example, at the time a cell is lysed), exploiting, for example, optical or other automated detection methods.

Practice of the invention also enables tracking the changes in such post translational modification patterns, e.g., phosphorylation patterns over time, across samples, or in reaction to one or more external influences such as contacting a candidate compound in a drug screening or a pharmaceutical composition in research, or upon exposure to temperature/light/other forms of energy field changes, etc.

“Pattern” as used herein goes beyond a mere fingerprint of which proteins in the cell are post translational modified, but rather relates to the pattern of post translational modification, for example phosphorylation, on a single protein, e.g., the ability to discriminate among the multiple phosphorylation sites on a single protein. When referring to the post translational modification pattern of multiple target proteins within a sample, “pattern” also includes the collective pattern of those belonging to the individual target proteins of interest, or a subset of such target proteins.

In certain embodiments, the target proteins are within the same or related signal transduction pathways or biological pathways. In other embodiments, at least two of the target proteins may be unrelated to each other, or may belong to different branch pathways downstream of a common upstream activator.

In certain embodiments, the target proteins are known to be positively or negatively associated with certain disease or pathological states, such as various cancers, obesity, cardiovascular disease, etc.; or certain developmental or physiological conditions, such as pregnancy, etc.

The invention exploits the use of targeted protein fragmentation protocols, the general nature of which are disclosed in detail in co-pending U.S. application Ser. No. 10/712,425, filed Nov. 13, 2003, and now published as US-20040180380-A1.

For use in this invention, the fragmentation protocols are designed to digest a potentially or differentially post translationally modified target protein, and typically at least several of these in parallel, to produce two or more, typically many, proteolytic digestion fragments of the respective target protein or proteins. At least some of these fragments comprise one or more potential post translational modification sites, for example potential phosphorylation sites such as serine, threonine, and most notably tyrosine residues, and also comprise a sequence of amino acids which permits their detection within complex protein mixtures.

Detection is accomplished using antibodies or other binding agents which recognize the respective epitopes defined by the sequences. The method exploits immobilized binding agents designed specifically to detect and to capture a plurality of fragments from one or from a plurality of target proteins in parallel and simultaneously. Next, the bound peptide fragments are contacted with one or a set of labeled reagents, typically optically labeled reagents, which bind with one or more post translational modification sites, for example phosphorylated tyrosines, threonines, or serines, to indicate which of the peptide fragments have been post translationally modified. The binding agents can be immobilized in an array at known positions to permit determination of which peptides are post translationally modified.

The presence and/or amount of potential post translationally modified sites may be further compared to the total amount of post translationally modified peptide fragments to determine the degree of translational modification on the target protein or proteins.

Although a given peptide may theoretically contain more than one potential post translationally modified site, the use of different fragmentation methods may yield overlapping (but not identical) peptides encompassing such potential post translationally modified sites. Results obtained from different sets of fragmentation schemes may be combined to yield information as precise as the post translationally modified state of individual amino acids on the target protein or proteins.

Thus, the method produces data indicative of which amino acids or fragments of which specific protein(s) are or are not post translationally modified, and (optionally) if so, to what extent/degree (such as % post translationally modification). The data is sufficient to permit determination of the post translational modification pattern of respective target proteins, precise to the level of individual post translational modification sites or fragments.

Post translational modifications for which patterns can be determined include, but are not limited to, acetylation, amidation, deamidation, prenylation (such as farnesylation or geranylation), formylation, glycosylation, hydroxylation, methylation, myristoylation, phosphorylation, ubiquitination, SUMOylation, NEDDylation, ribosylation and sulphation.

Accordingly, in one aspect, the invention provides a method for determining a pattern of post translational modification of a target protein. First, the target protein is digested using a predetermined proteolysis protocol to produce plural peptide fragments of the target protein, at least a portion of which comprises one or a plurality of sites for post translational modification and present an epitope capturable by a binding agent. Next, the plural peptide fragments are contacted with immobilized binding agents which bind respectively to the epitopes and leave exposed a product of post translational modification thereby to capture plural said respective peptide fragments. Then, the captured respective peptide fragments are contacted with one or a plurality of labeled reagents which bind specifically to the exposed product of the post translational modification to discern which of the peptide fragments have been post translationally modified. Schematic drawings of the subject method are shown in FIGS. 1 and 2.

In certain embodiments, the target protein is from the cytosol or plasma membrane of a cell, such as a transmembrane receptor (e.g., RTK).

In certain embodiments, the method of the invention may be used for more than one target protein, which may be involved in the same or different signaling pathway.

In certain embodiments, the method comprises the steps of first digesting the protein in the sample using a predetermined proteolysis protocol designed specifically to produce plural peptide fragments of the target protein having certain properties. Namely, the proteolysis protocol results in the generation of fragments comprising amino acid sequences which comprise one or a plurality of potentially post translationally modified sites, and which present an epitope which is unambiguously indicative of the presence of the peptide fragment in the sample (known as “peptide epitope tags” or “PETs”). These peptide fragments are contacted with immobilized binding agents which bind respectively to the epitopes so as to capture the fragments and to permit determination of the presence or absence of different fragments in the sample.

Next, the bound peptide fragments are contacted with one or more reagents which bind with one or more potential post translationally modified sites to discern the post translational modification state of the respective fragments.

In certain embodiments, the binding agents are immobilized in an array. In certain embodiments, the reagent advantageously is optically labeled, although many other known or hereafter developed label forms may be used. In certain embodiments, the method may comprise detecting optical signals generated by the optical labels on a reagent bound to a respective peptide fragment captured at one or more selected positions on an array so that a pattern of signals on the array corresponds to a pattern of post translational modification on one or multiple said target proteins.

In certain embodiments in which the post translational modification is phosphorylation, the one or a plurality of labeled reagents comprise a phospho-binding domain from a macromolecule, a phospho-binding protein domain, an antibody or a part thereof that recognizes an epitope comprising a phosphate group, a phospho-binding aptamer domain or a portion thereof, a catalytically inert kinase construct or a part thereof which binds a phosphate group, or a catalytically inert phosphatase enzyme or a part thereof having phosphate binding activity.

In certain embodiments, the flexibility of the method may be enhanced by contacting the bound peptide fragments with a set of labeled reagents, e.g., reagents having different binding properties so as to permit discrimination among differently forms of post translational modification, or different fragments of the same target proteins. For example, the reagent can optionally specifically bind phosphorylated or unphosphorylated tyrosines, serines and/or threonines.

In certain embodiments, the sample may comprise other proteins in addition to the one or more target proteins, e.g., a cell lysate or body fluid such as serum, and does not require any protein pre-fractionation or separation prior to analysis.

In certain embodiments, the method of the invention comprises multiplexed detection of plural different target proteins in a sample comprising a mixture of proteins. In this case the predetermined proteolysis protocol is designed to produce plural peptide fragments permitting resolution of different target proteins within the sample using techniques as disclosed herein.

In an embodiment, at least 3, 5, 10, 20, 50, 100, 200, 500, or 1000 different target proteins or modification sites are represented in one array. Related target proteins or modification sites may be physically close to one another on the array.

In an embodiment, target proteins belonging to similar signaling pathways of different organisms are represented on the array. In an embodiment, target proteins belonging to signaling pathways of at least two different organisms are represented. In an embodiment, all capture agents are specific for target proteins belonging to the same signal transduction pathway wherein all target proteins of the signal transduction pathway that are predictably post translationally modified are represented.

In an embodiment, one or more of the key target proteins are post translationally modified upon activation or inhibition of at least two of the signal transduction pathways. In this embodiment, the status of post translational modification of these key proteins may indicate cross-talk between different, or even seemingly irrelevant, signaling pathways, since signals converge to these key proteins from many different pathways.

In an embodiment, the array has a recovery rate of at least 50 percent. In an embodiment, the array has an overall positive predictive value for occurrence of proteins in said sample of at least 90 percent. In an embodiment, the array comprises at least 1,000 or 10,000 different capture agents bound to the support. In an embodiment, the addressable array is collection a of beads, each of which comprises a discrete species of capture agent and one or more labels which identify the bead.

In another preferred form, the method comprises quantitating the binding of the respective peptide fragments to the binding agents, or quantitating the binding of the reagent(s) to the captured respective peptide fragments to determine at least the relative quantity of different post translationally modified groups in different positions on the target protein(s), or the relative quantity of different differentially post translationally modified said target protein(s) in the sample. For example, an RTK may have two or more differentially phosphorylation isoforms, each phosphorylated at a different (e.g., not completely overlapping) set of phospho-amino acids, or both phosphorylated at the same set of phospho-amino acids but phosphorylated to a different extent in at least one of the phospho-amino acids. For example, isoform 1 can be phosphorylated at residue 1 (e.g., a Tyr), but not residue 2 (e.g., a Ser); while isoform 2 can be phosphorylated at residue 2, but not residue 1. If anti-p-Tyr Ab is used as the detection reagent, only isoform 1 will be detected. If anti-p-Ser Ab is used as the detection reagent, only isoform 2 will be detected. The ratio of the different isoforms can then be assessed based on the two measurements.

In certain embodiments, the method may, and typically does, include a post analysis step of computationally deconvoluting data indicative of which of the fragments are modified and which are not to determine the state of modification of one or more target proteins. The computation may be done manually, or may be aided by computer algorithms or software.

In another aspect, the invention provides an article of manufacture for determining a pattern of post translational modification on one or a plurality of target proteins in a sample. The apparatus comprises a set of immobilized capture agents, individual ones of which bind to peptide fragments generated by a predetermined digestion protocol applied to the one or plurality of target proteins. The generated respective peptide fragments each comprise an amino acid sequence or set of sequences which, when bound to a capture agent or agents, is an indication of the presence of the peptide fragment in the sample, comprise one or a plurality of sites for post translational modification, and expose while bound to its capture agent a product of a post translational modification, if present on the fragment.

In certain embodiments, the article of manufacture may further comprise one or a plurality of detectably labeled reagent(s) which bind specifically with the product of post translational modification exposed on the captured peptide fragments. Optionally, the bound reagent reveals the pattern of post translational modification on respective individual target proteins by deconvoluting the pattern of signals from the reagent labels bound to peptide fragments in turn bound to the set of immobilized capture agents. Optionally, the article of manufacture may further comprise apparatus or reagents for digesting said mixture of proteins in said sample reliably to produce said peptide fragments.

In various embodiments, the reagent which binds specifically with the product of post translational modification may bind with a product of post translational modification selected from the group consisting of acetylation, amidation, deamidation, prenylation (such as farnesylation or geranylation), formylation, glycosylation, hydroxylation, methylation, myristoylation, phosphorylation, ubiquitination, SUMOylation, NEDDylation, ribosylation and sulphation. The reagent which binds specifically with the product of post translational modification may bind with one or more types of modification, for example, phosphorylated tyrosines, phosphorylated threonines, and/or phosphorylated serines, to reveal the phosphorylation pattern of respective individual said target proteins.

In certain embodiments, the capture agents are immobilized on a solid surface in an array. In certain embodiments, the detectably labeled reagent or reagents preferably comprise optically detectable labels. Optionally, the capture agents are immobilized on a solid surface at known positions in an array.

In certain embodiments, the article may further comprise a written protocol specifying directions for digesting the sample comprising the target protein(s), and also apparatus or reagents for digesting the mixture of proteins so as to permit a user reliably to produce the fragments. Certain embodiments may include comprising a readable (e.g., computer readable) protocol specifying directions for digesting said target protein reliably to produce said peptide fragments.

In certain embodiments, the article is designed to obtain the pattern of post translational modification of a plurality of target proteins in parallel, the apparatus further comprising a set of immobilized capture agents, individual ones of which bind to peptide fragments generated by a predetermined digestion protocol applied to the plurality of different target proteins, which generated fragments each respectively comprise an amino acid sequence or sequences which, when bound to a said capture agent or agents, is an indication of the presence of the peptide fragment in the sample, and permits resolution of different the target proteins in the sample.

Another aspect of the invention provides a method of comparing a pattern of phosphorylation or other post translational modification of a target protein in plural cellular samples. First, a first pattern of post translational modification of the target protein in a cellular sample is obtained under a first condition using the method described above. Next, a second pattern of post translational modification of the target protein in a cellular sample is obtained under a second condition. Then, the changes are compared, if any, in the pattern of post translational modification of the target protein between cells from the first and the second conditions. The patterns can be, for example, phosphorylation patterns.

In certain embodiments, the first condition represents a first time point of the cellular sample, and the second condition represents a second, later time point of the cellular sample. This can be used, for example, to track the dynamic change of phosphorylation states of a target protein(s) in a sample over time.

Similar methods may also be used to track the dynamic change of phosphorylation states of a target protein(s) in a sample before and after a stimulation.

In certain embodiments, the stimulation may be treatment of cells by a growth factor, a cytokine, a hormone, a steroid, a lipid, an antigen, a small molecule (Ca²⁺, cAMP, cGMP), an osmotic shock, a heat or cold shock, a pH change, a change in ionic strength, a mechanical force, a viral or bacterial infection, or an attachment or detachment from a neighboring cell or a surface with or without a coated protein, etc.

In certain embodiments, the first condition of the cellular sample is before a stimulus and the second condition of the cellular sample represents the sample after the stimulus, for example, before and after contact with a compound or exposure of the cells to a drug or drug candidate. For example, the method of the invention may be used for drug screening, if the readout is a change of phosphorylation state of a certain target protein(s).

In certain embodiments, the first condition represents a healthy tissue, and the second condition represents a disease tissue of the same kind as the healthy tissue. This can be used, for example, to diagnose the presence, progress, and/or predict the prognosis of certain diseases or physiological conditions where the phosphorylation pattern of certain target proteins are indicative of disease status.

It should be understood that different embodiments of the invention, including those described under different aspects of the invention, are meant to be generally applicable to all aspects of the invention. Any embodiment may be combined with any other embodiment unless inappropriate. All examples are illustrative and non-limiting.

### BRIEF DESCRIPTION OF THE DRAWINGS

FIG. 1 is a schematic representation of the Epidermal Growth Factor Receptor (EGFR) with known and suspected sites of serine, threonine, and tyrosine (indicated by thick black lines to the right of the receptor), and Lys-C fragments containing sites of phosphorylation (bracketed with thin black lines within the receptor).

FIG. 2 is a schematic representation of the phospho-containing Lys-C fragments from the cytosolic domain of the EGFR. Antibodies raised to EGFR fragment-specific sequences are used to capture the phospho-fragments from a Lys-C digested sample. Single or multiple detection reagents are then added, which recognize specific phosphorylation sites or recognize all phosphorylation sites. These detection reagents may be anti-phospho antibodies, phospho-binding protein domains (for example, SH2 domains), a catalytically inert, but still capable of binding phosphate groups, phosphatase enzyme or parts thereof, or some other chemical means of detecting the presence of phosphorylation.

FIG. 3 is a schematic representation of the detection of a post translational modification of a modifying protein, for example, ubiquitin, small ubiquitin-like modifier protein (SUMO), or NEDD8, covalently attached to a target protein lysine residue (SEQ ID NO: 1). The attachment protects against Lys-C digestion at that particular lysine residue such that, following digestion with Lys-C, two covalently attached peptide fragments are generated (SEQ ID NOs: 2-3). In a sandwich assay, one binding agent, for example a capture antibody, can be directed against the fragment on the target protein and a second binding agent, for example a single detection antibody, can be directed against the fragment on the covalently attached peptide fragment of the modifying protein to indicate the presence of the post translational modification.

FIG. 4 is a schematic representation of the detection of a post translational modification of a chain of modifying proteins, for example, ubiquitins or SUMOs, covalently attached to a target protein lysine residue (SEQ ID NO: 1). A second or subsequent modifying protein (SEQ ID NO: 3) is further covalently attached at a lysine residue of a first or previous modifying protein (SEQ ID NO: 4). The attachments at the lysine residues protect against Lys-C digestion at those particular lysine residues. Following digestion with Lys-C, the target protein and the second modifying protein are fragmented (SEQ ID NOs: 2-3), but the first modifying protein was protected. Two binding agents, for example two detection antibodies, directed against portions of a first modifying protein indicate that a second modifying protein was present to inhibit the Lys-C digestion at the lysine residue on the first or previous modifying protein.

FIGS. 5A and 5B show Western blots of undigested cell lysates following cellular exposure to EGF. The presence of phosphorylation, measured using anti-phosphotyrosine (4G10) antibody (FIG. 5A) and anti-phosphoERK1/2 antibody (FIG. 5B), was observed at 0.5 minutes following exposure of the cells to EGF.

FIGS. 6A-6C show standard curves generated from synthetic peptides for total EGFR (FIG. 6A), an EGFR peptide fragment comprising tyrosine residues 1069/1092 (FIG. 6B), and an EGFR peptide fragment comprising tyrosine residues 1110/1125 (FIG. 6C).

FIG. 7 shows results from a multiplexed antibody array identifying protein fragments with phosphorylated tyrosine residues. The protein fragments were generated from cells exposed to EGF for the time periods shown in the figure. Samples for the array were prepared in triplicate.

FIG. 8 shows the total EGFR concentration at various timepoints following cellular stimulation with 50 μg/mL EGF.

FIG. 9 shows the concentration of phosphorylated EGFR tyrosine residues 1069/1092 and tyrosine residues 1110/1125, respectively, in response to EGF stimulation for the time periods shown in the figure.

FIG. 10 expresses the concentration of phosphorylated EGFR tyrosine residues 1069/1092 and tyrosine residues 1110/1125, respectively, as a percent of total EGFR concentration, in response to EGF stimulation for the time periods shown in the figure.

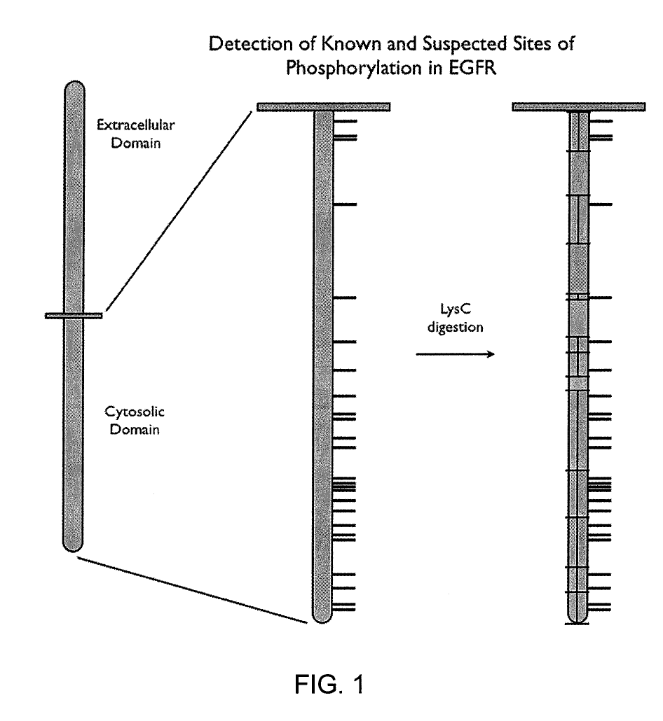

### DETAILED DESCRIPTION

### 1. Overview

In general, this invention relates to methods and apparatus for detecting the pattern of post translational modification on individual target proteins, for example phosphorylation on tyrosine, serine, and/or threonine. The target proteins may be involved in signaling, whose function is mediated in part by the combination of kinase-induced phosphorylation and phosphatase-induced de-phosphorylation, e.g. receptors such as RTKs and other downstream effector molecules. The invention provides methods/apparatus to detect and/or measure the extent of post translational modification at various sites on proteins or on a plurality of proteins (e.g., phosphorylation) to follow the dynamics of post translational modifications at a site-specific level.

According to the invention described herein, a binding agent-based (e.g., antibody-based) measurement system that overcomes the limitations described above has now been developed. The method comprises a novel application and adaptation of the technology disclosed in co-pending published US application publication number US-20040180380-A1.

In a general sense, the detection of the post translational modification (e.g., phosphorylation) states and the measurement of the degree of post translational modification (e.g., phosphorylation) are simplified, by fragmenting the target proteins into peptide fragments, such that groups of post translational modification (e.g., phosphorylation) sites fall on separate proteolytic fragments. Although the description below uses one of the most common post translational modifications—phosphorylation—as an example, the scope of the invention should be understood to encompass all post translational modifications unless specified otherwise.

The subject methods/apparatus allows for the simultaneous measurement of multiple modification events, e.g., the detection and measurement of several separate entities, rather than the accumulative effect of multiple modification events on a single entity.

According to the invention described herein, the sample is optionally pretreated to provide better digestion results. Possible treatments include sample denaturation (by heat, and/or chemical reagents such as 6-8 M guanidine HCl or urea or SDS, etc.). See, for example, US 2005-0069911 A1.

Using the PET (peptide epitope tag) technology, highly specific binding agents, such as various forms of antibodies can be generated that are able to unambiguously distinguish among the different fragments. The selection of PETs is also described in detail in US 2005-0069911 A1. Other details and the various aspects of the PET technology are described in co-pending U.S. patent application publications US 2004-0029292 A1, US 2004-0038307 A1, US 2004-0180380 A1, US 2005-0069911 A1, US 2006-0014212 A1, and/or US 2005-0255491 A1, EP 1320754.

A set of binding agents (e.g., antibodies) can be used in an array format to capture simultaneously a collection of peptide fragments that contain at least one site that is potentially post translationally modified as a consequence of the cellular state at the time of sampling. In certain embodiments, the PET that is bound by the binding agent does not contain any potential modification sites, and the binding of the binding agent to the PET is not substantially affected by the modification state of the fragment containing the PET. In other embodiments, the PET may itself contain one or more modification site(s), and only the modified PET is bound by the binding agent. In yet another embodiment, the peptide fragment may contain more than one PETs, wherein at least one PET is not sensitive to the modification state of the peptide fragment, while at least another PET is sensitive to the modification state of the peptide fragment.

The amount of modification on each fragment may be determined in a “sandwich” assay format using a second reagent that is labeled. The second reagent can be a pan detection reagent (e.g. anti-phospho-Tyr), a site specific detection reagent, or somewhere in between, such as an SH2 domain that recognizes a common p-Tyrosine motif.

The assay readout may be further normalized relative to response for a synthetic peptide standard representing each post translationally modified site. In addition, the measurement of total receptor concentration is accomplished using the standard PET sandwich assay format such that modification measurements can be normalized to the amount of receptor present in the sample.

The invention provides: (1) a quantitative measure of post translational modification, (2) the simultaneous, individual measurement of modification at each modification site of an individual protein, and/or (3) the ability to measure quantitatively the receptor protein itself Such quantitative measurement is very difficult using conventional antibody technology due to difficulties in providing an appropriate calibration standard for this class of proteins (membrane associated proteins). The invention also provides (4) normalization of assay results to the level of total receptor protein in each sample. The combination of these four attributes provide the basis for determining the activation state of a protein. Important biological applications are provided in examples below.

The invention provides an easy yet comprehensive means to precisely map the post translational modification pattern of any and all individual potential modification sites (i.e., amino acid residues) within any given target protein, any combination or subsets of such sites, or any groups of related or unrelated target proteins. For each individual site, the method of the invention provides qualitative as well as quantitative measure of modification states, including the degree of modification at each site.

The powerful array format also allows multiplex, simultaneous, and individual measurements of modification at each modification site.

The methods of the invention applies to all types of proteins and post translational modifications. In various embodiments, the target protein or proteins is/are phosphoproteins, and the post translational modification is phosphorylation. Its advantage over the conventional antibody technology is many fold, especially for applications requiring quantitative measurement of certain membrane receptor proteins, such as RTKs. This task is generally very difficult when performed using conventional antibody technologies, due large part to difficulties in providing an appropriate calibration standard for this class of proteins, e.g., membrane associated proteins. The instant invention not only provides suitable ways to solubilize and prepare membrane protein samples, but also provides standardized measurement conditions for qualitative and quantitative measurement of post translational modification states at sites within proteins.

The methods can be used for pattern analysis of virtually all kinds of post translational modifications, including but not limited to phosphorylation, glycosylation, ubiquitination, SUMOylation, NEDDylation, etc., as long as the modifications can be reliably detected, for example, by a reagent that can either specifically bind to the post translational modification, or an intermediate moiety that directly and specifically binds to the post translational modification. The method also applies to the detection of alternative splicing forms otherwise identical proteins.

The methods include combining various patterns of post-translation modifications, in combination, to yield a comprehensive pattern of post translational modification of one or a plurality of proteins. Other modified forms of proteins, such as alternative splicing forms, can be included in such a comprehensive pattern analysis.

The results of the subject method may be presented in a number of ways. In various embodiments, the results are normalized to the level of total receptor protein in each sample, such that the degree of phosphorylation between different sites within a target protein, or between the same phosphorylation site within different samples, may be directly compared.

### 2. Definitions

As used herein, the term “PET” (peptide epitope tag) or unique recognition sequence is intended to mean an amino acid sequence that, when detected in a particular sample, unambiguously indicates the presence of a type of protein, a protein, or a portion thereof, from which the PET was derived. For example, a PET can be selected such that its presence in a sample, as indicated by detection of an authentic binding event with a capture agent designed to selectively bind with the sequence, necessarily means that the protein which comprises the sequence is present in the sample. A PET can be derived from a surface region of a protein, a buried region, a splice junction, or post translationally modified region. However, following denaturation and/or fragmentation of a protein mixture in a sample, a useful PET must present a binding surface that is solvent accessible, and must bind with significant specificity to a selected capture agent with minimal cross reactivity.

A PET will preferably not have any closely related sequence, such as determined by a nearest neighbor analysis, among the other types of proteins, proteins, or portions thereof (depending on the investigation) that may be present in the sample. In certain embodiments, a PET is present within the protein from which it is derived and in no other protein that may be present in the sample, cell type, or species under investigation. In certain embodiments, a PET is present within a portion of a protein and in no other portion of that protein, and optionally, in no other protein present in the sample. In the context of protein isoforms, such as phosphorylation isoforms, or alternative splicing isoforms, degradation product of the same protein, or certain disease genes encoding different length of protein products, such as in the Huntington disease (HD) protein, a PET may be shared by the isoforms. Thus, the specificity of a PET depends on the investigation. By way of examples, a PET might be specific for a protein type in a proteome or in a sample; specific for a protein in a proteome, among protein types, or in a sample; or specific for a protein fragment in a proteome, among protein types, within a protein, or in a sample.

Moreover, in certain embodiments, such as in a sandwich immunoassay, where two epitopes recognized by two capture agents (respectively) reside on the same peptide fragment, it may be the combination of two epitopes that is unique to the protein type, the protein, or the protein fragment in a sample, even when each epitope alone might not strictly qualify as a PET (because it is present on other protein types, proteins, or fragments in the sample). For example, phosphoprotein 1 may have epitope A in close proximity to a tyrosine residue, phosphoprotein 2 may have epitope A in close proximity to a serine residue, and phosphoprotein 3 may have epitope A in close proximity to a threonine residue. When all three phosphoproteins are present in a sample, epitope A does not qualify as a “PET” insofar as it does not unambiguously indicate the presence of any of phosphoproteins 1, 2, or 3. However, the combination of epitope A and the epitope comprising the phosphotyrosine, phosphoserine, or phosphothreonine residue can uniquely identify each phosphoprotein.

In certain embodiments, such epitopes can be used in a sandwich immunoassay to generate the phosphorylation pattern within one or more proteins in a sample. For example, after predetermined proteolysis, phosphoprotein 1 may yield three protein fragments each comprising a potential phosphorylation site. Fragment 1 may comprise epitope A and a tyrosine residue, fragment 2 may comprise epitope A and a serine residue, and fragment 3 may comprise epitope B and a tyrosine residue. In this example, the phosphorylation pattern of each phosphoprotein can be identified by the unique combinations of the epitope and phosphoepitope on each fragment, even though each epitope and phosphoepitope appears more than once in the protein.

This concept of a combination-PET is not limited to phosphorylation site discrimination. Any two epitopes that do not qualify as PETs alone (e.g., because each sequence is shared among different, maybe unrelated proteins in the sample) may represent a unique combination that is not shared by any other protein. The use of combination-PETs, in conjunction with the sandwich assays, is a powerful approach for post translational modification pattern analysis that has distinct advantage over single capture agent assays.

Most frequently, a PET is referred to as being a peptide sequence that is present in only one protein type, protein, or fragment in the proteome of a species. Such a PET useful in a human sample may in fact be present within the structure of proteins of other organisms. Similarly, a PET useful in an adult cell sample is “unique” to that sample even though it may be present in the structure of other different proteins of the same organism at other times in its life, such as during embryogenesis, or is present in other tissues or cell types different from the sample under investigation. Depending on the investigation conducted, such a single PET may be “unique” even though the same amino acid sequence is present in the sample from a different protein type, protein, or fragment, provided, for example, one or more of its amino acids are derivatized, and a binder can be developed which resolves the derivatized form or, for example, the PET is part of a combination-PET as described above.

When referring herein to “uniqueness” with respect to a PET, the reference is always made in relation to the foregoing. Thus, within the human genome, a PET may be an amino acid sequence that is truly unique to the protein type, protein, or fragment from which it is derived. Alternatively, it may be unique just to the sample from which it is derived, but the same amino acid sequence may be present in, for example, the murine genome. For example, when referring to a sample which may contain proteins from multiple different organisms, uniqueness refers to the ability to unambiguously identify and discriminate between proteins from the different organisms, such as being from a host or from a pathogen.

Thus, a PET may be present within more than one protein type, protein, or fragment in the species, provided it is unique to the sample from which it is derived. For example, a PET may be an amino acid sequence (or combination of sequences for combination PETs) that is unique to: a certain cell type, e.g., a liver, brain, heart, kidney or muscle cell; a certain biological sample, e.g., a plasma, urine, amniotic fluid, genital fluid, marrow, spinal fluid, or pericardial fluid sample; a certain biological pathway, e.g., a G-protein coupled receptor signaling pathway or a tumor necrosis factor (TNF) signaling pathway, or, for discrimination between protein fragments, a certain protein or group of proteins.

In this sense, the invention provides a method to identify application-specific PETs, depending on the type of proteins present in a given sample. This information may be readily obtained from a variety of sources. For example, when the whole genome of an organism is concerned, the sequenced genome provides each and every protein sequences that can be encoded by this genome, sometimes even including hypothetical proteins. This “virtually translated proteome” obtained from the sequenced genome is expected to be the most comprehensive in terms of representing all proteins in the sample. Alternatively, the type of transcribed mRNA species (“virtually translated transcriptome”) within a sample may also provide useful information as to what type of proteins may be present within the sample. The mRNA species present may be identified by DNA microarrays, SNP analysis, or any other suitable RNA analysis tools available in the art of molecular biology. An added advantage of RNA analysis is that it may also provide information such as alternative splicing and mutations. Finally, direct protein analysis using techniques such as mass spectrometry may help to identify the presence of specific post-translation modifications and mutations, which may aid the design of specific PETs for specific applications. For example, WO 03/001879 A2 describes methods for determining the phosphorylation status or sulfation state of a polypeptide or a cell using mass spectrometry, especially ICP-MS. In a related aspect, mass spectrometry, when coupled with separation techniques such as 2-D electrophoresis, GC/LC, etc., has provided a wealth of information regarding the profile of expressed proteins in specific samples.

For instance, Pieper et al. (Proteomics 3: 1345-1364, 2003) exemplifies a typical approach for MS-based protein profiling study. In a typical such study, proteins from a specific sample are first separated using a chosen appropriate method (such as 2-DE). To identify a separated protein, a gel spot or band is cut out, and in-gel tryptic digestion is performed thereafter. The gel must be stained with a mass spectrometry-compatible stain, for example colloidal Commassie Brilliant Blue R-250 or Farmer's silver stain. The tryptic digest is then analyzed by MS such as MALDI-MS. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS is typically used to increase the likelihood of getting a database match. The PMF only contains the masses of the peptides. CID-MS/MS (collision induced dissociation of tandem MS) of peptides gives a spectrum of fragment ions that contain information about the amino-acid sequence. Adding this information to the peptide mass fingerprint allows Mascot to increase the statistical significance of a match. It is also possible in some cases to identify a protein by submitting only the raw MS/MS spectrum of a single peptide, a so-called MS/MS Ion Search, such is the amount of information contained in these spectra. MS/MS of peptides in a PMF can also greatly increase the confidence of a protein identification, sometimes giving very high Mowse scores, especially with spectra from a TOF/TOF™.

Applied Biosystems 4700 Proteomics Analyzer, a MALDI-TOF/TOF™ tandem mass spectrometer, is unrivalled for the identification of proteins from tryptic digests, because of its sensitivity and speed. High-speed batch data acquisition is coupled to automated database searching using a locally-running copy of the Mascot search engine. When proteins cannot be identified by peptide mass mapping unambiguously, the digest can be further analyzed by a hybrid nanospray/ESI-Quadrupole-TOF-MS and MS/MS in a QSTAR mass spectrometer (Applied Biosystems Inc., Foster City, Calif.) for de novo peptide sequencing, sequence tag search, and/or MS/MS ion search. The static nanospray MS/MS is especially useful used when the target protein is not known (database absent). Applied Biosystems QSTAR® Pulsar i tandem mass spectrometer with a Dionex UltiMate capillary nanoLC system can be used for ES-LC-MS and MDLC (Multi-Dimensional Liquid Chromatography) analysis of peptide mixtures. A combination of these instruments can also perform MALDI-MS/MS, MDLC-ES-MS/MS, LC-MALDI, and Gel-C-MS/MS. With the Probot™ micro-fraction collector, HPLC can be interfaced with MALDI and spot peptides eluting from the nanoLC directly onto a MALDI target plate. This new LC-MALDI workflow for proteomics allows maximal potential for detecting proteins in complex mixtures by complementing the conventional 2-DE-based approach. For the traditional 2-DE approach, instruments such as the Bio-Rad Protean 6-gel 2-DE apparatus and Packard MultiProbe II-EX robotic sample handler, in conjunction with the Applied Biosystems 4700 Proteomics Analyzer, allow higher sample throughputs for complete proteome characterizations.

Studies such as this, using equivalent instruments described above, have accumulated a large amount of MS data regarding expressed proteins and their specific protease digestion fragments, mostly tryptic fragment, stored in the form of many MS databases. See, for example, MSDB (a non-identical protein sequence database maintained by the Proteomics Department at the Hammersmith Campus of Imperial College London. MSDB is designed specifically for mass spectrometry applications). PET analysis can be done on these tryptic peptides to identify PETs, which in turn is used for PET-specific antibody generation. The advantage of this approach is that it is known for sure that the (tryptic) peptide fragments will be generated in the sample of interest.

PETs identified based on the different methods described above may be combined. For example, in certain embodiments of the invention, multiple PETs need to be identified for any given target protein. Some of the PETs may be identified from sequenced genome data, while others may be identified from tryptic peptide databases.

The PET may be found in the native protein from which it is derived as a contiguous or as a non-contiguous amino acid sequence. It typically will comprise a portion of the sequence of a larger peptide or protein, recognizable by a capture agent either on the surface of an intact or partially degraded or digested protein, or on a fragment of the protein produced by a predetermined fragmentation protocol. The PET may be 5, 6, 7, 8, 9, 10, 11, 12, 13,14, 15, 16, 17, 18, 19 or 20 amino acid residues in length. In a preferred embodiment, the PET is 6, 7, 8, 9 or 10 amino acid residues, preferably 8 amino acids in length.

The term “discriminate”, as in “capture agents able to discriminate between”, refers to a relative difference in the binding of a capture agent to its intended protein analyte and background binding to other proteins (or compounds) present in the sample. In particular, a capture agent can discriminate between two different species of proteins (or species of modifications) if the difference in binding constants is such that a statistically significant difference in binding is produced under the assay protocols and detection sensitivities. In preferred embodiments, the capture agent will have a discriminating index (D.I.) of at least 0.5, and even more preferably at least 0.1, 0.001, or even 0.0001, wherein D.I. is defined as K_{d}(a)/K_{d}(b), K_{d}(a) being the dissociation constant for the intended analyte, K_{d}(b) is the dissociation constant for any other protein (or modified form as the case may be) present in sample.

As used herein, the term “capture agent” includes any agent which is capable of binding to a protein that includes a unique recognition sequence, e.g., with at least detectable selectivity. A capture agent is capable of specifically interacting with (directly or indirectly), or binding to (directly or indirectly) a unique recognition sequence. The capture agent is preferably able to produce a signal that may be detected. In a preferred embodiment, the capture agent is an antibody or a fragment thereof, such as a single chain antibody, or a peptide selected from a displayed library. In other embodiments, the capture agent may be an artificial protein, an RNA or DNA aptamer, an allosteric ribozyme or a small molecule. In other embodiments, the capture agent may allow for electronic (e.g., computer-based or information-based) recognition of a unique recognition sequence. In one embodiment, the capture agent is an agent not naturally found in a cell.

As used herein, the term “globally detecting” includes detecting at least 40% of the proteins in the sample. In a preferred embodiment, the term “globally detecting” includes detecting at least 50%, 60%, 65%, 70%, 75%, 80%, 85%, 90%, 95% or 100% of the proteins in the sample. Ranges intermediate to the above recited values, e.g., 50%-70% or 75%-95%, are also intended to be part of this invention. For example, ranges using a combination of any of the above recited values as upper and/or lower limits are intended to be included.

As used herein, the term “proteome” refers to the complete set of chemically distinct proteins found in an organism.

As used herein, the term “organism” includes any living organism including animals, e.g., avians, insects, mammals such as humans, mice, rats, monkeys, or rabbits; microorganisms such as bacteria, yeast, and fungi, e.g., Escherichia coli, Campylobacter, Listeria, Legionella, Staphylo-coccus, Streptococcus, Salmonella, Bordatella, Pneumococcus, Rhizobium, Chlamydia, Rickettsia, Streptomyces, Mycoplasma, Helicobacter pylori, Chlamydia pneumoniae, Coxiella burnetii, Bacillus Anthracis, and Neisseria; protozoa, e.g., Trypanosoma brucei; viruses, e.g., human im-munodeficiency virus, rhinoviruses, rotavirus, influenza virus, Ebola virus, simian immunodeficiency virus, feline leukemia virus, respiratory syncytial virus, herpesvirus, pox virus, polio virus, parvoviruses, Kaposi's Sarcoma-Associated Herpesvirus (KSHV), adeno-associated virus (AAV), Sindbis virus, Lassa virus, West Nile virus, enteroviruses, such as 23 Coxsackie A viruses, 6 Coxsackie B viruses, and 28 echoviruses, Epstein-Barr virus, caliciviruses, astroviruses, and Norwalk virus; fungi, e.g., Rhizopus, neurospora, yeast, or puccinia; tapeworms, e.g., Echinococcus granulosus, E. multilocularis, E. vogeli and E. oligarthrus; and plants, e.g., Arabidopsis thaliana, rice, wheat, maize, tomato, alfalfa, oilseed rape, soybean, cotton, sunflower or canola.

As used herein, “sample” refers to anything which may contain a protein analyte. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). The sample may also be a mixture of target protein containing molecules prepared in vitro.

As used herein, “a comparable control sample” refers to a control sample that is only different in one or more defined aspects relative to a test sample, and the present methods, kits or arrays are used to identify the effects, if any, of these defined difference(s) between the test sample and the control sample, e.g., on the amounts and types of proteins expressed and/or on the protein modification profile. For example, the control biosample can be derived from physiological normal conditions and/or can be subjected to different physical, chemical, physiological or drug treatments, or can be derived from different biological stages, etc.

“Predictably result from a treatment” means that a peptide fragment can be reliably generated by certain treatments, such as site specific protease digestion or chemical fragmentation. Since the digestion sites are quite specific, the peptide fragment generated by specific treatments can be reliably predicted in silico.

Further details of the various aspects of the invention are described below.

### 3. Detection of Post Translational Modifications

The subject methods in general can be used to detect and/or measure various post translational modifications on specific residues or fragments of one or more individual target proteins within a sample, or plural such target proteins in combination. More than 100 different such modifications of amino acid residues are known, examples include but are not limited to acetylation, amidation, deamidation, prenylation (such as farnesylation or geranylation), formylation, glycosylation, hydroxylation, methylation, myristoylation, phosphorylation, ubiquitination, SUMOylation, NEDDylation, ribosylation and sulphation. Sequence analysis softwares which are capable of determining putative post translational modification in a given amino acid sequence include the NetPhos server which produces neural network predictions for serine, threonine and tyrosine phosphorylation sites in eukaryotic proteins (available through www.cbs.dtu dot dk/services/Net-Phos), GPI Modification Site Prediction (available through mendel.imp.univie.ac dot at/gpi) and the ExPASy proteomics server for total protein analysis (available through www.expasy dot ch/tools).

In certain embodiments, PET moieties are those lacking any post translational modification sites, since post translationally modified amino acid sequences may complicate sample preparation and/or interaction with a capture agent. Notwithstanding the above, capture agents that can discriminate between post translationally forms of a PET, which may indicate a biological activity of the polypeptide-of-interest, can be generated and used in the present invention.

A very common example of post translational modification is the phosphorylation of OH group of the amino acid side chain of a serine, a threonine, or a tyrosine group in a polypeptide. Depending on the polypeptide, this modification can increase or decrease its functional activity. In one embodiment, the subject invention provides an array of capture agents that are variegated so as to provide discriminatory binding and identification of various post translationally modified forms of one or more proteins. In a preferred alternative embodiment, the subject invention provides an array of capture agents that are variegated so as to provide specific binding to one or more PETs uniquely associated with a modification of interest, which modification itself can be readily detected and/or quantitated by additional agents, such as a labeled secondary antibody specifically recognizing the modification (e.g., a phosphor-tyrosine antibody).

In a general sense, the invention provides a general means to detect/quantitate protein modifications. “Modification” here refers generally to any kind of non-wildtype changes in amino acid sequence, including post translational modification, alternative splicing, polymorphism, insertion, deletion, point mutation, etc. To detect/quantitate a specific modification within a potential target protein present in a sample, the sequence of the target protein is first analyzed to identify potential modification sites (such as phosphorylation sites for a specific kinase). Next, a potential fragment of the target protein containing such modification site is identified. The fragment is specific for a selected method of treatment, such as tryptic digestion or digestion by another protease, or a mixture of proteases, or reliable chemical fragmentation. A PET within (and unique) to the modification site-containing fragment can then be identified using the method of the instant invention. Fragmentation using a combination of two or more methods is also contemplated. Absolute predictability of the fragment size is desired, but not necessary, as long as the fragment always contains the desired PET and the modification site.

Antibody or other capture agents specific for the identified PET is obtained. The capture agent is then used in a sandwich ELISA format to detect captured fragments containing the modification. The site of the PET is proximal to the post translational modification site(s). Thus a binding to the PET by a capture agent will not interfere with the binding of a detection agent specific for the modified residue.

For illustrative purpose only, the capture agents described below in various embodiments of the invention are antibodies specific for PETs. However, it should be understood that any capture agents described above can be used in each of the following embodiments.

A. Phosphorylation

In various embodiments, the post translational modification to be detected on one or a plurality of proteins using the methods of the invention is phosphorylation, for example, phosphorylation on Tyr, Ser, or Thr residues of proteins.

The reversible addition of phosphate groups (e.g., phosphorylation and de-phosphorylation) to proteins is important for the transmission of signals within eukaryotic cells and, as a result, protein phosphorylation and de-phosphorylation regulate many diverse cellular processes.

For example, receptor tyrosine kinases (RTKs) represent attractive drug targets for a variety of diseases, including cancer. Because RTKs tend to be structurally similar, drugs targeted against one receptor could potentially have off-target effects against other RTKs. These off-target effects could be benign or could lead to toxicities. Therefore, the ability to query the activation state of one or many RTKs would be greatly beneficial in determining drug specificity. Additionally, because certain downstream effector molecules may be used by multiple pathways which can involve feedback loops back to the receptor, drugs targeting these signaling molecules may also have off-target effects on the receptors which should be known as early as possible in the drug discovery and development process. Querying receptor activation, therefore, represents a high throughput method of screening drug compounds.

In another example, elucidating the function of individual proteins is an important goal in basic research towards a better understanding of biology. Common strategies to determine protein function involve evaluating cellular responses under a variety of conditions, including: (a) suppression of protein expression (e.g. RNAi), (b) outright gene knock-outs so that the protein is not expressed at all, (c) mutation of the protein, and (d) over expression of the protein. Biological systems are replete with examples of redundancy where cellular functions are regulated through a variety of complementary and competing pathways. Consequently, the impact of modifying protein expression or mutation of the protein often has unforeseen consequences. These consequences on signaling are likely to manifest in changes in receptor phosphorylation patterns. Querying receptor activation, therefore, represents a potentially basic strategy in combination with standard cell biology practices.

In yet another example, since defects or alterations in RTK activities do correlate with specific disease state, the tools to query the activation state of a large number of RTKs could produce significant diagnostic and prognostic benefits. Receptor activation fingerprints may be correlative with a particular disease and/or disease severity. Such fingerprints could then be used to personalize a treatment plan and to monitor the effectiveness of a given treatment.

To detect the presence and/or quantitate the amount of a phosphorylated peptide in a sample, anti-phospho-amino acid antibodies can be used to detect the presence of phospho-peptides. There are numerous commercially available phosphotyrosine specific antibodies that can be adapted to be used in the instant invention. Merely to illustrate, phosphotyrosine antibody (ab2287) [13F9] of Abcam Ltd (Cambridge, UK) is a mouse IgG1 isotype monoclonal antibody that reacts specifically with phosphotyrosine and shows minimal reactivity by ELISA and competitive ELISA with phosphoserine or phosphothreonine. The antibody reacts with free phosphotyrosine, phosphotyrosine conjugated to carriers such as thyroglobulin or BSA, and detects the presence of phosphotyrosine in proteins of both unstimulated and stimulated cell lysates.

Similarly, Research Diagnostics Inc. (Flanders, N.J.) provides a few similar anti-phosphotyrosine antibodies. Among them, RDI-PHOSTYRabmb is a mouse mIgG2b isotype monoclonal antibody reacts strongly and specifically with phosphotyrosine-containing proteins and can be blocked specifically with phosphotyrosine. No reaction with either phosphothreonine or phospho-serine is detected. This antibody appears to have broad cross-species reactivity, and is reactive with various tyrosine-phosphorylated proteins of human, chick, frog, rat, mouse and dog origin.

Research Diagnostics Inc. also provides phosphoserine-specific antibodies, such as RDI-PHOSSERabr, which is an affinity-purified rabbit antibody made against phosphoserine containing proteins. The antibody reacts specifically with serine phosphorylated proteins and shows no significant cross reactivity to other phosphothreonine or phosphotyrosine by western blot analysis. This antibody is suitable for ELISA according to the manufacture's suggestion. The company also provides a mouse IgG1 monoclonal anti-phosphoserine antibody RDI-PHOSSEabm, which reacts specifically with phosphorylated serine, both as free amino acid or conjugated to carriers as BSA or KLH. No cross reactivity is observed with non-phosphorylated serine, phosphothreonine, phosphotyrosine, AmpMP or ATP.

RDI-PHOSTHRabr is an affinity isolated rabbit anti-phosphothreonine antibody (anti-pT) provided by Research Diagnostics Inc. Both antigen-capture and antibody-capture ELISA indicated that the anti-phosphothreonine antibodies can recognize threonine-phosphorylated protein, phosphothreonine and lysine-phosphothreonine-glycine random polymer, respectively. Direct, competitive antigen-capture ELISA demonstrated that the antibodies are specifically inhibited by free phosphothreonine, phosvitin but not by free phosphoserine, phosphotyrosine, threonine and ATP. The company also provides a mouse IgG2b monoclonal anti-phospho-threonine antibody RDI-PHOSTHabm, which reacts specifically with phosphorylated threonine, both as free amino acid or conjugated to carriers as BSA or KLH. No cross reactivity is observed with non-phosphorylated threonine, phosphoserine, phosphotyrosine, AmpMP or ATP.

Molecular Probes/Invitrogen (Eugene, Oreg.) has developed a small molecule fluorophore phospho-sensor, referred to as Pro-Q Diamond dye, which is capable of ultrasensitive global detection and quantitation of phosphorylated amino acid residues in peptides and proteins displayed on micro-arrays. The utility of the fluorescent Pro-Q Diamond phosphosensor dye technology is demonstrated using phosphoproteins and phosphopeptides as well as with protein kinase reactions performed in miniaturized microarray assay format (Martin, et al., Proteomics 3: 1244-1255, 2003). Instead of applying a phosphoamino acid-selective antibody labeled with a fluorescent or enzymatic tag for detection, a small, fluorescent probe is employed as a universal sensor of phosphorylation status. The detection limit for phosphoproteins on a variety of different commercially available protein array substrates was found to be 312-625 fg, depending upon the number of phosphate residues. Characterization of the enzymatic phosphorylation of immobilized peptide targets with Pro-Q Diamond dye readily permits differentiation between specific and non-specific peptide labeling at picogram to subpicogram levels of detection sensitivity. Martin et al. (supra) also describe in detail the suitable protocols, instruments for using the Pro-Q stain, especially for peptides on microarrays.

One of the advantages of the method over other methods, such as identification of modified amino acids in proteins by mass spectrometry, is that the instant invention provides a much simpler technique that does not rely on expensive instruments, and thus can be easily adapted to be used in small or large laboratories, in industry or academic settings alike.

The determination of consensus phosphorylation site motifs by amino acid sequence alignment of known substrates has proven useful in this pursuit. These motifs can be helpful for predicting phosphorylation sites for specific protein kinases within a potential protein substrate. The table below summarizes merely some of the known data about specificity motifs for various well-studied protein kinases, along with examples of known phosphorylation sites in specific proteins (for a more extensive list, see Pearson, R. B., and Kemp, B. E. (1991). In T.

Hunter and B. M. Sefton (Eds.), Methods in Enzymology Vol. 200, pp. 62-81. San Diego: Academic Press). Phosphoacceptor residues are indicated in bold, amino acids which can function interchangeably at a particular residue are separated by a slash (/), and residues which do not appear to contribute strongly to recognition are indicated by an “X.” Some protein kinases such as CKI and GSK-3 contain phosphoamino acid residues in their recognition motifs, and have been termed “hierarchical” protein kinases (see Roach, J Biol. Chem. 266, 14139-14142, 1991 for review). They often require prior phosphorylation by another kinase at a residue in the vicinity of their own phosphorylation site. S(p) represents such preexisting phosphoserine residues.

TABLE 1
Exemplary data about specificity motifs for various well-studied protein kinases

| | Recognition | Phosphorylation | Protein Substrate |
|---|---|---|---|
| Protein Kinase | Motifs^{a} | Sites_{b} | (reference) |
| cAMP- | R-X-S/T^{c} | Y₇LRRASLAQLT | pyruvate kinase (2) |
| dependent | R-R/K-X-S/T | (SEQ ID NO: 5) | |
| Protein Kinase | | F₁RRLSIST | phosphorylase kinase, |
| (PKA, cAPK) | | (SEQ ID NO: 6) | a chain (2) |
| | | A₂₉GARRKASGPP | histon H1, bovine (2) |
| | | (SEQ ID NO: 7) | |
| Casein Kinase I | S(p)-X-X-S/T | R₄TLS(p)VSSLPGL | glycogen synthase, rabbit |
| (CKI, CK-1) | | (SEQ ID NO: 8) | muscle (4) |
| | | D₄₃IGS(p)ES(p)TEDQ | aₛ₁-casein (4) |
| | | (SEQ ID NO: 9) | |
| Casein Kinase | S/T-X-X-E | A₇₂DSESEDEED | PKA regulatory subunit, |
| II (CKII, CK-2) | | (SEQ ID NO: 10) | R_{II} (2) |
| | | L₃₇ESEEEGVPST | p34^{cdc2}, human (5) |
| | | (SEQ ID NO: 11) | |
| | | E₂₆DNSEDEISNL | acetyl-CoA carboxylase (2) |
| | | (SEQ ID NO: 12) | |
| Glycogen | S-X-X-X-S(p) | S₆₄₁VPPSPSLS(p) | glycogen syntase, human |
| Synthase | | (SEQ ID NO: 13) | (site 3b) (6, 2) |
| Kinase 3 | | S₆₄₁VPPS(p)PSLS(p) | glycogen syntase, human |
| (GSK-3) | | (SEQ ID NO: 14) | (site 3a) (6, 2) |
| Cdc2 Protein | S/T-P-X-R/K^{c} | P₁₃AKTPVK | histone H1, calf thymus (2) |
| Kinase | | (SEQ ID NO: 15) | |
| | | H₁₂₂STPPKKKRK | large T antigen (2) |
| | | (SEQ ID NO: 16) | |
| Calmodulin- | R-X-X-S/T | N₂YLRRRLSDSN | synapsin (site 1) (2) |
| dependent | R-X-X-S/T-V | (SEQ ID NO: 17) | |
| Protein Kinase | | K₁₉₁MARVFSVLR | calcineurin (2) |
| II (CaMK II) | | (SEQ ID NO: 18) | |
| Mitogen- | P-X-S/T-P^{d} | P₂₄₄LSP | c-Jun (7) |
| activated | X-X-S/T-P | (SEQ ID NO: 19) | |
| Protein Kinase | | P₉₂SSP | cyclin B (7) |
| (Extracellular | | (SEQ ID NO: 20) | |
| Signal-regulated | | V₄₂₀LSP | Elk-1 (7) |
| Kinase) | | (SEQ ID NO: 21) | |
| (MAPK, Erk) | | | |
| cGMP- | R/K-X-S/T | G₂₆KKKRKRSRKES | histon H2B (2) |
| dependent | R/K-X-X-S/T | (SEQ ID NO: 22) | |
| Protein Kinase | | F₁RRLSIST | phosphorylase kinase |
| (cGPK) | | (SEQ ID NO: 23) | (a chain) (2) |
| Phosphorylase | K/R-X-X-S-V/I | D₆QEKRKQISVRG | phosphorylase (2) |
| Kinase (PhK) | | (SEQ ID NO: 24) | |
| | | P₁LSRTLSVSS | glycogen synthase |
| | | (SEQ ID NO: 25) | (site 2) (2) |
| Protein Kinase | S/T-X-K/R | H₅₉₄EGTHSTKR | fibrinogen (2) |
| C (PKC) | K/R-X-X-S/T | (SEQ ID NO: 26) | |
| | K/R-X-S/T | P₁LSRTLSVSS | glycogen synthase |
| | | (SEQ ID NO: 27) | (site 2) (2) |
| | | Q₄KRPSQRSKYL | myelin basic protein (2) |
| | | (SEQ ID NO: 28) | |
| Ab1 Tyrosine | I/V/L-Y-X-X-P/F^{e} | | |
| Kinase | | | |
| Epidermal | E/D-Y-X | R₁₁₆₈ENAEYLRVAP | autophosphorylation (2) |
| Growth Factor | E/D-Y-I/L/V | (SEQ ID NO: 29) | |
| Receptor | | A₇₆₇EPDYGALYE | phospholipase C-g (2) |
| Kinase (EGF- | | (SEQ ID NO: 30) | |
| RK) | | | |

Single-letter Amino Acid Code: A = alanine, C = cysteine, D = aspartic acid, E = glutamic acid, F = phenylalanine, G = glycine, H = histidine, I = isoleucine, K = lysine, L = leucine, M = methionine, N = asparagine, P = proline, Q = glutamine, R = arginine, S = serine, T = threonine, W = tryptophan, V = valine, Y = tyrosine, X = any amino acid
^{a}Recognition motifs are taken from Pearson and Kemp (supra) except where noted. Consult Pearson and Kemp for a comprehensive list of phosphorylation site sequences and specificity motifs.
_{b}Subscr ipt numbers refer to first residue position within the given polypeptide chain.
^{c}From (1).
^{d}From (7).
^{e}From (8). See refs (8) and (9) for discussion of substrate recognition by Ab1.

### References Used in the Table Above:

1. Kennelly, P. J., and Krebs, E. G. (1991) J. Biol. Chem. 266, 15555-15558.
2. Pearson, R. B., and Kemp, B. E. (1991). In T. Hunter and B. M. Sefton (Eds.), Methods in Enzymology Vol. 200, (pp. 62-81). San Diego: Academic Press.
3. Roach, P. J. (1991) J. Biol. Chem. 266, 14139-14142.
4. Flotow, H. et al. (1990) J. Biol. Chem. 265, 14264-14269.
5. Russo, G. L. et al. (1992) J. Biol. Chem. 267, 20317-20325.
6. Fiol, C. J. et al. (1990) J. Biol. Chem. 265, 6061-6065.
7. Davis, R. J. (1993) J. Biol. Chem. 268, 14553-14556.
8. Songyang, Z. et al. (1995) Nature 373,536-539.
9. Geahlen, R. L. and Harrison, M. L. (1990). In B. E. Kemp (Ed.), Peptides and Protein Phosphorylation, (pp. 239-253). Boca Raton: CRC Press.

However, since the determinants of protein kinase specificity involve complex 3-dimensional interactions, these motifs, short amino-acid sequences describing the primary structure around the phosphoacceptor residue, are a significant oversimplification of the issue. They do not take into account possible secondary and tertiary structural elements, or determinants from other polypeptide chains or from distant locations within the same chain. Furthermore, not all of the residues described in a particular specificity motif may carry the same weight in determining recognition and phosphorylation by the kinase. In addition, the potential recognition sequence may be buried deep inside a tertiary structure of within a protein complex under physiological conditions and thus may never be accessible in vivo. As a consequence, they should be used with some caution. The instant invention provides a fast and convenient way to determine, on a proteome-wide basis, the identity of all potential kinase substrates that actually do become phosphorylated by the kinase of interest in vivo (or in vitro).

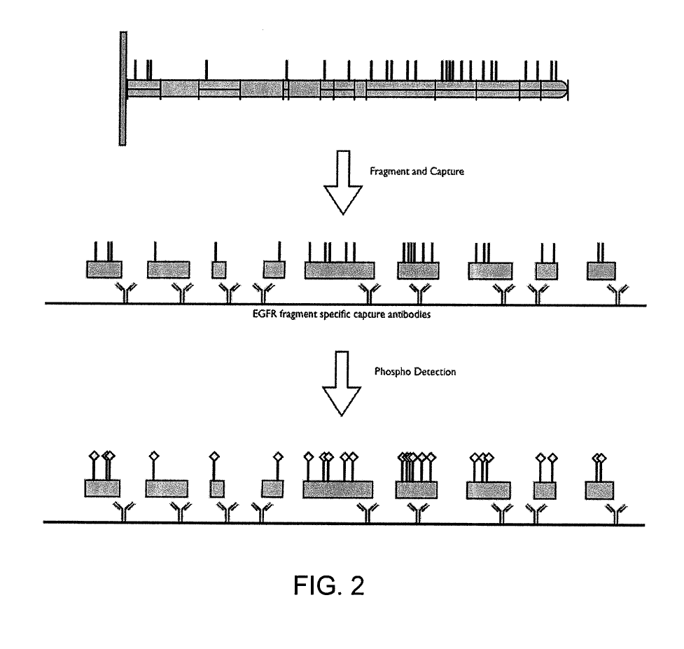

Specifically, consensus recognition sequences of a kinase (or a kinase subfamily sharing substrate specificity) can be identified based on, for example, the Pearson and Kemp or other kinase substrate motif database. For example, AKT (or PKB) kinase has a consensus phosphorylation site sequence of RXRXXS/T. All proteins in an organism (e.g., human) that contain such potential recognition sequences can be readily identified through routine sequence searches. Using the method of the instant invention, peptide fragments of these potential substrates, after a pre-determined treatment (such as trypsin digestion), which contain both the recognition motif and at least one PET can then be generated. Antibodies (or other capture agents) against each of these identified PETs can be raised and printed on an array to generate a so-called “kinase chip,” for example, an AKT chip. Using this chip, any sample to be studied can be treated as described above and then be incubated with the chip so that all potential recognition site-containing fragments are captured. The presence or absence of phosphorylation on any given “spot”—a specific potential substrate—can be detected/quantitated by, for example, labeled secondary antibodies (see FIG. 2). Thus, the identity of all AKT substrates in this organism under this condition may be identified in one experiment. The array can be reused for other samples by eluting the bound peptides on the array. Different arrays can be used in combination, preferably in the same experiment, to determine the substrates for multiple kinases.

The reversible phosphorylation of tyrosine residues is an important mechanism for modulating biological processes such as cellular signaling, differentiation, and growth, and if deregulated, can result in various types of cancer. Therefore, an understanding of these dynamic cellular processes at the molecular level requires the ability to assess changes in the sites of tyrosine phosphorylation across numerous proteins simultaneously as well as over time. Thus in another embodiment, the instant invention provides a method to identify the various signal transduction pathways activated after a specific treatment to a sample, such as before and after a specific growth factor or cytokine treatment to a sample cell. The same method can also be used to compare the status of signal transduction pathways in a diseased sample from a patient and a normal sample from the same patient.

Knowledge about the various signal transduction pathways existing in various organisms is accumulating at an astonishing pace. Science magazine's STKE (Signal Transduction Knowledge Environment) maintains a comprehensive and expanding list of known signal transduction pathways, their important components, relationship between the components (inhibit, stimulation, etc.), and cross-talk between key members of the different pathways. The “Connections Map” provides a dynamic graphical interface into a cellular signaling database, which currently covers at least the following broad pathways: immune pathways (IL-4, IL-13, Token-like receptor); seven-transmembrane receptor pathways (Adrenergic, PAC1 receptor, Dictyostelium discoideum cAMP Chemotaxis, Wnt/Ca²⁺/cyclic GMP, G Protein-Independent 7 Transmembrane Receptor); Circadian Rhythm pathway (murine and Drosophila); Insulin pathway; FAS pathway; TNF pathway; G-Protein Coupled Receptor pathways; Integrin pathways; Mitogen-Activated Protein Kinase Pathways (MAPK, JNK, p38); Estrogen Receptor Pathway; Phospho-inositide 3-Kinase Pathway; Transforming Growth Factor-β (TGF-β) Pathway; B Cell Antigen Receptor Pathway; Jak-STAT Pathway; STAT3 Pathway; T Cell Signal Transduction Pathway; Type 1 Interferon (α/β) Pathway; Jasmonate Biochemical Pathway; and Jasmonate Signaling Pathway. Many other well-known signal transduction pathways not yet included are described in detail in other scientific literature which can be readily identified in PubMed or other common search tools. Activation of most, if not all of these signal transduction pathways are generally characterized by changes in phosphorylation levels of one or more members of each pathway.

Thus in a general sense, the status of any given number of signaling pathways in a sample can be determined by taking a “snap shot” of the phosphorylation status of one or more key members of these selected pathways. For example, the Mitogen-activated protein (MAP)1 kinase pathways are evolutionarily conserved in eukaryotic cells. The pathways are essential for physiological processes, such as embryonic development and immune response, and regulate cell survival, apoptosis, proliferation, differentiation, and migration. In mammals, three major classes of MAP kinases (MAPKs) have been identified, which differ in their substrate specificity and regulation. These subgroups compose the extracellular signal-regulated kinases (ERKs), the c-Jun N-terminal kinases (JNKs), and the p38/RIC/CSBP kinases. ERKs are activated by a range of stimuli including growth factors, cell adhesion, tumor-promoting phorbol esters, and oncogenes, whereas JNK and p38 are preferentially activated by proinflammatory cytokines, and a variety of environmental stresses such as UV and osmotic stress. For this reason, the latter are classified as stress-activated protein kinases. Activation of the MAPKs is achieved by dual phosphorylation on threonine and tyrosine residues within a Thr-Xaa-Tyr motif located in the kinase subdomain VIII. This phosphorylation is mediated by a dual specificity protein kinase, MAPK kinase (MAPKK), and MAPKK is in turn activated by phosphorylation mediated by a serine/threonine protein kinase, MAPKK kinase. In addition to these activating kinases, several types of protein phosphatases have been also shown to control MAPK pathways by dephosphorylating the MAPKs or their upstream kinases. These protein phosphatases include tyrosine-specific phosphatases, serine/threonine-specific phosphatases, and dual specificity phosphatases (DSPs). Therefore, the activities of MAPKs can be regulated by upstream activating kinases and protein phosphatases, and the activation status can be determined by the phosphorylation status of, for example, ERK1/2, JNK, and p38.

Fragments of ERK1/2, JNK, and p38 containing the signature phosphorylation sites and PETs can be identified using the methods of the instant invention. Capture agents specifically recognizing such phosphorylation site-associated PETs can then be raised and immobilized on an array/chip. A sample (treated or untreated, thus containing high or low levels of phosphorylation of these pathway markers) can be digested and incubated with the chip, so as to determine the presence/absence of activation, degree, time course, duration of activation, etc.

In the same principal, target proteins representing many other related or perceived unrelated pathways may be manufactured on the same chip, since each pathway may be represented by from just one, to possibly all of the known pathway components. This type of chip may provide a comprehensive view of the various pathways that may be activated after a drug treatment. Pathway specific chips may also be used in conjunction to further determine the status of individual components within a pathway of interest.

Because of the important functions of the kinases in virtually all kinds of signal transduction pathways, it is not surprising to see that many drugs directly or indirectly affects phosphorylation status of various kinase substrates. Thus this type of array may also be used in drug target identification. Briefly, samples treated by different drug candidates may be incubated with the same kind of array to generate a series of activation profiles of certain chosen targets. These profiles may be compared, preferably automatically, to determine which drug candidate has the same or similar activation profile as that of the lead molecule.

This type of experiment will also yield useful information concerning candidate drugs selectivity, since it can be easily determined whether a candidate drug or drug analog actually has differential effects on various pathways, and if so, whether the difference is significant.

The same type of experiments can also be adapted to screen for drug candidates that lack undesired side effects or toxicity.

One aspect of this type of application also relates to the selection of specific protease(s) for fragmentation. The following table presents data resulting from analysis of protease sensitivity of potential phosphorylation sites in the human “kinome” (all kinases). This table may aid the selection of proteases among the several most frequently used proteases.

TABLE 2
Exemplary data useful for predetermined proteolysis

| | Total | | |
|---|---|---|---|
| | Peptide | Peptide Fragments with S/T/Y | |
| Enzymes | Fragments | =<10 aa | >10 aa |
| Chymotrypsin | 34,094 | 10930 (43%) | 14985 (57%) |
| S.A. V-8 E specific Enzyme | 34,233 | 6753 (32%) | 14917 (68%) |
| Post-Proline Cleaving Enzyme | 29,715 | 7077 (37%) | 12224 (63%) |
| Trypsin | 54,260 | 15,217 (53%) | 13311 (47%) |

B. Glycosylation

In certain embodiments, the post translational modification to be identified by the subject method is glycosylation.

A wide variety of eukaryotic membrane-bound and secreted proteins are glycosylated, e.g., they contain covalently-bound carbohydrate, and therefore are termed glycoproteins. In addition, certain intracellular eukaryotic proteins are also glycoproteins. Glycosylation of polypeptides in eukaryotes occurs principally in three ways (Parekh et al., Trends Biotechnol. 7: 117, 1989). Glycosylation through a glycosidic bond to an asparagine side-chain is known as N-glycosylation. Such asparagine residues only occur in the amino acid triplet sequence of Asn-Xaa-Ser/Thr, where Xaa can be any amino acid. The carbohydrate portion of a glycoprotein is also known as a glycan. O-glycans are linked to serine or threonine side-chains, through O-glycosidic bonds. In human, 284,535 octamer tags contains this NX(S/T) sequence, and 228,256 octamer PETs contains the NX(S/T) sequence. The latter is about 2.6% of the total octamer peptide tags in human. The N- and O-linked glycosylation are two of the most complex post translational modifications. The polypeptide may also be linked to a phosphatidylinositol lipid anchor through a carbohydrate “bridge”, the whole assembly being known as the glycosyl-phosphatidylinositol (GPI) anchor.

In recent years, the functional significance of the carbohydrate moieties has been increasingly appreciated (Rademacher et al., Ann. Rev. Biochem. 57: 785, 1988). Carbohydrates covalently attached to polypeptide chains can confer many functions to the glycoprotein, for example resistance to proteolytic degradation, the transduction of information between cells, and intercellular adhesion through ligand-receptor interactions (Gesundheit et al., J. Biol. Chem. 262: 5197, 1987; Ashwell & Harford, Ann. Rev. Biochem. 51: 531, 1982; Podskalny et al., J. Biol. Chem. 261: 14076, 1986; Dennis et al., Science 236: 582, 1987). As glycoforms are the product of a series of biochemical modifications, perturbations within a cell can have profound effects on their structure. With the increase in understanding of carbohydrate functions, the need for rapid, reliable and sensitive methods for carbohydrate detection and analysis has grown considerably.

Lectins are proteins that interact specifically and reversibly with certain sugar residues. Their specificity enables binding to polysaccharides and glycoproteins (even agglutination of erythrocytes and tumor cells). The binding reaction between a lectin and a specific sugar residue is analogous to the interaction between an antibody and an antigen. Substances bound to lectin may be resolved with a competitive binding substance or an ionic strength gradient. In addition, among other procedures, lectins can be labeled with biotin or digoxigenin, and subsequently detected by avidin-conjugated peroxidase or anti-digoxigenin antibodies coupled with alkaline phosphatase, respectively (Carlsson S R: Isolation and characterization of glycoproteins. In: Glycobiology. A Practical Approach. Fukuda M and Kobata A (eds). Oxford University Press, Oxford, pp 1-25, 1993.

For example, Concanavalin A (Con A) binds molecules that contain α-D-mannose, α-D-glucose and sterically related residues with available C-3, C-4, or C-5 hydroxyl groups. Like Con A, lentil lectin binds α-D-mannose, α-D-glucose, and sterically related residues, but lentil lectin distinguishes less sharply between glucosyl and mannosyl residues and binds simple sugars with lower affinity. Agarose wheat germ lectin specifically binds to N-acetyl-β-glucosaminyl residues. Wheat germ lectin specifically binds to N-acetyl-β-D-glucosaminyl residues. Psathyrella velutina lectin (PVL) preferentially interacts with the N-acetylglucosamine beta 1→2Man group. All these lectins can be used to detect the presence of various kinds of glycosylated peptides fragments after these PET-associated glycosylated fragments are captured from the sample by capture agents.

The GlycoTrack Kit from Glyko, Inc. (a Prozyme company, San Leandro, Calif.) detect glycosylation by using a specific carbohydrate oxidation reaction prior to binding of a high amplification color generating reagent. Briefly, a sample, either in solution or already immobilized to a support, is oxidized with periodate. This generates aldehyde groups that can react spontaneously with certain hydrazides at room temperature in aqueous conditions. Use of biotin-hydrazide following periodate oxidation leads to the incorporation of biotin into the carbohydrate (9). The biotinylated compound is detected by reaction with a streptavidin-alkaline phosphatase conjugate. Subsequently visualization is achieved using a substrate that reacts with the alkaline phosphatase bound to glycoproteins on the membrane, forming a colored precipitate.

Molecular Probes/Invitrogens (Eugene, Oreg.) offer a proprietary Pro-Q Emerald 300 fluorescent glycoprotein stain for detection of glycoproteins. The new Pro-Q Emerald 300 fluorescent glycoprotein stain reacts with periodate-oxidized carbohydrate groups, creating a bright green-fluorescent signal on glycoproteins. Depending upon the nature and the degree of glycosylation, this stain may be 50-fold more sensitive than the standard periodic acid—Schiff base method using acidic fuchsin dye. According to the manufacture, detection using the Pro-Q Emerald 300 glycoprotein stain is much easier than detection of glycoproteins using biotin hydrazide with streptavidin—horseradish peroxidase and ECL detection (Amersham Pharmacia Biotech). The stain can detect 50 ng of a typical glycosylated protein. Since the captured glycosylated PET-containing peptide fragments are much smaller than a typical peptide, as little as low nanogram to high picograms of captured peptides can be detected using this dye.

Thus to detect the presence and quantitation of glycosylation in a sample, all proteins or a subpopulation thereof which contains the potential glycosylation site NXS/T may be identified, and peptide fragments resulting from a specific pre-determined treatment may be analyzed to identify associated PETs. Capture agents against these PETs can then be raised. In a method analogous to the phosphorylation detection as described above, glycosylation can be detected/quantitated using the various detection methods

C. Modifying Protein Attachment—Ubiquitination, SUMOylation, NEDDylation

In certain embodiments, the post translational modification pattern to be identified on one or a plurality of target proteins involves the covalent attachment of an entire modifying protein to the one protein or plurality of target proteins. Post translational modifications that involve covalent attachment by an entire protein include ubiquitination, SUMOylation, and NEDDylation.

Ubiquitin is a small 76 amino acid protein that has a role in controlling stability, localization, and activity of the protein to which it binds. While the attachment of one ubiquitin (mono-ubiquitination) to a target protein may have signaling functions, the attachment of four or more ubiquitins (poly-ubiquitination) leads to protein degradation. Small ubiquitin-like modifier (SUMO) proteins are similar to ubiquitin except that they are not known to contribute to protein degradation. SUMOs have been shown to play roles in controlling signal transduction, transcription, nucleocytoplasmic transport, chromosome intregrity, and genomic stability, and they are believed to play a role in protein-protein interactions, for example, by facilitating the assembly of multi-protein complexes. SUMOs may also recruit regulatory factors and alter subcellular localization. NEDD8 is a ubiquitin-like, 81 amino acid, protein that similarly covalently attaches to a target protein. NEDDylation is similar to ubiquitination but uses a different system of enzymes in the attachment process.

Binding agents against each of these modifying proteins can be generated as described herein or obtained by commercial means. For example, there are at least 46 anti-ubiquitin commercial antibodies available from 14 different vendors. Cell Signaling Technology (Beverly, Mass.) offers mouse anti-ubiquitin monoclonal antibody, clone P4D1 (IgG1 isotype, Cat. No. 3936), which is specific for all species of ubiquitin, polyubiquitin, and ubiquitinated peptides.

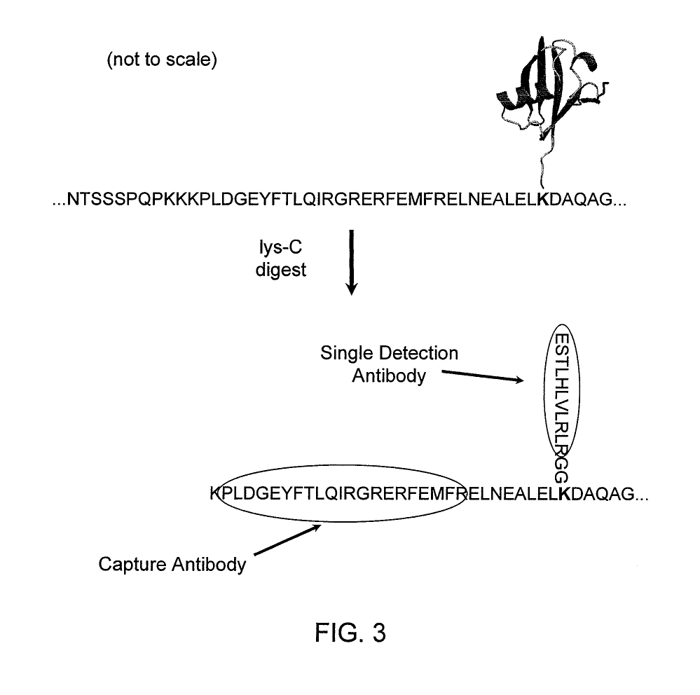

The covalent attachment of these modifying proteins typically occurs at specific lysine residues in the target protein and have been shown in MS experiments to prevent trypsin from cleaving those specific lysine residues, therefore, Lys-C would also be unable to cleave at this post translationally modified residue. Accordingly, Lys-C digestion of such a modified protein would result in the generation of two covalently attached peptide fragments, for which, in a sandwich assay, one binding agent could be directed against the fragment on the target protein and a second binding agent could be directed against a fragment on the covalently attached peptide fragment of the modifying protein. See FIG. 3.

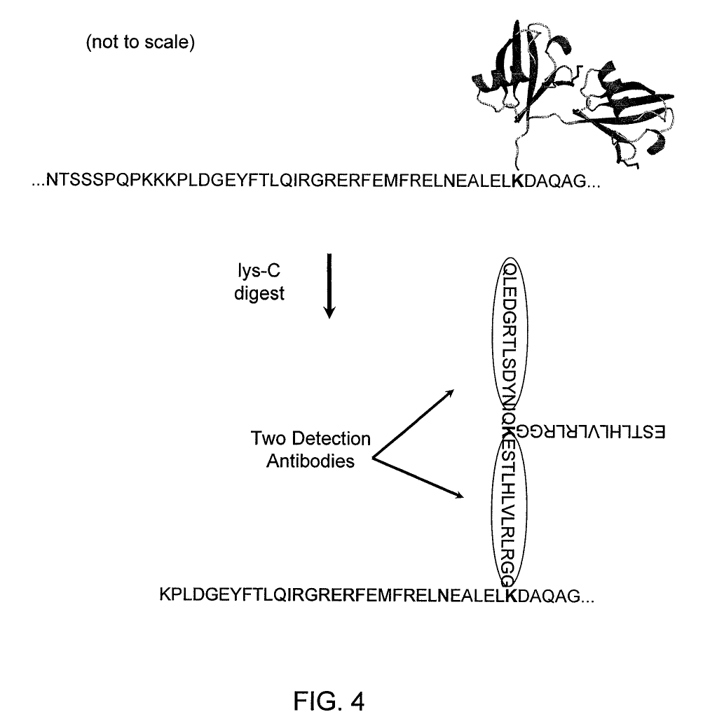

Some of these types of modifying proteins have post translational modifications of their own and form poly-protein chains (e.g., poly-ubiquitination) in the same manner as the first modifying protein is attached to the target protein. In this case, a poly-protein chain can be distinguished from a mono-protein modification in the same way. For example, as shown in FIG. 4, two binding agents can be directed against portions of a first modifying protein that, when exposed to Lys-C in the absence of a bound, second modifying protein, fragment into two peptides. If after Lys-C exposure both binding agents are present, for example, on a spot in an array, it would indicate that the Lys-C digestion of the first modifying protein was inhibited, for example, by another modifying protein covalently attached to a lysine residue on the first modifying protein. In this way, poly-versus mono-modified proteins can be differentiated.

The two methods described above and shown in FIGS. 3 and 4 respectively can be combined to yield a pattern of post translational modification at sites within one protein or within each of a plurality of proteins.

D. Other Post Translational Modifications

Other post translational modifications may also be detected/measured using the subject methods. Capture agents, such as antibodies specific for other post translationally modified residues are also readily available.

Anti-acetylated amino acid antibodies have also been commercialized. See anti-acetylated-histon H3 and H4 antibodies (Catalog #06-599 and Catalog #06-598) from Upstate Biotechnology (Lake Placid, N.Y.). In fact, Alpha Diagnostic International, Inc. (San Antonio, Tex.) offers custom synthesis of anti-acetylated amino acid antibodies.

Arginine methylation, a protein modification discovered almost 30 years ago, has recently experienced a renewed interest as several new arginine methyltransferases have been identified and numerous proteins were found to be regulated by methylation on arginine residues. Mowen and David published detailed protocols on Science's STKE website (stke dot org/cgi/content/full/OC_sigtrans;2001/93/pl1) that provide guidelines for the straightforward identification of arginine-methylated proteins, made possible by the availability of novel, commercially available reagents. Specifically, two anti-methylated arginine antibodies are described: mouse monoclonal antibody to methylarginine, clone 7E6 (IgG1) (Abeam, Cambr idge, UK) (Data sheet: abcam dot com/public/ab_detail.cfm?intAbID=412, which reacts with mono- and asymmetric dimethylated arginine residues; and mouse monoclonal antibody to methylarginine, clone 21C7 (IgM) (Abeam) (Data sheet: abeam dot com/public/ab_detail.cfm?intAbID=413), which reacts with asymmetric dimethylated arginine residues. Detailed protocols for in vitro and in vivo analysis of arginine methylation are provided. See Mowen et al., Cell 104: 731-741, 2001.

Even if there is no reported antibodies at present for certain specific modifications, it is well within the capability of a skilled artisan to raise antibodies against that specific type of modified residues. There is no compelling reason to believe that such antibodies cannot be obtained, especially in view of the prior success in raising antibodies against relatively small groups such as phosphorylated amino acids. The anti-post translational modification antibody should be checked against the same antigen that is un-modified to verify that the reactivity is depending upon the presence of the post translational modification.

As noted above, methods for detecting various types of post translational modification can be combined to yield a pattern of multiple types of modifications within one protein or within each of a plurality of proteins.

### 4. Samples and Sample Preparation

The capture agents or an array of capture agents typically are contacted with a sample, e.g., a biological fluid, a water sample, or a food sample, which has been fragmented to generate a collection of peptides, under conditions suitable for binding a PET corresponding to a protein of interest.

Samples to be assayed using the capture agents of the present invention may be drawn from various physiological, environmental or artificial sources. In particular, physiological samples such as body fluids or tissue samples of a patient or an organism may be used as assay samples. Such fluids include, but are not limited to, saliva, mucous, sweat, whole blood, serum, urine, amniotic fluid, genital fluids, fecal material, marrow, plasma, spinal fluid, pericardial fluids, gastric fluids, abdominal fluids, peritoneal fluids, pleural fluids and extraction from other body parts, and secretion from other glands. Alternatively, biological samples drawn from cells taken from the patient or grown in culture may be employed. Such samples include supernatants, whole cell lysates, or cell fractions obtained by lysis and fractionation of cellular material. Extracts of cells and fractions thereof, including those directly from a biological entity and those grown in an artificial environment, can also be used. In addition, a biological sample can be obtained and/or derived from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid.

The methods of the invention may be used for all types of samples, including insoluble proteins such as cell membrane bound and organelle membrane bound proteins (see description in US-20040180380-A1). This may be of particular interest, since many phosphoproteins are either integral membrane proteins or membrane associated proteins (e.g., by virtual of binding to an integral membrane protein, or a cytoskeleton component close to the membrane system of the cell).

In a preferred embodiment, the sample treatment is carried out under conditions to preserve the post translational modification to be detected. For example, for detection of phosphorylation, phosphohotase inhibitors may be included in the solutions used for sample preparation. Certain steps (such as the initial cell/tissue harvesting steps) may be performed at low temperatures at which phosphotases are not active.

A general method for sample preparation prior to its use in the methods of the instant invention is described herein. Briefly, a sample can be pretreated by extraction and/or dilution to minimize the interference from certain substances present in the sample. The sample can then be either chemically reduced, denatured, alkylated, or subjected to thermo-denaturation. Regardless of the denaturation step, the denatured sample is then digested by a protease, such as trypsin, before it is used in subsequent assays. A desalting step may also be added just after protease digestion if chemical denaturation if used. This process is generally simple, robust and reprodu-cible, and is generally applicable to main sample types including serum, cell lysates and tissues.

The sample may be pre treated to remove extraneous materials, stabilized, buffered, preserved, filtered, or otherwise conditioned as desired or necessary. Proteins in the sample typically are fragmented, either as part of the methods of the invention or in advance of performing these methods. Fragmentation can be performed using any art-recognized desired method, such as by using chemical cleavage (e.g., cyanogen bromide); enzymatic means (e.g., using a protease such as trypsin, chymotrypsin, pepsin, papain, carboxypeptidase, calpain, subtilisin, glut-C, endo lys-C and proteinase K, or a collection or sub-collection thereof); or physical means (e.g., fragmentation by physical shearing or fragmentation by sonication). As used herein, the terms “fragmentation” “cleavage,” “proteolytic cleavage,” “proteolysis” “restriction” and the like are used interchangeably and refer to scission of a chemical bond, typically a peptide bond, within proteins to produce a collection of peptides (i.e., protein fragments).

The purpose of the fragmentation is to generate peptides comprising PET which are soluble and available for binding with a capture agent. In essence, the sample preparation is designed to assure to the extent possible that all PET present on or within relevant proteins that may be present in the sample are available for reaction with the capture agents. This strategy can avoid many of the problems encountered with previous attempts to design protein chips caused by protein-protein complexation, post translational modifications and the like.

In one embodiment, the sample of interest is treated using a pre-determined protocol which: (A) inhibits masking of the target protein caused by target protein-protein non covalent or covalent complexation or aggregation, target protein degradation or denaturing, target protein post translational modification, or environmentally induced alteration in target protein tertiary structure, and (B) fragments the target protein to, thereby, produce at least one peptide epitope (i.e., a PET) whose concentration is directly proportional to the true concentration of the target protein in the sample. The sample treatment protocol is designed and empirically tested to result reproducibly in the generation of a PET that is available for reaction with a given capture agent. The treatment can involve protein separations; protein fractionations; solvent modifications such as polarity changes, osmolarity changes, dilutions, or pH changes; heating; freezing; precipitating; extractions; reactions with a reagent such as an endo-, exo- or site specific protease; non proteolytic digestion; oxidations; reductions; neutralization of some biological activity, and other steps known to one of skill in the art.

For example, the sample may be treated with an alkylating agent and a reducing agent in order to prevent the formation of dimers or other aggregates through disulfide/dithiol exchange. The sample of PET-containing peptides may also be treated to remove secondary modifications, including but are not limited to, phosphorylation, methylation, glycosylation, acetylation, preny-lation, using, for example, respective modification-specific enzymes such as phosphatases, etc.

In one embodiment, proteins of a sample will be denatured, reduced and/or alkylated, but will not be proteolytically cleaved. Proteins can be denatured by thermal denaturation or organic solvents, then subjected to direct detection or optionally, further proteolytic cleavage.

The use of thermal denaturation (50-90° C. for about 20 minutes) of proteins prior to enzyme digestion in solution is preferred over chemical denaturation (such as 6-8 M guanidine HCl or urea) because it does not require purification/concentration, which might be preferred or required prior to subsequent analysis. Park and Russell reported that enzymatic digestions of proteins that are resistant to proteolysis are significantly enhanced by thermal denaturation (Anal. Chem., 72 (11): 2667 -2670, 2000). Native proteins that are sensitive to proteolysis show similar or just slightly lower digestion yields following thermal denaturation. Proteins that are resistant to digestion become more susceptible to digestion, independent of protein size, following thermal denaturation. For example, amino acid sequence coverage from digest fragments increases from 15 to 86% in myoglobin and from 0 to 43% in ovalbumin. This leads to more rapid and reliable protein identification by the instant invention, especially to protease resistant proteins.

In a preferred embodiment, SDS may be used in combination with heat to facilitate optimal denaturation and (concurrent or subsequent) digestion.

Although some proteins aggregate upon thermal denaturation, the protein aggregates are easily digested by trypsin and generate sufficient numbers of digest fragments for protein identification. In fact, protein aggregation may be the reason thermal denaturation facilitates digestion in most cases. Protein aggregates are believed to be the oligomerization products of the denatured form of protein (Copeland, R. A. Methods for Protein Analysis; Chapman & Hall: New York, N.Y., 1994). In general, hydrophobic parts of the protein are located inside and relatively less hydrophobic parts of the protein are exposed to the aqueous environment. During the thermal denaturation, intact proteins are gradually unfolded into a denatured conformation and sufficient energy is provided to prevent a fold back to its native conformation. The probability for interactions with other denatured proteins is increased, thus allowing hydrophobic interactions between exposed hydrophobic parts of the proteins. In addition, protein aggregates of the denatured protein can have a more protease-labile structure than nondenatured proteins because more cleavage sites are exposed to the environment. Protein aggregates are easily digested, so that protein aggregates are not observed at the end of 3 h of trypsin digestion (Park and Russell, Anal. Chem., 72 (11): 2667 -2670, 2000). Moreover, trypsin digestion of protein aggregates generates more specific cleavage products.

Ordinary proteases such as trypsin may be used after denaturation. The process may be repeated by one or more rounds after the first round of denaturation and digestion. Alternatively, this thermal denaturation process can be further assisted by using thermophilic trypsin-like enzymes, so that denaturation and digestion can be done simultaneously. For example, Nongporn Towatana et al. (J of Bioscience and Bioengineering 87(5): 581-587, 1999) reported the purification to apparent homogeneity of an alkaline protease from culture supernatants of Bacillus sp. PS719, a novel alkaliphilic, thermophilic bacterium isolated from a thermal spring soil sample. The protease exhibited maximum activity towards azocasein at pH 9.0 and at 75° C. The enzyme was stable in the pH range 8.0 to 10.0 and up to 80° C. in the absence of Ca²⁺. This enzyme appears to be a trypsin-like serine protease, since phenylmethylsulfonyl fluoride (PMSF) and 3,4-dichloroisocoumarin (DCI) in addition to N-α-p-tosyl-L-lysine chloromethyl ketone (TLCK) completely inhibited the activity. Among the various oligopeptidyl-p-nitroanilides tested, the protease showed a preference for cleavage at arginine residues on the carboxylic side of the scissile bond of the substrate, liberating p-nitroaniline from N-carbobenzoxy (CBZ)-L-arginine-p-nitroanilide with the Kₘ and Vₘₐₓ values of 0.6 mM and 1.0 μmol min⁻¹mg protein⁻¹, respectively.

Alternatively, existing proteases may be chemically modified to achieve enhanced thermostability for use in this type of application. Mozhaev et al. (Eur J Biochem. 173(1):147-54, 1988) experimentally verified the idea presented earlier that the contact of nonpolar clusters located on the surface of protein molecules with water destabilizes proteins. It was demonstrated that protein stabilization could be achieved by artificial hydrophilization of the surface area of protein globules by chemical modification. Two experimental systems were studied for the verification of the hydrophilization approach. In one experiment, the surface tyrosine residues of trypsin were transformed to aminotyrosines using a two-step modification procedure: nitration by tetranitromethane followed by reduction with sodium dithionite. The modified enzyme was much more stable against irreversible thermo-inactivation: the stabilizing effect increased with the number of aminotyrosine residues in trypsin and the modified enzyme could become even 100 times more stable than the native one. In another experiment, alpha-chymotrypsin was covalently modified by treatment with anhydrides or chloroanhydrides of aromatic carboxylic acids. As a result, different numbers of additional carboxylic groups (up to five depending on the structure of the modifying reagent) were introduced into each Lys residue modified. Acylation of all available amino groups of alpha-chymotrypsin by cyclic anhydrides of pyromellitic and mellitic acids resulted in a substantial hydrophilization of the protein as estimated by partitioning in an aqueous Ficoll-400/Dextran-70 biphasic system. These modified enzyme preparations were extremely stable against irreversible thermal inactivation at elevated temperatures (65-98° C.); their thermostability was practically equal to the stability of proteolytic enzymes from extremely thermophilic bacteria, the most stable proteinases known to date. Similar approaches may be used to any other chosen proteases for the subject method.

In other embodiments, samples can be pre-treated with reducing agents such as β-mercaptoethanol, DTT, or TCEP (Tris(2-Carboxyethyl)Phosphine) to reduce the disulfide bonds to facilitate digestion.

Fractionation may be performed using any single or multidimentional chromatography, such as reversed phase chromatography (RPC), ion exchange chromatography, hydrophobic interaction chromatography, size exclusion chromatography, or affinity fractionation such as immunoaffinity and immobilized metal affinity chromatography. Preferably, the fractionation involves surface-mediated selection strategies. Electrophoresis, either slab gel or capillary electrophoresis, can also be used to fractionate the peptides in the sample. Examples of slab gel electrophoretic methods include sodium dodecyl sulfate polyacrylamide gel electrophoresis (SDS-PAGE) and native gel electrophoresis. Capillary electrophoresis methods that can be used for fractionation include capillary gel electrophoresis (CGE), capillary zone electrophoresis (CZE) and capillary electrochromatography (CEC), capillary isoelectric focusing, immobilized metal affinity chromatography and affinity electrophoresis.

Protein precipitation may be performed using techniques well known in the art. For example, precipitation may be achieved using known precipitants, such as potassium thiocyanate, trichloroacetic acid and ammonium sulphate.

Subsequent to fragmentation, the sample may be contacted with the capture agents of the present invention, e.g., capture agents immobilized on a planar support or on a bead, as described herein. Alternatively, the fragmented sample (containing a collection of peptides) may be fractionated based on, for example, size, post translational modifications (e.g., glycosylation or phosphorylation) or antigenic properties, and then contacted with the capture agents of the present invention, e.g., capture agents immobilized on a planar support or on a bead.

Also provided herein is an illustrative example of serum sample pre-treatment using either the thermo-denaturation or the chemical denaturation. Briefly, for thermo-denaturation, 100 μL of human serum (about 75 mg/mL total protein) is first diluted 10-fold to about 7.5 mg/mL. The diluted sample is then heated to 90° C. for 5 minutes to denature the proteins, followed by 30 minutes of trypsin digestion at 55° C. The trypsin is inactivated at 80° C. after the digestion.

For chemical denaturation, about 1.8 mL of human serum proteins diluted to about 4 mg/mL is denatured in a final concentration of 50 mM HEPES buffer (pH 8.0), 8M urea and 10 mM DTT. Iodoacetamide is then added to 25 mM final concentration. The denatured sample is then further diluted to about 1 mg/mL for protease digestion. The digested sample will pass through a desalting column before being used in subsequent assays.

Thermo-denaturation and chemical denaturation of serum proteins and cell lysates (MOLT4 and Hela cells) using the methods described herein were successful for the majority, if not all of the proteins tested, and both methods achieved comparable results in terms of protein denaturation and fragmentation.

In a preferred embodiment for certain applications, SDS is used in combination with thermal-denaturation (see, for example, Example 4). For such applications, thermal-stable proteases may be used instead of conventional proteases, especially in simultaneous denaturation and digestion.

The above examples/protocols are for illustrative purpose only, and is by no means limiting. Minor alterations of the protocol depending on specific uses can be easily achieved for optimal results in individual assays.

### 5. Selection of PET

One advantages of the PET of the instant invention is that PET can be determined in silico and generated in vitro (such as by peptide synthesis) without cloning or purifying the protein it derives from. PET is also advantageous over the full-length tryptic fragments (or for that matter, any other fragments that predictably results from any other treatments) to predictably target antibodies to defined epitopes. Though the tryptic fragment itself may be unique simply because of its length (the longer a stretch of peptide, the more likely it will be unique), antibodies raised to the tryptic fragment will target many epitopes with the fragment, some of which many not represent unique sequences. A direct implication is that, by using relatively short and unique PETs rather than the full-length (tryptic) peptide fragments, the method of the instant invention has greatly reduced, if not completely eliminated, the risk of generating antibodies that can cross react with other peptide fragments. An additional advantage may be added due to the PET selection process, such as the nearest-neighbor analysis and ranking prioritization(see below), which further eliminates the chance of cross-reactivity. All these features make the PET-based methods particularly suitable for genome-wide analysis using multiplexing techniques.

The PET of the instant invention can be selected in various ways. In the simplest embodiment, the PET for a given organism or biological sample can be generated or identified by a comprehensive search of the relevant database, using all theoretically possible PET with a given length. This process is preferably carried out computationally using, for example, any of the sequence search tools available in the art or variations thereof. For example, to identify PET of 5 amino acids in length (a total of 3.2 million possible PET candidates, see table 2.2.2 below), each of the 3.2 million candidates may be used as a query sequence to search against the human proteome as described below. Any candidate that has more than one hit (found in two or more proteins) is immediately eliminated before further searching is done. At the end of the search, a list of human proteins that have one or more PETs can be obtained. The same or similar procedure can be used for any pre-determined organism or database.

For example, PETs for each human protein can be identified using the following procedure. A Perl program is developed to calculate the occurrence of all possible peptides, given by 20^{N}, of defined length N (amino acids) in human proteins. For example, the total tag space is 160,000 (20⁴) for tetramer peptides, 3.2 M (20⁵) for pentamer peptides, and 64 M (20⁶) for hexamer peptides, so on. Predicted human protein sequences are analyzed for the presence or absence of all possible peptides of N amino acids. PET are the peptide sequences that occur only once in the human proteome. Thus the presence of a specific PET is an intrinsic property of the protein sequence and is operational independent. According to this approach, a definitive set of PETs can be defined and used regardless of the sample processing procedure (operational independence).

In one embodiment, to speed up the searching process, computer algorithms may be developed or modified to eliminate unnecessary searches before the actual search begins.

Using the example above, two highly related (say differ only in a few amino acid positions) human proteins may be aligned, and a large number of candidate PET can be eliminated based on the sequence of the identical regions. For example, if there is a stretch of identical sequence of 20 amino acids, then sixteen 5-amino acid PETs can be eliminated without searching, by virtue of their simultaneous appearance in two non-identical human proteins. This elimination process can be continued using as many highly related protein pairs or families as possible, such as the evolutionary conserved proteins such as histones, globins, etc.

In another embodiment, the identified PET for a given protein may be rank-ordered based on certain criteria, so that higher ranking PETs are preferred to be used in generating specific capture agents.

For example, certain PET may naturally exist on protein surface, thus making good candidates for being a soluble peptide when digested by a protease. On the other hand, certain PET may exist in an internal or core region of a protein, and may not be readily soluble even after digestion. Such solubility property may be evaluated by available software. The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used. Surface PETs generally have higher ranking than internal PETs. In one embodiment, the log P or log D values that can be calculated for a PET, or proteolytic fragment containing a PET, can be calculated and used to rank order the PET's based on likely solubility under conditions that a protein sample is to be contacted with a capture agent.

Regardless of the manner the PETs are generated, for many applications, an ideal PET preferably is 8 amino acids in length, and the parental tryptic peptide should be smaller than 20 amino acid long. However, for the subject sandwich immunoassays, the parental fragment must be long enough to support simultaneous binding by two antibodies. Since antibodies typically recognize peptide epitopes of 4-8 amino acids, the preferred length of polypeptide fragments used for the subject sandwich immunoassays is generally at least about 15 amino acids long, 20 amino acids long, 25 amino acids long, or about 30 amino acids long. These peptides of about 12-20 amino acids are also conventionally used for antibody production.

In certain embodiments, a protease that tends to generate (on average) the target length of polypeptide fragments is preferred. For example, LysC is a preferred enzyme (over trypsin) for most sandwich immunoassay applications, since the average fragment size for LysC is slightly longer than that of trypsin.

Since trypsin is a preferred digestion enzyme in certain embodiments, a PET in these embodiments should not contain K or R in the middle of the sequence so that the PET will not be cleaved by trypsin during sample preparation. In a more general sense, the selected PET should not contain or overlap a digestion site such that the PET is expected to be destroyed after digestion, unless an assay specifically prefer that a PET be destroyed after digestion.

In addition, an ideal PET preferably does not have hydrophobic parental tryptic peptide, is highly antigenic, and has the smallest numbers (preferably none) of closest related peptides (nearest neighbor peptides or NNP) defined by nearest neighbor analysis.

Any PET may also be associated with an annotation, which may contain useful information such as: whether the PET may be destroyed by a certain protease (such as trypsin), whether it is likely to appear on a digested peptide with a relatively rigid or flexible structure, etc. These characteristics may help to rank order the PETs for use if generating specific capture agents, especially when there are a large number of PETs associated with a given protein. Since PET may change depending on particular use in a given organism, ranking order may change depending on specific usages. A PET may be low ranking due to its probability of being destroyed by a certain protease may rank higher in a different fragmentation scheme using a different protease.

In another embodiment, the computational algorithm for selecting optimal PET from a protein for antibody generation takes antibody-peptide interaction data into consideration. A process such as Nearest-Neighbor Analysis (NNA), can be used to select most unique PET for each protein. Each PET in a protein is given a relative score, or PET Uniqueness Index, that is based on the number of nearest neighbors it has. The higher the PET Uniqueness Index, the more unique the PET is. The PET Uniqueness Index can be calculated using an Amino Acid Replacement Matrix such as the one in Table VIII of Getzoff, E D, Tainer J A and Lerner R A.

The chemistry and mechanism of antibody binding to protein antigens. 1988. Advances. Immunol. 43: 1-97. In this matrix, the replaceability of each amino acid by the remaining 19 amino acids was calculated based on experimental data on antibody cross-reactivity to a large number of peptides of single mutations (replacing each amino acid in a peptide sequence by the remaining 19 amino acids). For example, each octamer PET from a protein is compared to 8.7 million octamers present in human proteome and a PET Uniqueness Index is calculated. This process not only selects the most unique PET for particular protein, it also identifies Nearest Neighbor Peptides for this PET. This becomes important for defining cross-reactivity of PET-specific antibodies since Nearest Neighbor Peptides are the ones most likely will cross-react with particular antibody.

Besides PET Uniqueness Index, the following parameters for each PET may also be calculated and help to rank the PETs:
(a) PET Solubility Index: which involves calculating Log P and Log D of the PET.
(b) PET Hydrophobicity and water accessibility: only hydrophilic peptides and peptides with good water accessibility will be selected.
(c) PET Length: since longer peptides tend to have conformations in solution, we use PET peptides with defined length of 8 amino acids. PET-specific antibodies will have better defined specificity due to limited number of epitopes in these shorter peptide sequences. This is very important for multiplexing assays using these antibodies. In one embodiment, only antibodies generated by this way will be used for multiplexing assays.
(d) Evolutionary Conservation Index: each human PET will be compared with other species to see whether a PET sequence is conserved cross species. Ideally, PET with minimal conservation, for example, between mouse and human sequences will be selected. This will maximize the possibility to generate good immunoresponse and monoclonal antibodies in mouse.

### 6. Capture Agents

According to the instant invention, the (first and second) capture agents used should be capable of selective affinity reactions with PET moieties. Generally, such interaction will be non-covalent in nature, though the present invention also contemplates the use of capture reagents that become covalently linked to the PET.

Examples of capture agents which can be used include, but are not limited to: nucleotides; nucleic acids including oligonucleotides, double stranded or single stranded nucleic acids (linear or circular), nucleic acid aptamers and ribozymes; PNA (peptide nucleic acids); proteins, including antibodies (such as monoclonal or recombinantly engineered antibodies or antibody fragments), T cell receptor and MHC complexes, lectins and scaffolded peptides; peptides; other naturally occurring polymers such as carbohydrates; artificial polymers, including plastibodies; small organic molecules such as drugs, metabolites and natural products; and the like. Preferred capture agents are antibodies generated in animals against synthetic peptides. Both monoclonal and polyclonal preparations can be used.

In certain embodiments, the capture agents are immobilized, permanently or reversibly, on a solid support such as a bead, chip, or slide. When employed to analyze a complex mixture of proteins, the immobilized capture agent are arrayed and/or otherwise labeled for deconvolution of the binding data to yield identity of the capture agent (and therefore of the protein to which it binds) and (optionally) to quantitate binding. Alternatively, the capture agents can be provided free in solution (soluble), and other methods can be used for deconvolving PET binding in parallel.

In one embodiment, the capture agents are conjugated with a reporter molecule such as a fluorescent molecule or an enzyme, and used to detect the presence of bound PET on a substrate (such as a chip or bead), in for example, a “sandwich” type assay in which one capture agent is immobilized on a support to capture a PET, while a second, labeled capture agent also specific for the captured PET may be added to detect/quantitate the captured PET. In this embodiment, the peptide fragment contains two unique, non-overlapping PETs, one recognized by the immobilized the capture agent, the other recognized by the lablled detecting capture agent. In a related embodiment, one PET unique to the peptide fragment can be used in conjunction with a common PET shared among several protein family members or splicing isoforms. The spatial arrangement of these two PET is such that binding by one capture agent will not substantially affect the binding by the other capture agent (for example, the binding sites may be separated by a few amino acids). In addition, the length of the peptide fragment is such that it encompasses two PETs properly spaced from each other. Preferably, peptide fragments are at least about 15 residues for sandwich assay. In other embodiments a labeled-PET peptide is used in a competitive binding assay to determine the amount of unlabeled PET (from the sample) that binds to the capture agent. In this embodiment, the peptide fragment need only be long enough to encompass one PET, so peptides as short as 5-8 residues may be suitable.

Generally, the sandwich assay tend to be more (e.g., about 10, 100, or 1000 fold more) sensitive than the competitive binding assay.

An important advantage of the invention is that useful capture agents can be identified and/or synthesized even in the absence of a sample of the protein to be detected. With the completion of the whole genome in a number of organisms, such as human, fly (e.g., Drosophila melanogaster) and nematode (e.g., C. elegans), PET of a given length or combination thereof can be identified for any single given protein in a certain organism, and capture agents for any of these proteins of interest can then be made without ever cloning and expressing the full length protein.

In addition, the suitability of any PET to serve as an antigen or target of a capture agent can be further checked against other available information. For example, since amino acid sequence of many proteins can now be inferred from available genomic data, sequence from the structure of the proteins unique to the sample can be determined by computer aided searching, and the location of the peptide in the protein, and whether it will be accessible in the intact protein, can be determined. Once a suitable PET peptide is found, it can be synthesized using known techniques. With a sample of the PET in hand, an agent that interacts with the peptide such as an antibody or peptidic binder, can be raised against it or panned from a library. In this situation, care must be taken to assure that any chosen fragmentation protocol for the sample does not restrict the protein in a way that destroys or masks the PET. This can be determined theoretically and/or experimentally, and the process can be repeated until the selected PET is reliably retrieved by a capture agent(s).

The PET set selected according to the teachings of the present invention can be used to generate peptides either through enzymatic cleavage of the protein from which they were generated and selection of peptides, or preferably through peptide synthesis methods.

Proteolytically cleaved peptides can be separated by chromatographic or electrophoretic procedures and purified and renatured via well known prior art methods.

Synthetic peptides can be prepared by classical methods known in the art, for example, by using standard solid phase techniques. The standard methods include exclusive solid phase synthesis, partial solid phase synthesis methods, fragment condensation, classical solution synthesis, and even by recombinant DNA technology. See, e.g., Merrifield, J. Am. Chem. Soc., 85:2149 (1963). Solid phase peptide synthesis procedures are well known in the art and further described by John Morrow Stewart and Janis Dillaha Young, Solid Phase Peptide Syntheses (2nd Ed., Pierce Chemical Company, 1984).

Synthetic peptides can be purified by preparative high performance liquid chromatography [Creighton T. (1983) Proteins, structures and molecular principles. WH Freeman and Co. N.Y.] and the composition of which can be confirmed via amino acid sequencing.

In addition, other additives such as stabilizers, buffers, blockers and the like may also be provided with the capture agent.

A. Antibodies

In one preferred embodiment, the capture agent is an antibody or an antibody-like molecule (collectively “antibody”). Thus an antibody useful as capture agent may be a full length antibody or a fragment thereof, which includes an “antigen-binding portion” of an antibody. The term “antigen-binding portion,” as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P., et al. (1993) Proc. Natl. Acad. Sci. USA 90:6444-6448; Poljak, R. J., et al. (1994) Structure 2:1121-1123).

Still further, an antibody or antigen-binding portion thereof may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

Antibodies may be polyclonal or monoclonal. The terms “monoclonal antibodies” and “monoclonal antibody composition,” as used herein, refer to a population of antibody molecules that contain only one species of an antigen binding site capable of immunoreacting with a particular epitope of an antigen, whereas the term “polyclonal antibodies” and “polyclonal antibody composition” refer to a population of antibody molecules that contain multiple species of antigen binding sites capable of interacting with a particular antigen. A monoclonal antibody composition, typically displays a single binding affinity for a particular antigen with which it immunoreacts.

Any art-recognized methods can be used to generate a PET-directed antibody. For example, a PET (alone or linked to a hapten) can be used to immunize a suitable subject, (e.g., rabbit, goat, mouse or other mammal or vertebrate). For example, the methods described in U.S. Pat. Nos. 5,422,110; 5,837,268; 5,708,155; 5,723,129; and 5,849,531 can be used. The immunogenic preparation can further include an adjuvant, such as Freund's complete or incomplete adjuvant, or similar immunostimulatory agent. Immunization of a suitable subject with a PET induces a polyclonal anti-PET antibody response. The anti-PET antibody titer in the immunized subject can be monitored over time by standard techniques, such as with an enzyme linked immunosorbent assay (ELISA) using immobilized PET.

The antibody molecules directed against a PET can be isolated from the mammal (e.g., from the blood) and further purified by well known techniques, such as protein A chromatography to obtain the IgG fraction. At an appropriate time after immunization, e.g., when the anti-PET antibody titers are highest, antibody-producing cells can be obtained from the subject and used to prepare, e.g., monoclonal antibodies by standard techniques, such as the hybridoma technique originally described by Kohler and Milstein (1975) Nature 256:495-497) (see also, Brown et al. (1981) J. Immunol. 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol Today 4:72), or the EBV-hybridoma technique (Cole et al. (1985), Monoclonal Antibodies and Cancer Therapy, Alan R. Liss, Inc., pp. 77-96). The technology for producing monoclonal antibody hybridomas is well known (see generally R. H. Kenneth, in Monoclonal Antibodies: A New Dimension In Biological Analyses, Plenum Publishing Corp., New York, N.Y. (1980); E. A. Lerner (1981) Yale J. Biol. Med., 54:387-402; M. L. Gefter et al. (1977) Somatic Cell Genet. 3:231-36). Briefly, an immortal cell line (typically a myeloma) is fused to lymphocytes (typically splenocytes) from a mammal immunized with a PET immunogen as described above, and the culture supernatants of the resulting hybridoma cells are screened to identify a hybridoma producing a monoclonal antibody that binds a PET.

Any of the many well known protocols used for fusing lymphocytes and immortalized cell lines can be applied for the purpose of generating an anti-PET monoclonal antibody (see, e.g., G. Galfre et al. (1977) Nature 266:55052; Gefter et al. Somatic Cell Genet., cited supra; Lerner, Yale J. Biol. Med., cited supra; Kenneth, Monoclonal Antibodies, cited supra). Moreover, the ordinarily skilled worker will appreciate that there are many variations of such methods which also would be useful. Typically, the immortal cell line (e.g., a myeloma cell line) is derived from the same mammalian species as the lymphocytes. For example, murine hybridomas can be made by fusing lymphocytes from a mouse immunized with an immunogenic preparation of the present invention with an immortalized mouse cell line. Preferred immortal cell lines are mouse myeloma cell lines that are sensitive to culture medium containing hypoxanthine, aminopterin and thymidine (“HAT medium”). Any of a number of myeloma cell lines can be used as a fusion partner according to standard techniques, e.g., the P3-NS1/1-Ag4-1, P3-x63-Ag8.653 or Sp2/O-Ag14 myeloma lines. These myeloma lines are available from ATCC. Typically, HAT-sensitive mouse myeloma cells are fused to mouse splenocytes using polyethylene glycol (“PEG”). Hybridoma cells resulting from the fusion are then selected using HAT medium, which kills unfused and unproductively fused myeloma cells (unfused splenocytes die after several days because they are not transformed). Hybridoma cells producing a monoclonal antibody of the invention are detected by screening the hybridoma culture supernatants for antibodies that bind a PET, e.g., using a standard ELISA assay.

In addition, automated screening of antibody or scaffold libraries against arrays of target proteins/PETs will be the most rapid way of developing thousands of reagents that can be used for protein expression profiling. Furthermore, polyclonal antisera, hybridomas or selection from library systems may also be used to quickly generate the necessary capture agents. A high-throughput process for antibody isolation is described by Hayhurst and Georgiou in Curr Opin Chem Biol 5(6):683-9, December 2001.

The PET antigens used for the generation of PET-specific antibodies are preferably blocked at either the N- or C-terminal end, most preferably at both ends to generate neutral groups, since antibodies raised against peptides with non-neutralized ends may not be functional for the methods of the invention. The PET antigens can be most easily synthesized using standard molecular biology or chemical methods, for example, with a peptide synthesizer. The terminals can be blocked with NH2- or COO— groups as appropriate, or any other blocking agents to eliminate free ends. In a preferred embodiment, one end (either N- or C-terminus) of the PET will be conjugated with a carrier protein such as KLH or BSA to facilitate antibody generation. KLH represents Keyhole-limpet hemocyanin, an oxygen carrying copper protein found in the keyhole-limpet (Megathura crenulata), a primitive mollusk sea snail. KLH has a complex molecular arrangement and contains a diverse antigenic structure and elicits a strong nonspecific immune response in host animals. Therefore, when small peptides (which may not be very immunogenic) are used as immunogens, they are preferably conjugated to KLH or other carrier proteins (BSA) for enhanced immune responses in the host animal. The resulting antibodies can be affinity purified using a polypeptide corresponding to the PET-containing tryptic peptide of interest.

Blocking the ends of PET in antibody generation may be advantageous, since in many (if not most) cases, the selected PETs are contained within larger (tryptic) fragments. In these cases, the PET-specific antibodies are required to bind PETs in the middle of a peptide fragment. Therefore, blocking both the C- and N-terminus of the PETs best simulates the antibody binding of peptide fragments in a digested sample. Similarly, if the selected PET sequence happens to be at the N- or C-terminal end of a target fragment, then only the other end of the immunogen needs to be blocked, preferably by a carrier such as KLH or BSA.

In a preferred embodiment, an improved method may be used to generate antibodies against target protein, e.g., small peptide fragments, such as synthesized peptides.

Specifically, a new approach is used to design immunogens and purify antibodies, in order to generate a highly specific polyclonal pool, targeting precisely the PET sequence in the context of how it is presented in the peptide fragment produced by digestion of the sample.

While not wishing to be bound by any particular theory, it is believed that: when one immunizes animal with a short peptide, one end of the peptide must be covalently attached to a carrier protein. But since the other end has no structure to it, it moves like “flopping in the wind.” It is common for antibodies that are generated as a response to this immunogen to target the “free” end of the peptide, as the energies favor that end to fit into the binding pocket necessary for antibody induction. Applicants found that antibodies thus generated may bind poorly to the exact same peptide sequence, if the end is no longer free. This is an important consideration if one is targeting a peptide sequence that lies within a longer peptide fragment, as is typical, as the PET is formed by a small segment of the peptide. Further, when making polyclonal antibodies, even if some of the “clones” have desirable binding to the longer peptide fragment, if the majority of “clones” target the free end, then the antibody pool will have limited utility.

To solve this problem, the immunogen is prepared such that the target PET is put in a construct where a physical structure constrains both ends (Applicants have used a GSG linker, but there are many others that can be used). That way, antibodies that target the entire PET sequence “see” the sequence in the context of the physical rigidity on the free end that it will encounter in the digested sample. For harvesting polyclonal antibodies, the next step is to purify the antibody using the PET sequence itself, but substituting the linker that was used on the immunogen with a different linker (so as not to purify antibodies to the linker used in the immunogen). The recommended approach is to use the native protein sequence that surrounds the selected PET as the linker. Part of the reason to use a different linker for purification may be that antibodies that bind to the linker region are not selected. For production of monoclonal antibodies, the strategy is to screen the clones against the purification peptide described above, for similar reasons.

When generating PET-specific antibodies, preferably monoclonal antibodies, a peptide immunogen comprising essentially of the target PET sequence may be administered to an animal according to standard antibody generation protocol for short peptide antigens. In one embodiment, the short peptide antigen may be conjugated with a carrier such as KLH. However, when screening for antibodies specific for the PET sequence, it is preferred that the parental peptide fragments containing the PET sequence (such as the fragment resulting from trypsin digestion) is used. This ensures that the identified antibodies will be not only specific for the original PET sequence, but also able to recognize the PET peptide fragment for which the antibody is designed. Optionally, the specificity of the identified antibody can be further verified by reacting with the original immunogen such as the end-blocked PET sequence itself.

In certain embodiments, several different immunogens for different PET sequences may be simultaneously administered to the same animal, so that different antibodies may be generated in one animal. Obviously, for each immunogen, a separate screen would be needed to identify antibodies specific for the immunogen.

In an alternative embodiment, different PETs may be linked together in a single, longer immunogen for administration to an animal. The linker sequence can be flexible linkers such as GS, GSSSS or repeats thereof (such as three-peats).

In both embodiments described above, the different immunogens may be from the same or different organisms or proteomes. These methods are all potential means of reducing costs in antibody generation. An unexpected advantage of using linked PET sequences as immunogen is that longer immunogens may at certain situations produce higher affinity antibodies than those produced using short PET sequences.

B. Proteins and Peptides

Other methods for generating the capture agents of the present invention include phage-display technology described in, for example, Dower et al., WO 91/17271, McCafferty et al., WO 92/01047, Herzig et al., U.S. Pat. No. 5,877,218, Winter et al., U.S. Pat. No. 5,871,907, Winter et al., U.S. Pat. No. 5,858,657, Holliger et al., U.S. Pat. No. 5,837,242, Johnson et al., U.S. Pat. No. 5,733,743 and Hoogenboom et al., U.S. Pat. No. 5,565,332. In these methods, libraries of phage are produced in which members display different antibodies, antibody binding sites, or peptides on their outer surfaces. Antibodies are usually displayed as Fv or Fab fragments. Phage displaying sequences with a desired specificity are selected by affinity enrichment to a specific PET.

Methods such as yeast display and in vitro ribosome display may also be used to generate the capture agents of the present invention. The foregoing methods are described in, for example, Methods in Enzymology Vol 328—Part C: Protein-protein interactions & Genomics and Bradbury A. (2001) Nature Biotechnology 19:528-529.

In a related embodiment, proteins or polypeptides may also act as capture agents of the present invention. These peptide capture agents also specifically bind to an given PET, and can be identified, for example, using phage display screening against an immobilized PET, or using any other art-recognized methods. Once identified, the peptidic capture agents may be prepared by any of the well known methods for preparing peptidic sequences. For example, the peptidic capture agents may be produced in prokaryotic or eukaryotic host cells by expression of polynucleotides encoding the particular peptide sequence. Alternatively, such peptidic capture agents may be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken, I. M. (1981) CRC Crit. Rev. Biochem. 11:255; Kaiser et al. (1989) Science 243:187; Merrifield, B. (1986) Science 232:342; Kent, S. B. H. (1988) Ann. Rev. Biochem. 57:957; and Offord, R. E. (1980) Semisynthetic Proteins, Wiley Publishing.

The peptidic capture agents may also be prepared by any suitable method for chemical peptide synthesis, including solution-phase and solid-phase chemical synthesis. Preferably, the peptides are synthesized on a solid support. Methods for chemically synthesizing peptides are well known in the art (see, e.g., Bodansky, M. Principles of Peptide Synthesis, Springer Verlag, Berlin (1993) and Grant, G. A (ed.). Synthetic Peptides: A User's Guide, W.H. Freeman and Company, New York (1992). Automated peptide synthesizers useful to make the peptidic capture agents are commercially available.

C. Scaffolded Peptides

An alternative approach to generating capture agents for use in the present invention makes use of antibodies are scaffolded peptides, e.g., peptides displayed on the surface of a protein. The idea is that restricting the degrees of freedom of a peptide by incorporating it into a surface-exposed protein loop could reduce the entropic cost of binding to a target protein, resulting in higher affinity. Thioredoxin, fibronectin, avian pancreatic polypeptide (aPP) and albumin, as examples, are small, stable proteins with surface loops that will tolerate a great deal of sequence variation. To identify scaffolded peptides that selectively bind a target PET, libraries of chimeric proteins can be generated in which random peptides are used to replace the native loop sequence, and through a process of affinity maturation, those which selectively bind a PET of interest are identified.

D. Simple Peptides and Peptidomimetic Compounds

Peptides are also attractive candidates for capture agents because they combine advantages of small molecules and proteins. Large, diverse libraries can be made either biologically or synthetically, and the “hits” obtained in binding screens against PET moieties can be made synthetically in large quantities.

Peptide-like oligomers (Soth et al. (1997) Curr. Opin. Chem. Biol. 1:120-129) such as peptoids (Figliozzi et al., (1996) Methods Enzymol. 267:437-447) can also be used as capture reagents, and can have certain advantages over peptides. They are impervious to proteases and their synthesis can be simpler and cheaper than that of peptides, particularly if one considers the use of functionality that is not found in the 20 common amino acids.

E. Nucleic Acids

In another embodiment, aptamers binding specifically to a PET may also be used as capture agents. As used herein, the term “aptamer,” e.g., RNA aptamer or DNA aptamer, includes single-stranded oligonucleotides that bind specifically to a target molecule. Aptamers are selected, for example, by employing an in vitro evolution protocol called systematic evolution of ligands by exponential enrichment. Aptamers bind tightly and specifically to target molecules; most aptamers to proteins bind with a K_{d} (equilibrium dissociation constant) in the range of 1 pM to 1 nM. Aptamers and methods of preparing them are described in, for example, E. N. Brody et al. (1999) Mol. Diagn. 4:381-388.

In one embodiment, the subject aptamers can be generated using SELEX, a method for generating very high affinity receptors that are composed of nucleic acids instead of proteins. See, for example,. Brody et al. (1999) Mol. Diagn. 4:381-388. SELEX offers a completely in vitro combinatorial chemistry alternative to traditional protein-based antibody technology. Similar to phage display, SELEX is advantageous in terms of obviating animal hosts, reducing production time and labor, and simplifying purification involved in generating specific binding agents to a particular target PET.

To further illustrate, SELEX can be performed by synthesizing a random oligonucleotide library, e.g., of greater than 20 bases in length, which is flanked by known primer sequences. Synthesis of the random region can be achieved by mixing all four nucleotides at each position in the sequence. Thus, the diversity of the random sequence is maximally 4ⁿ, where n is the length of the sequence, minus the frequency of palindromes and symmetric sequences. The greater degree of diversity conferred by SELEX affords greater opportunity to select for oligonuclotides that form 3-dimensional binding sites. Selection of high affinity oligonucleotides is achieved by exposing a random SELEX library to an immobilized target PET. Sequences, which bind readily without washing away, are retained and amplified by the PCR, for subsequent rounds of SELEX consisting of alternating affinity selection and PCR amplification of bound nucleic acid sequences. Four to five rounds of SELEX are typically sufficient to produce a high affinity set of aptamers.

Therefore, hundreds to thousands of aptamers can be made in an economically feasible fashion. Blood and urine can be analyzed on aptamer chips that capture and quantitate proteins. SELEX has also been adapted to the use of 5-bromo (5-Br) and 5-iodo (5-I) deoxyuridine residues. These halogenated bases can be specifically cross-linked to proteins. Selection pressure during in vitro evolution can be applied for both binding specificity and specific photo-cross-linkability. These are sufficiently independent parameters to allow one reagent, a photo-cross-linkable aptamer, to substitute for two reagents, the capture antibody and the detection antibody, in a typical sandwich array. After a cycle of binding, washing, cross-linking, and detergent washing, proteins will be specifically and covalently linked to their cognate aptamers. Because no other proteins are present on the chips, protein-specific stain will now show a meaningful array of pixels on the chip. Combined with learning algorithms and retrospective studies, this technique should lead to a robust yet simple diagnostic chip.

In yet another related embodiment, a capture agent may be an allosteric ribozyme. The term “allosteric ribozymes,” as used herein, includes single-stranded oligonucleotides that perform catalysis when triggered with a variety of effectors, e.g., nucleotides, second messengers, enzyme cofactors, pharmaceutical agents, proteins, and oligonucleotides. Allosteric ribozymes and methods for preparing them are described in, for example, S. Seetharaman et al. (2001) Nature Biotechnol. 19: 336-341. According to Seetharaman et al., a prototype biosensor array has been assembled from engineered RNA molecular switches that undergo ribozyme-mediated self-cleavage when triggered by specific effectors. Each type of switch is prepared with a 5′-thiotriphosphate moiety that permits immobilization on gold to form individually addressable pixels. The ribozymes comprising each pixel become active only when presented with their corresponding effector, such that each type of switch serves as a specific analyte sensor. An addressed array created with seven different RNA switches was used to report the status of targets in complex mixtures containing metal ion, enzyme cofactor, metabolite, and drug analytes. The RNA switch array also was used to determine the phenotypes of Escherichia coli strains for adenylate cyclase function by detecting naturally produced 3′,5′-cyclic adenosine monophosphate (cAMP) in bacterial culture media.

F. Plastibodies

In certain embodiments the subject capture agent is a plastibody. The term “plastibody” refers to polymers imprinted with selected template molecules. See, for example, Bruggemann (2002) Adv Biochem Eng Biotechnol 76:127-63; and Haupt et al. (1998) Trends Biotech. 16:468-475. The plastibody principle is based on molecular imprinting, namely, a recognition site that can be generated by stereoregular display of pendant functional groups that are grafted to the sidechains of a polymeric chain to thereby mimic the binding site of, for example, an antibody.

G. Chimeric Binding Agents Derived from Two Low-Affinity Ligands

Still another strategy for generating suitable capture agents is to link two or more modest-affinity ligands and generate high affinity capture agent. Given the appropriate linker, such chimeric compounds can exhibit affinities that approach the product of the affinities for the two individual ligands for the PET. To illustrate, a collection of compounds is screened at high concentrations for weak interactors of a target PET. The compounds that do not compete with one another are then identified and a library of chimeric compounds is made with linkers of different length. This library is then screened for binding to the PET at much lower concentrations to identify high affinity binders. Such a technique may also be applied to peptides or any other type of modest-affinity PET-binding compound.

H. Labels for Capture Agents

The capture agents of the present invention may be modified to enable detection using techniques known to one of ordinary skill in the art, such as fluorescent, radioactive, chromatic, optical, and other physical or chemical labels, as described herein below.

For example, the capture agents or reagents used for detection may be labeled by an enzyme, a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle, etc.

I. Miscellaneous

In addition, for any given PET, multiple capture agents belonging to each of the above described categories of capture agents may be available. These multiple capture agents may have different properties, such as affinity/avidity/specificity for the PET. Different affinities are useful in covering the wide dynamic ranges of expression which some proteins can exhibit. Depending on specific use, in any given array of capture agents, different types/amounts of capture agents may be present on a single chip/array to achieve optimal overall performance.

In a preferred embodiment, capture agents are raised against PETs that are located on the surface of the protein of interest, e.g., hydrophilic regions. PETs that are located on the surface of the protein of interest may be identified using any of the well known software available in the art. For example, the Naccess program may be used.

Naccess is a program that calculates the accessible area of a molecule from a PDB (Protein Data Bank) format file. It can calculate the atomic and residue accessibilities for both proteins and nucleic acids. Naccess calculates the atomic accessible area when a probe is rolled around the Van der Waal's surface of a macromolecule. Such three-dimensional co-ordinate sets are available from the PDB at the Brookhaven National laboratory. The program uses the Lee & Richards (1971) J. Mol. Biol., 55, 379-400 method, whereby a probe of given radius is rolled around the surface of the molecule, and the path traced out by its center is the accessible surface.

The solvent accessibility method described in Boger, J., Emini, E. A. & Schmidt, A., Surface probability profile-An heuristic approach to the selection of synthetic peptide antigens, Reports on the Sixth International Congress in Immunology (Toronto) 1986 p. 250 also may be used to identify PETs that are located on the surface of the protein of interest. The package MOLMOL (Koradi, R. et al. (1996) J. Mol. Graph. 14:51-55) and Eisenhaber's ASC method (Eisenhaber and Argos (1993) J. Comput. Chem. 14:1272-1280; Eisenhaber et al. (1995) J. Comput. Chem. 16:273-284) may also be used.

In another embodiment, capture agents are raised that are designed to bind with peptides generated by digestion of intact proteins rather than with accessible peptidic surface regions on the proteins. In this embodiment, it is preferred to employ a fragmentation protocol which reproducibly generates all of the PETs in the sample under study.

### 7. Arrays

In certain embodiments, the capture agents need to be immobilized onto a solid support (e.g., a planar support or a bead) to construct arrays, e.g., high-density arrays, of capture agents for efficient screening of complex chemical or biological samples or large numbers of compounds. A variety of methods are known in the art for attaching biological molecules to solid supports. See, generally, Affinity Techniques, Enzyme Purification: Part B, Meth. Enz. 34 (ed. W. B. Jakoby and M. Wilchek, Acad. Press, N.Y. 1974) and Immobilized Biochemicals and Affinity Chromatography, Adv. Exp. Med. Biol. 42 (ed. R. Dunlap, Plenum Press, N.Y. 1974). The following are a few considerations when constructing arrays.

A. Formats and Surfaces Consideration

Protein arrays have been designed as a miniaturisation of familiar immunoassay methods such as ELISA and dot blotting, often utilizing fluorescent readout, and facilitated by robotics and high throughput detection systems to enable multiple assays to be carried out in parallel. Common physical supports include glass slides, silicon, microwells, nitrocellulose or PVDF membranes, and magnetic and other microbeads. While microdrops of protein delivered onto planar surfaces are widely used, related alternative architectures include CD centrifugation devices based on developments in microfluidics [Gyros] and specialized chip designs, such as engineered microchannels in a plate [The Living Chip™, Biotrove] and tiny 3D posts on a silicon surface [Zyomyx]. Particles in suspension can also be used as the basis of arrays, providing they are coded for identification; systems include color coding for microbeads [Luminex, Bio-Rad] and semiconductor nanocrystals [QDots™, Quantum Dots], and barcoding for beads [UltraPlex™, Smartbeads] and multimetal microrods [Nanobarcodes™ particles, Surromed]. Beads can also be assembled into planar arrays on semiconductor chips [LEAPS technology, BioArray Solutions].

B. Immobilization Considerations

The variables in immobilization of proteins such as antibodies include both the coupling reagent and the nature of the surface being coupled to. Ideally, the immobilization method used should be reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally requires site-specific labeling of the protein.

The properties of a good protein array support surface are that it should be chemically stable before and after the coupling procedures, allow good spot morphology, display minimal nonspecific binding, not contribute a background in detection systems, and be compatible with different detection systems.

Both covalent and noncovalent methods of protein immobilization are used and have various pros and cons. Passive adsorption to surfaces is methodologically simple, but allows little quantitative or orientational control; it may or may not alter the functional properties of the protein, and reproducibility and efficiency are variable. Covalent coupling methods provide a stable linkage, can be applied to a range of proteins and have good reproducibility; however, orientation may be variable, chemical dramatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents [Telechem]. In the Versalinx™ system [Prolinx], reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ [PerkinElmer], based on a 3-dimensional polyacrylamide gel; this substrate is reported to give a particularly low background on glass microarrays, with a high capacity and retention of protein function. Widely used biological capture methods are through biotin/streptavidin or hexahistidine/Ni interactions, having modified the protein appropriately. Biotin may be conjugated to a poly-lysine backbone immobilized on a surface such as titanium dioxide [Zyomyx] or tantalum pentoxide [Zeptosens].

Arenkov et al., for example, have described a way to immobilize proteins while preserving their function by using microfabricated polyacrylamide gel pads to capture proteins, and then accelerating diffusion through the matrix by microelectrophoresis (Arenkov et al. (2000), Anal Biochem 278(2):123-31). The patent literature also describes a number of different methods for attaching biological molecules to solid supports. For example, U.S. Pat. No. 4,282,287 describes a method for modifying a polymer surface through the successive application of multiple layers of biotin, avidin, and extenders. U.S. Pat. No. 4,562,157 describes a technique for attaching biochemical ligands to surfaces by attachment to a photochemically reactive arylazide. U.S. Pat. No. 4,681,870 describes a method for introducing free amino or carboxyl groups onto a silica matrix, in which the groups may subsequently be covalently linked to a protein in the presence of a carbodiimide. In addition, U.S. Pat. No. 4,762,881 describes a method for attaching a polypeptide chain to a solid substrate by incorporating a light-sensitive unnatural amino acid group into the polypeptide chain and exposing the product to low-energy ultraviolet light.

The surface of the support is chosen to possess, or is chemically derivatized to possess, at least one reactive chemical group that can be used for further attachment chemistry. There may be optional flexible adapter molecules interposed between the support and the capture agents. In one embodiment, the capture agents are physically adsorbed onto the support.

In certain embodiments of the invention, a capture agent is immobilized on a support in ways that separate the capture agent's PET binding site region and the region where it is linked to the support. In a preferred embodiment, the capture agent is engineered to form a covalent bond between one of its termini to an adapter molecule on the support. Such a covalent bond may be formed through a Schiff-base linkage, a linkage generated by a Michael addition, or a thioether linkage.

In order to allow attachment by an adapter or directly by a capture agent, the surface of the substrate may require preparation to create suitable reactive groups. Such reactive groups could include simple chemical moieties such as amino, hydroxyl, carboxyl, carboxylate, aldehyde, ester, amide, amine, nitrile, sulfonyl, phosphoryl, or similarly chemically reactive groups. Alternatively, reactive groups may comprise more complex moieties that include, but are not limited to, sulfo-N-hydroxysuccinimide, nitrilotriacetic acid, activated hydroxyl, haloacetyl (e.g., bromoacetyl, iodoacetyl), activated carboxyl, hydrazide, epoxy, aziridine, sulfonylchloride, trifluoromethyldiaziridine, pyridyldisulfide, N-acyl-imidazole, imidazolecarbamate, succinimidylcarbonate, arylazide, anhydride, diazoacetate, benzophenone, isothiocyanate, isocyanate, imidoester, fluorobenzene, biotin and avidin. Techniques of placing such reactive groups on a substrate by mechanical, physical, electrical or chemical means are well known in the art, such as described by U.S. Pat. No. 4,681,870.

Once the initial preparation of reactive groups on the substrate is completed (if necessary), adapter molecules optionally may be added to the surface of the substrate to make it suitable for further attachment chemistry. Such adapters covalently join the reactive groups already on the substrate and the capture agents to be immobilized, having a backbone of chemical bonds forming a continuous connection between the reactive groups on the substrate and the capture agents, and having a plurality of freely rotating bonds along that backbone. Substrate adapters may be selected from any suitable class of compounds and may comprise polymers or copolymers of organic acids, aldehydes, alcohols, thiols, amines and the like. For example, polymers or copolymers of hydroxy-, amino-, or di-carboxylic acids, such as glycolic acid, lactic acid, sebacic acid, or sarcosine may be employed. Alternatively, polymers or copolymers of saturated or unsaturated hydrocarbons such as ethylene glycol, propylene glycol, saccharides, and the like may be employed. Preferably, the substrate adapter should be of an appropriate length to allow the capture agent, which is to be attached, to interact freely with molecules in a sample solution and to form effective binding. The substrate adapters may be either branched or unbranched, but this and other structural attributes of the adapter should not interfere stereochemically with relevant functions of the capture agents, such as a PET interaction. Protection groups, known to those skilled in the art, may be used to prevent the adapter's end groups from undesired or premature reactions. For instance, U.S. Pat. No. 5,412,087, describes the use of photo-removable protection groups on a adapter's thiol group.

To preserve the binding affinity of a capture agent, it is preferred that the capture agent be modified so that it binds to the support substrate at a region separate from the region responsible for interacting with it's ligand, i.e., the PET.

Methods of coupling the capture agent to the reactive end groups on the surface of the substrate or on the adapter include reactions that form linkage such as thioether bonds, disulfide bonds, amide bonds, carbamate bonds, urea linkages, ester bonds, carbonate bonds, ether bonds, hydrazone linkages, Schiff-base linkages, and noncovalent linkages mediated by, for example, ionic or hydrophobic interactions. The form of reaction will depend, of course, upon the available reactive groups on both the substrate/adapter and capture agent.

C. Array Fabrication Consideration

Preferably, the immobilized capture agents are arranged in an array on a solid support, such as a silicon-based chip or glass slide. One or more capture agents designed to detect the presence (and optionally the concentration) of a given known protein (one previously recognized as existing) is immobilized at each of a plurality of cells/regions in the array. Thus, a signal at a particular cell/region indicates the presence of a known protein in the sample, and the identity of the protein is revealed by the position of the cell. Alternatively, capture agents for one or a plurality of PET are immobilized on beads, which optionally are labeled to identify their intended target analyte, or are distributed in an array such as a microwell plate.

In one embodiment, the microarray is high density, with a density over about 100, preferably over about 1000, 1500, 2000, 3000, 4000, 5000 and further preferably over about 9000, 10000, 11000, 12000 or 13000 spots per cm², formed by attaching capture agents onto a support surface which has been functionalized to create a high density of reactive groups or which has been functionalized by the addition of a high density of adapters bearing reactive groups. In another embodiment, the microarray comprises a relatively small number of capture agents, e.g., 10 to 50, selected to detect in a sample various combinations of specific proteins which generate patterns probative of disease diagnosis, cell type determination, pathogen identification, etc.

Although the characteristics of the substrate or support may vary depending upon the intended use, the shape, material and surface modification of the substrates must be considered. Although it is preferred that the substrate have at least one surface which is substantially planar or flat, it may also include indentations, protuberances, steps, ridges, terraces and the like and may have any geometric form (e.g., cylindrical, conical, spherical, concave surface, convex surface, string, or a combination of any of these). Suitable substrate materials include, but are not limited to, glasses, ceramics, plastics, metals, alloys, carbon, papers, agarose, silica, quartz, cellulose, polyacrylamide, polyamide, and gelatin, as well as other polymer supports, other solid-material supports, or flexible membrane supports. Polymers that may be used as substrates include, but are not limited to: polystyrene; poly(tetra)fluoroethylene (PTFE); polyvinylidenedifluoride; polycarbonate; polymethylmethacrylate; polyvinylethylene; polyethyleneimine; polyoxymethylene (POM); polyvinylphenol; polylactides; polymethacrylimide (PMI); polyalkenesulfone (PAS); polypropylene; polyethylene; polyhydroxyethylmethacrylate (HEMA); polydimethylsiloxane; polyacrylamide; polyimide; and various block co-polymers. The substrate can also comprise a combination of materials, whether water-permeable or not, in multi-layer configurations. A preferred embodiment of the substrate is a plain 2.5 cm×7.5 cm glass slide with surface Si—OH functionalities.

Array fabrication methods include robotic contact printing, ink-jetting, piezoelectric spotting and photolithography. A number of commercial arrayers are available [e.g. Perkin Elmer] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

A microfluidics system for automated sample incubation with arrays on glass slides and washing has been codeveloped by NextGen and PerkinElmer Lifesciences.

For example, capture agent microarrays may be produced by a number of means, including “spotting” wherein small amounts of the reactants are dispensed to particular positions on the surface of the substrate. Methods for spotting include, but are not limited to, microfluidics printing, microstamping (see, e.g., U.S. Pat. No. 5,515,131, U.S. Pat. No. 5,731,152, Martin, B. D. et al. (1998), Langmuir 14: 3971-3975 and Haab, B B et al. (2001) Genome Biol 2 and MacBeath, G. et al. (2000) Science 289: 1760⁻1763), microcontact printing (see, e.g., PCT Publication WO 96/29629), inkjet head printing (Roda, A. et al. (2000) BioTechniques 28: 492⁻496, and Silzel, J. W. et al. (1998) Clin Chem 44: 2036-2043), microfluidic direct application (Rowe, C. A. et al. (1999) Anal Chem 71: 433-439 and Bernard, A. et al. (2001), Anal Chem 73: 8⁻12) and electrospray deposition (Morozov, V. N. et al. (1999) Anal Chem 71: 1415-1420 and Moerman R. et al. (2001) Anal Chem 73: 2183-2189). Generally, the dispensing device includes calibrating means for controlling the amount of sample deposition, and may also include a structure for moving and positioning the sample in relation to the support surface. The volume of fluid to be dispensed per capture agent in an array varies with the intended use of the array, and available equipment. Preferably, a volume formed by one dispensation is less than 100 nL, more preferably less than 10 nL, and most preferably about 1 nL. The size of the resultant spots will vary as well, and in preferred embodiments these spots are less than 20,000 μm in diameter, more preferably less than 2,000 μm in diameter, and most preferably about 150-200 μm in diameter (to yield about 1600 spots per square centimeter). Solutions of blocking agents may be applied to the microarrays to prevent non-specific binding by reactive groups that have not bound to a capture agent. Solutions of bovine serum albumin (BSA), casein, or nonfat milk, for example, may be used as blocking agents to reduce background binding in subsequent assays.

In preferred embodiments, high-precision, contact-printing robots are used to pick up small volumes of dissolved capture agents from the wells of a microtiter plate and to repetitively deliver approximately 1 nL of the solutions to defined locations on the surfaces of substrates, such as chemically-derivatized glass microscope slides. Examples of such robots include the GMS 417 Arrayer, commercially available from Affymetrix of Santa Clara, Calif., and a split pin arrayer constructed according to instructions downloadable from the Brown lab website at cmgm.stanford dot edu/pbrown. This results in the formation of microscopic spots of compounds on the slides. It will be appreciated by one of ordinary skill in the art, however, that the current invention is not limited to the delivery of 1 nL volumes of solution, to the use of particular robotic devices, or to the use of chemically derivatized glass slides, and that alternative means of delivery can be used that are capable of delivering picoliter or smaller volumes. Hence, in addition to a high precision array robot, other means for delivering the compounds can be used, including, but not limited to, ink jet printers, piezoelectric printers, and small volume pipetting robots.

In one embodiment, the compositions, e.g., microarrays or beads, comprising the capture agents of the present invention may also comprise other components, e.g., molecules that recognize and bind specific peptides, metabolites, drugs or drug candidates, RNA, DNA, lipids, and the like. Thus, an array of capture agents only some of which bind a PET can comprise an embodiment of the invention.

As an alternative to planar microarrays, bead-based assays combined with fluorescence-activated cell sorting (FACS) have been developed to perform multiplexed immunoassays. Fluorescence-activated cell sorting has been routinely used in diagnostics for more than 20 years. Using mAbs, cell surface markers are identified on normal and neoplastic cell populations enabling the classification of various forms of leukemia or disease monitoring (recently reviewed by Herzenberg et al. Immunol Today 21 (2000), pp. 383-390).

Bead-based assay systems employ microspheres as solid support for the capture molecules instead of a planar substrate, which is conventionally used for microarray assays. In each individual immunoassay, the capture agent is coupled to a distinct type of microsphere. The reaction takes place on the surface of the microspheres. The individual microspheres are color-coded by a uniform and distinct mixture of red and orange fluorescent dyes. After coupling to the appropriate capture molecule, the different color-coded bead sets can be pooled and the immunoassay is performed in a single reaction vial. Product formation of the PET targets with their respective capture agents on the different bead types can be detected with a fluorescence-based reporter system. The signal intensities are measured in a flow cytometer, which is able to quantify the amount of captured targets on each individual bead. Each bead type and thus each immobilized target is identified using the color code measured by a second fluorescence signal. This allows the multiplexed quantification of multiple targets from a single sample. Sensitivity, reliability and accuracy are similar to those observed with standard microtiter ELISA procedures. Color-coded microspheres can be used to perform up to a hundred different assay types simultaneously (LabMAP system, Laboratory Muliple Analyte Profiling, Luminex, Austin, Tex., USA). For example, microsphere-based systems have been used to simultaneously quantify cytokines or autoantibodies from biological samples (Carson and Vignali, J Immunol Methods 227 (1999), pp. 41-52; Chen et al., Clin Chem 45 (1999), pp. 1693-1694; Fulton et al., Clin Chem 43 (1997), pp. 1749-1756). Bellisario et al. (Early Hum Dev 64 (2001), pp. 21-25) have used this technology to simultaneously measure antibodies to three HIV-1 antigens from newborn dried blood-spot specimens.

Bead-based systems have several advantages. As the capture molecules are coupled to distinct microspheres, each individual coupling event can be perfectly analyzed. Thus, only quality-controlled beads can be pooled for multiplexed immunoassays. Furthermore, if an additional parameter has to be included into the assay, one must only add a new type of loaded bead. No washing steps are required when performing the assay. The sample is incubated with the different bead types together with fluorescently labeled detection antibodies. After formation of the sandwich immuno-complex, only the fluorophores that are definitely bound to the surface of the microspheres are counted in the flow cytometer.

D. Related Non-Array Formats

An alternative to an array of capture agents is one made through the so-called “molecular imprinting” technology, in which peptides (e.g. selected PETs) are used as templates to generate structurally complementary, sequence-specific cavities in a polymerisable matrix; the cavities can then specifically capture (digested) proteins which have the appropriate primary amino acid sequence [ProteinPrint™, Aspira Biosystems]. To illustrate, a chosen PET can be synthesized, and a universal matrix of polymerizable monomers is allowed to self assemble around the peptide and crosslinked into place. The PET, or template, is then removed, leaving behind a cavity complementary in shape and functionality. The cavities can be formed on a film, discrete sites of an array or the surface of beads. When a sample of fragmented proteins is exposed to the capture agent, the polymer will selectively retain the target protein containing the PET and exclude all others. After the washing, only the bound PET-containing peptides remain. Common staining and tagging procedures, or any of the non-labeling techniques described below can be used to detect expression levels and/or post translational modifications. See, for example, WO 01/61354 A1 and WO 01/61355 A1.

Alternatively, the captured peptides can be eluted for further analysis such as mass spectrometry analysis. Although several well-established chemical methods for the sequencing of peptides, polypeptides and proteins are known (for example, the Edman degradation), mass spectrometric methods are becoming increasingly important in view of their speed and ease of use. Mass spectrometric methods have been developed to the point at which they are capable of sequencing peptides in a mixture even without any prior chemical purification or separation, typically using electrospray ionization and tandem mass spectrometry (MS/MS). For example, see Yates III (J. Mass Spectrom, 1998 vol. 33 pp. 1-19), Papayannopoulos (Mass Spectrom. Rev. 1995, vol. 14 pp. 49-73), and Yates III, McCormack, and Eng (Anal. Chem. 1996 vol. 68 (17) pp. 534A-540A). Thus, in a typical MS/MS sequencing experiment, molecular ions of a particular peptide are selected by the first mass analyzer and fragmented by collisions with neutral gas molecules in a collision cell. The second mass analyzer is then used to record the fragment ion spectrum that generally contains enough information to allow at least a partial, and often the complete, sequence to be determined. See, for example, U.S. Pat. No. 6,489,608, 5,470,753, 5,246,865, and related applications/patents.

Another methodology which can be used diagnostically and in expression profiling is the ProteinChip® array [Ciphergen], in which solid phase chromatographic surfaces bind proteins with similar characteristics of charge or hydrophobicity from mixtures such as plasma or tumor extracts, and SELDI-TOF mass spectrometry is used to detection the retained proteins. The ProteinChip® is credited with the ability to identify novel disease markers. However, this technology differs from the protein arrays under discussion here since, in general, it does not involve immobilization of individual proteins for detection of specific ligand interactions.

E. Single Assay Format

PET-specific affinity capture agents can also be used in a single assay format. For example, such agents can be used to develop a better assay for detecting circulating agents, such as PSA, by providing increased sensitivity, dynamic range and/or recovery rate. For instance, the single assays can have functional performance characteristics which exceed traditional ELISA and other immunoassays, such as one or more of the following: a regression coefficient (R2) of 0.95 or greater for a reference standard, e.g., a comparable control sample, more preferably an R2 greater than 0.97, 0.99 or even 0.995; a recovery rate of at least 50 percent, and more preferably at least 60, 75, 80 or even 90 percent; a positive predictive value for occurrence of the protein in a sample of at least 90 percent, more preferably at least 95, 98 or even 99 percent; a diagnostic sensitivity (DSN) for occurrence of the protein in a sample of 99 percent or higher, more preferably at least 99.5 or even 99.8 percent; a diagnostic specificity (DSP) for occurrence of the protein in a sample of 99 percent or higher, more preferably at least 99.5 or even 99.8 percent.

### 8. Methods of Detecting Binding Events

The capture agents of the invention, as well as compositions, e.g., microarrays or beads, comprising these capture agents have a wide range of applications in the health care industry, e.g., in therapy, in clinical diagnostics, in in vivo imaging or in drug discovery. The capture agents of the present invention also have industrial and environmental applications, e.g., in environmental diagnostics, industrial diagnostics, food safety, toxicology, catalysis of reactions, or high-throughput screening; as well as applications in the agricultural industry and in basic research, e.g., protein sequencing.

The capture agents and methods of the present invention provide a powerful analytical tool that enables a user to detect a specific protein, or group of proteins of interest present within complex samples. In addition, the invention allow for efficient and rapid analysis of samples; sample conservation and direct sample comparison. The invention enables “multi-parametric” analysis of protein samples. As used herein, a “multi-parametric” analysis of a protein sample is intended to include an analysis of a protein sample based on a plurality of parameters. For example, a protein sample may be contacted with a plurality of PETs, each of the PETs being able to detect a different protein within the sample. Based on the combination and, preferably the relative concentration, of the proteins detected in the sample the skilled artisan would be able to determine the identity of a sample, diagnose a disease or pre-disposition to a disease, or determine the stage of a disease.

The capture agents of the present invention may be used in any method suitable for detection of a protein or a polypeptide, such as, for example, in immunoprecipitations, immunocytochemistry, Western Blots or nuclear magnetic resonance spectroscopy (NMR).

To detect the presence of a protein that interacts with a capture agent, a variety of art known methods may be used. The protein to be detected may be labeled with a detectable label, and the amount of bound label directly measured. The term “label” is used herein in a broad sense to refer to agents that are capable of providing a detectable signal, either directly or through interaction with one or more additional members of a signal producing system. Labels that are directly detectable and may find use in the present invention include, for example, fluorescent labels such as fluorescein, rhodamine, BODIPY, cyanine dyes (e.g. from GE), Alexa dyes (e.g. from Invitrogen (Carlsbad, Calif.), fluorescent dye phosphoramidites, beads, chemilumninescent compounds, colloidal particles, and the like. Suitable fluorescent dyes are known in the art, including fluoresceinisothiocyanate (FITC); rhodamine and rhodamine derivatives; Texas Red; phycoerythrin; allophycocyanin; 6-carboxyfluorescein (6-FAM); 2′,7′-dimethoxy-41,51-dichloro carboxyfluorescein (JOE); 6-carboxy-X-rhodamine (ROX); 6-carboxy-21,41,71,4,7-hexachlorofluorescein (HEX); 5-carboxyfluorescein (5-FAM); N,N,N1,N′-tetramethyl carboxyrhodamine (TAMRA); sulfonated rhodamine; Cy3; Cy5, etc. Radioactive isotopes, such as ³⁵S, ³²P, ³H, ¹²⁵I, etc., and the like can also be used for labeling. In addition, labels may also include near-infrared dyes (Wang et al., Anal. Chem., 72:5907-5917 (2000), upconverting phosphors (Hampl et al., Anal. Biochem., 288:176-187 (2001), DNA dendrimers (Stears et al., Physiol. Genomics 3: 93-99 (2000), quantum dots (Bruchez et al., Science 281:2013-2016 (1998), latex beads (Okana et al., Anal. Biochem. 202:120-125 (1992), selenium particles (Stimpson et al., Proc. Natl. Acad. Sci. 92:6379-6383 (1995), and europium nanoparticles (Harma et al., Clin. Chem. 47:561-568 (2001). The label is one that preferably does not provide a variable signal, but instead provides a constant and reproducible signal over a given period of time.

A very useful labeling agent is water-soluble quantum dots, or so-called “functionalized nanocrystals” or “semiconductor nanocrystals” as described in U.S. Pat. No. 6,114,038. Generally, quantum dots can be prepared which result in relative monodispersity (e.g., the diameter of the core varying approximately less than 10% between quantum dots in the preparation), as has been described previously (Bawendi et al., 1993, J. Am. Chem. Soc. 115:8706). Examples of quantum dots are known in the art to have a core selected from the group consisting of CdSe, CdS, and CdTe (collectively referred to as “CdX”)(see, e.g., Norris et al., 1996, Physical Review B. 53:16338-16346; Nirmal et al., 1996, Nature 383:802-804; Empedocles et al., 1996, Physical Review Letters 77:3873-3876; Murray et al., 1996, Science 270: 1355-1338; Effros et al., 1996, Physical Review B. 54:4843-4856; Sacra et al., 1996, J. Chem. Phys. 103:5236-5245; Murakoshi et al., 1998, J. Colloid Interface Sci. 203:225-228; Optical Materials and Engineering News, 1995, Vol. 5, No. 12; and Murray et al., 1993, J. Am. Chem. Soc. 115:8706-8714.

CdX quantum dots have been passivated with an inorganic coating (“shell”) uniformly deposited thereon. Passivating the surface of the core quantum dot can result in an increase in the quantum yield of the luminescence emission, depending on the nature of the inorganic coating. The shell which is used to passivate the quantum dot is preferably comprised of YZ wherein Y is Cd or Zn, and Z is S, or Se. Quantum dots having a CdX core and a YZ shell have been described in the art (see, e.g., Danek et al., 1996, Chem. Mater. 8:173-179; Dabbousi et al., 1997, J. Phys. Chem. B 101:9463; Rodriguez-Viejo et al., 1997, Appl. Phys. Lett. 70:2132-2134; Peng et al., 1997, J. Am. Chem. Soc. 119:7019-7029; 1996, Phys. Review B. 53:16338-16346. However, the above described quantum dots, passivated using an inorganic shell, have only been soluble in organic, non-polar (or weakly polar) solvents. To make quantum dots useful in biological applications, it is desirable that the quantum dots are water-soluble. “Water-soluble” is used herein to mean sufficiently soluble or suspendable in an aqueous-based solution, such as in water or water-based solutions or buffer solutions, including those used in biological or molecular detection systems as known by those skilled in the art.

U.S. Pat. No. 6,114,038 provides a composition comprising functionalized nanocrystals for use in non-isotopic detection systems. The composition comprises quantum dots (capped with a layer of a capping compound) that are water-soluble and functionalized by operably linking, in a successive manner, one or more additional compounds. In a preferred embodiment, the one or more additional compounds form successive layers over the nanocrystal. More particularly, the functionalized nanocrystals comprise quantum dots capped with the capping compound, and have at least a diaminocarboxylic acid which is operatively linked to the capping compound. Thus, the functionalized nanocrystals may have a first layer comprising the capping compound, and a second layer comprising a diaminocarboxylic acid; and may further comprise one or more successive layers including a layer of amino acid, a layer of affinity ligand, or multiple layers comprising a combination thereof. The composition comprises a class of quantum dots that can be excited with a single wavelength of light resulting in detectable luminescence emissions of high quantum yield and with discrete luminescence peaks. Such functionalized nanocrystal may be used to label capture agents of the instant invention for their use in the detection and/or quantitation of the binding events.

U.S. Pat. No. 6,326,144 describes quantum dots (QDs) having a characteristic spectral emission, which is tunable to a desired energy by selection of the particle size of the quantum dot. For example, a 2 nanometer quantum dot emits green light, while a 5 nanometer quantum dot emits red light. The emission spectra of quantum dots have linewidths as narrow as 25-30 nm depending on the size heterogeneity of the sample, and lineshapes that are symmetric, gaussian or nearly gaussian with an absence of a tailing region. The combination of tunability, narrow linewidths, and symmetric emission spectra without a tailing region provides for high resolution of multiply-sized quantum dots within a system and enables researchers to examine simultaneously a variety of biological moieties tagged with QDs. In addition, the range of excitation wavelengths of the nanocrystal quantum dots is broad and can be higher in energy than the emission wavelengths of all available quantum dots. Consequently, this allows the simultaneous excitation of all quantum dots in a system with a single light source, usually in the ultraviolet or blue region of the spectrum. QDs are also more robust than conventional organic fluorescent dyes and are more resistant to photobleaching than the organic dyes. The robustness of the QD also alleviates the problem of contamination of the degradation products of the organic dyes in the system being examined. These QDs can be used for labeling capture agents of protein, nucleic acid, and other biological molecules in nature. Cadmium Selenide quantum dot nanocrystals are available from Quantum Dot Corporation of Hayward, Calif.

Alternatively, the sample to be tested is not labeled, but a second stage labeled reagent is added in order to detect the presence or quantitate the amount of protein in the sample. Such “sandwich based” methods of detection have the requirement that two capture agents must be developed for each protein, one to capture the PET and one to label it once captured. Such methods have the advantage that they are characterized by an inherently improved signal to noise ratio as they exploit two binding reactions at different points on a peptide, thus the presence and/or concentration of the protein can be measured with more accuracy and precision because of the increased signal to noise ratio.

In yet another embodiment, the subject capture array can be a “virtual arrays”. For example, a virtual array can be generated in which antibodies or other capture agents are immobilized on beads whose identity, with respect to the particular PET it is specific for as a consequence to the associated capture agent, is encoded by a particular ratio of two or more covalently attached dyes. Mixtures of encoded PET-beads are added to a sample, resulting in capture of the PET entities recognized by the immobilized capture agents.

To quantitate the captured species, a sandwich assay with fluorescently labeled antibodies that bind the captured PET, or a competitive binding assay with a fluorescently labeled ligand for the capture agent, are added to the mix. In one embodiment, the labeled ligand is a labeled PET that competes with the analyte PET for binding to the capture agent. The beads are then introduced into an instrument, such as a flow cytometer, that reads the intensity of the various fluorescence signals on each bead, and the identity of the bead can be determined by measuring the ratio of the dyes. This technology is relatively fast and efficient, and can be adapted by researchers to monitor almost any set of PET of interest.

In another embodiment, an array of capture agents are embedded in a matrix suitable for ionization (such as described in Fung et al. (2001) Curr. Opin. Biotechnol. 12:65-69). After application of the sample and removal of unbound molecules (by washing), the retained PET proteins are analyzed by mass spectrometry. In some instances, further proteolytic digestion of the bound species with trypsin may be required before ionization, particularly if electrospray is the means for ionizing the peptides.

All the above named reagents may be used to label the capture agents. Preferably, the capture agent to be labeled is combined with an activated dye that reacts with a group present on the protein to be detected, e.g., amine groups, thiol groups, or aldehyde groups.

The label may also be a covalently bound enzyme capable of providing a detectable product signal after addition of suitable substrate. Examples of suitable enzymes for use in the present invention include horseradish peroxidase, alkaline phosphatase, malate dehydrogenase and the like.

Enzyme-Linked Immunosorbent Assay (ELISA) may also be used for detection of a protein that interacts with a capture agent. In an ELISA, the indicator molecule is covalently coupled to an enzyme and may be quantified by determining with a spectrophotometer the initial rate at which the enzyme converts a clear substrate to a correlated product. Methods for performing ELISA are well known in the art and described in, for example, Perlmann, H. and Perlmann, P. (1994). Enzyme-Linked Immunosorbent Assay. In: Cell Biology: A Laboratory Handbook. San Diego, Calif., Academic Press, Inc., 322-328; Crowther, J. R. (1995). Methods in Molecular Biology, Vol. 42-ELISA: Theory and Practice. Humana Press, Totowa, N.J.; and Harlow, E. and Lane, D. (1988). Antibodies: A Laboratory Manual. Cold Spring Harbor Laboratory Press, Cold Spring Harbor, N.Y., 553-612. Sandwich (capture) ELISA may also be used to detect a protein that interacts with two capture agents. The two capture agents may be able to specifically interact with two PETs that are present on the same peptide (e.g., the peptide which has been generated by fragmentation of the sample of interest, as described above). Alternatively, the two capture agents may be able to specifically interact with one PET and one non-unique amino acid sequence, both present on the same peptide (e.g., the peptide which has been generated by fragmentation of the sample of interest, as described above). Sandwich ELISAs for the quantitation of proteins of interest are especially valuable when the concentration of the protein in the sample is low and/or the protein of interest is present in a sample that contains high concentrations of contaminating proteins.

A fully-automated, microarray-based approach for high-throughput, ELISAs was described by Mendoza et al. (BioTechniques 27:778-780,782-786,788, 1999). This system consisted of an optically flat glass plate with 96 wells separated by a Teflon mask. More than a hundred capture molecules were immobilized in each well. Sample incubation, washing and fluorescence-based detection were performed with an automated liquid pipettor. The microarrays were quantitatively imaged with a scanning charge-coupled device (CCD) detector. Thus, the feasibility of multiplex detection of arrayed antigens in a high-throughput fashion using marker antigens could be successfully demonstrated. In addition, Silzel et al. (Clin Chem 44 pp. 2036-2043, 1998) could demonstrate that multiple IgG subclasses can be detected simultaneously using microarray technology. Wiese et al. (Clin Chem 47 pp. 1451-1457, 2001) were able to measure prostate-specific antigen (PSA), -(1)-antichymotrypsin-bound PSA and interleukin-6 in a microarray format. Arenkov et al. (supra) carried out microarray sandwich immunoassays and direct antigen or antibody detection experiments using a modified polyacrylamide gel as substrate for immobilized capture molecules.

Most of the microarray assay formats described in the art rely on chemiluminescence- or fluorescence-based detection methods. A further improvement with regard to sensitivity involves the application of fluorescent labels and waveguide technology. A fluorescence-based array immunosensor was developed by Rowe et al. (Anal Chem 71 (1999), pp. 433-439; and Biosens Bioelectron 15 (2000), pp. 579-589) and applied for the simultaneous detection of clinical analytes using the sandwich immunoassay format. Biotinylated capture antibodies were immobilized on avidin-coated waveguides using a flow-chamber module system. Discrete regions of capture molecules were vertically arranged on the surface of the waveguide. Samples of interest were incubated to allow the targets to bind to their capture molecules. Captured targets were then visualized with appropriate fluorescently labeled detection molecules. This array immunosensor was shown to be appropriate for the detection and measurement of targets at physiologically relevant concentrations in a variety of clinical samples.

A further increase in the sensitivity using waveguide technology was achieved with the development of the planar waveguide technology (Duveneck et al., Sens Actuators B B38 (1997), pp. 88-95). Thin-film waveguides are generated from a high-refractive material such as Ta₂O₅ that is deposited on a transparent substrate. Laser light of desired wavelength is coupled to the planar waveguide by means of diffractive grating. The light propagates in the planar waveguide and an area of more than a square centimeter can be homogeneously illuminated. At the surface, the propagating light generates a so-called evanescent field. This extends into the solution and activates only fluorophores that are bound to the surface. Fluorophores in the surrounding solution are not excited. Close to the surface, the excitation field intensities can be a hundred times higher than those achieved with standard confocal excitation. A CCD camera is used to identify signals simultaneously across the entire area of the planar waveguide. Thus, the immobilization of the capture molecules in a microarray format on the planar waveguide allows the performance of highly sensitive miniaturized and parallelized immunoassays. This system was successfully employed to detect interleukin-6 at concentrations as low as 40 fM and has the additional advantage that the assay can be performed without washing steps that are usually required to remove unbound detection molecules (Weinberger et al., Pharmacogenomics 1: 395-416, 2000).

Alternative strategies pursued to increase sensitivity are based on signal amplification procedures. For example, immunoRCA (immuno rolling circle amplification) involves an oligonucleotide primer that is covalently attached to a detection molecule (such as a second capture agent in a sandwich-type assay format). Using circular DNA as template, which is complementary to the attached oligonucleotide, DNA polymerase will extend the attached oligonucleotide and generate a long DNA molecule consisting of hundreds of copies of the circular DNA, which remains attached to the detection molecule. The incorporation of thousands of fluorescently labeled nucleotides will generate a strong signal. Schweitzer et al. (Proc Natl Acad Sci USA 97 (2000), pp. 10113-10119) have evaluated this detection technology for use in microarray-based assays. Sandwich immunoassays for huIgE and prostate-specific antigens were performed in a microarray format. The antigens could be detected at femtomolar concentrations and it was possible to score single, specifically captured antigens by counting discrete fluorescent signals that arose from the individual antibody-antigen complexes. The authors demonstrated that immunoassays employing rolling circle DNA amplification are a versatile platform for the ultra-sensitive detection of antigens and thus are well suited for use in protein microarray technology.

A novel technology for protein detection, proximity ligation, has recently been developed, along with improved methods for in situ synthesis of DNA microarrays. Proximity ligation may be another amplification strategy that can be employed with anti-PET antibodies. Proximity ligation enables a specific and quantitative transformation of proteins present in a sample into nucleic acid sequences. As pairs of so-called proximity probes bind the individual target molecules at distinct sites (say two adjacent epitopes on the same target molecule), these proximity probes are brought in close proximity. The probes consist of a protein specific binding part coupled to an oligonucleotide with either a free 3′- or 5′-end capable of hybridizing to a common connector oligonucleotide. When the probes are in proximity, promoted by target binding, the polynucleotide strands can be joined by enzymatic ligation. The nucleic acid sequence that is formed can then be amplified and quantitatively detected in a real-time monitored polymerase chain reaction or any type of polynucleotide amplification method (such as rolling circle amplification, etc.). In certain embodiments, the common connector oligonucleotide may be omitted, and the ends of the oligonucleotides on the proximity probes may be directly ligated by, for example, T4 DNA ligase. This convenient assay is simple to perform and allows highly sensitive protein detection. It also eliminates or significantly reduces background issue associated with the immuno-PCR method (Sano et al., Chemtech January 1995, pp 24-30), where non-specifically bound oligonucleotides may also be accidentally amplified by the very sensitive PCR method. See WO 97/00446, WO 01/61037 and WO 03/044231.

In certain embodiments, immuno-PCR method such as those described in Sano et al., Chemtech January 1995, pp 24-30 may be used to detect any capture agents (e.g. Ab) that specifically bind the immobilized target analytes.

Radioimmunoassays (RIA) may also be used for detection of a protein that interacts with a capture agent. In a RIA, the indicator molecule is labeled with a radioisotope and it may be quantified by counting radioactive decay events in a scintillation counter. Methods for performing direct or competitive RIA are well known in the art and described in, for example, Cell Biology: A Laboratory Handbook. San Diego, Calif., Academic Press, Inc.

Other immunoassays commonly used to quantitate the levels of proteins in cell samples, and are well-known in the art, can be adapted for use in the instant invention. The invention is not limited to a particular assay procedure, and therefore is intended to include both homogeneous and heterogeneous procedures. Exemplary other immunoassays which can be conducted according to the invention include fluorescence polarization immunoassay (FPIA), fluorescence immunoassay (FIA), enzyme immunoassay (EIA), nephelometric inhibition immunoassay (NIA). An indicator moiety, or label group, can be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art. In one embodiment, the determination of protein level in a biological sample may be performed by a microarray analysis (protein chip).

In several other embodiments, detection of the presence of a protein that interacts with a capture agent may be achieved without labeling. For example, determining the ability of a protein to bind to a capture agent can be accomplished using a technology such as real-time Biomolecular Interaction Analysis (BIA). Sjolander and Urbaniczky, Anal. Chem. 63: 2338-45, 1991 and Szabo et al., Curr. Opin. Struct. Biol. 5: 699-705, 1995. As used herein, “BIA” is a technology for study-ing biospecific interactions in real time, without labeling any of the interactants (e.g., BIAcore).

In another embodiment, a biosensor with a special diffractive grating surface may be used to detect/quantitate binding between non-labeled PET-containing peptides in a treated (digested) biological sample and immobilized capture agents at the surface of the biosensor. Details of the technology is described in more detail in B. Cunningham, P. Li, B. Lin, J. Pepper, “Colorimetric resonant reflection as a direct biochemical assay technique,” Sensors and Actuators B, Volume 81, p. 316-328, Jan. 5, 2002, and in PCT No. WO 02/061429 A2 and US 2003/0032039. Briefly, a guided mode resonant phenomenon is used to produce an optical structure that, when illuminated with collimated white light, is designed to reflect only a single wavelength (color). When molecules are attached to the surface of the biosensor, the reflected wavelength (color) is shifted due to the change of the optical path of light that is coupled into the grating. By linking receptor molecules to the grating surface, complementary binding molecules can be detected/quantitated without the use of any kind of fluorescent probe or particle label. The spectral shifts may be analyzed to determine the expression data provided, and to indicate the presence or absence of a particular indication.

The biosensor typically comprises: a two-dimensional grating comprised of a material having a high refractive index, a substrate layer that supports the two-dimensional grating, and one or more detection probes immobilized on the surface of the two-dimensional grating opposite of the substrate layer. When the biosensor is illuminated a resonant grating effect is produced on the reflected radiation spectrum. The depth and period of the two-dimensional grating are less than the wavelength of the resonant grating effect.

A narrow band of optical wavelengths can be reflected from the biosensor when it is illuminated with a broad band of optical wavelengths. The substrate can comprise glass, plastic or epoxy. The two-dimensional grating can comprise a material selected from the group consisting of zinc sulfide, titanium dioxide, tantalum oxide, and silicon nitride.

The substrate and two-dimensional grating can optionally comprise a single unit. The surface of the single unit comprising the two-dimensional grating is coated with a material having a high refractive index, and the one or more detection probes are immobilized on the surface of the material having a high refractive index opposite of the single unit. The single unit can be comprised of a material selected from the group consisting of glass, plastic, and epoxy.

The biosensor can optionally comprise a cover layer on the surface of the two-dimensional grating opposite of the substrate layer. The one or more detection probes are immobilized on the surface of the cover layer opposite of the two-dimensional grating. The cover layer can comprise a material that has a lower refractive index than the high refractive index material of the two-dimensional grating. For example, a cover layer can comprise glass, epoxy, and plastic.

A two-dimensional grating can be comprised of a repeating pattern of shapes selected from the group consisting of lines, squares, circles, ellipses, triangles, trapezoids, sinusoidal waves, ovals, rectangles, and hexagons. The repeating pattern of shapes can be arranged in a linear grid, i.e., a grid of parallel lines, a rectangular grid, or a hexagonal grid. The two-dimensional grating can have a period of about 0.01 microns to about 1 micron and a depth of about 0.01-1 μm.

To illustrate, biochemical interactions occurring on a surface of a calorimetric resonant optical biosensor embedded into a surface of a microarray slide, microtiter plate or other device, can be directly detected and measured on the sensor's surface without the use of fluorescent tags or calorimetric labels. The sensor surface contains an optical structure that, when illuminated with collimated white light, is designed to reflect only a narrow band of wavelengths (color). The narrow wavelength is described as a wavelength “peak.” The “peak wavelength value” (PWV) changes when biological material is deposited or removed from the sensor surface, such as when binding occurs. Such binding-induced change of PWV can be measured using a measurement instrument disclosed in US2003/0032039.

In one embodiment, the instrument illuminates the biosensor surface by directing a collimated white light on to the sensor structure. The illuminated light may take the form of a spot of collimated light. Alternatively, the light is generated in the form of a fan beam. The instrument collects light reflected from the illuminated biosensor surface. The instrument may gather this reflected light from multiple locations on the biosensor surface simultaneously. The instrument can include a plurality of illumination probes that direct the light to a discrete number of positions across the biosensor surface. The instrument measures the Peak Wavelength Values (PWVs) of separate locations within the biosensor-embedded microtiter plate using a spectrometer. In one embodiment, the spectrometer is a single-point spectrometer. Alternatively, an imaging spectrometer is used. The spectrometer can produce a PWV image map of the sensor surface. In one embodiment, the measuring instrument spatially resolves PWV images with less than 200 micron resolution.

In one embodiment, a subwavelength structured surface (SWS) may be used to create a sharp optical resonant reflection at a particular wavelength that can be used to track with high sensitivity the interaction of biological materials, such as specific binding substances or binding partners or both. A colormetric resonant diffractive grating surface acts as a surface binding platform for specific binding substances (such as immobilized capture agents of the instant invention). SWS is an unconventional type of diffractive optic that can mimic the effect of thin-film coatings. (Peng & Morris, “Resonant scattering from two-dimensional gratings,” J. Opt. Soc. Am. A, Vol. 13, No. 5, p. 993, May; Magnusson, & Wang, “New principle for optical filters,” Appl. Phys. Lett., 61, No. 9, p. 1022, August, 1992; Peng & Morris, “Experimental demonstration of resonant anomalies in diffraction from two-dimensional gratings,” Optics Letters, Vol. 21, No. 8, p. 549, April 1996). A SWS structure contains a surface-relief, two-dimensional grating in which the grating period is small compared to the wavelength of incident light so that no diffractive orders other than the reflected and transmitted zeroth orders are allowed to propagate. A SWS surface narrowband filter can comprise a two-dimensional grating sandwiched between a substrate layer and a cover layer that fills the grating grooves. Optionally, a cover layer is not used. When the effective index of refraction of the grating region is greater than the substrate or the cover layer, a waveguide is created. When a filter is designed accordingly, incident light passes into the waveguide region. A two-dimensional grating structure selectively couples light at a narrow band of wavelengths into the waveguide. The light propagates only a short distance (on the order of 10-100 micrometers), undergoes scattering, and couples with the forward- and backward-propagating zeroth-order light. This sensitive coupling condition can produce a resonant grating effect on the reflected radiation spectrum, resulting in a narrow band of reflected or transmitted wavelengths (colors). The depth and period of the two-dimensional grating are less than the wavelength of the resonant grating effect.

The reflected or transmitted color of this structure can be modulated by the addition of molecules such as capture agents or their PET-containing binding partners or both, to the upper surface of the cover layer or the two-dimensional grating surface. The added molecules increase the optical path length of incident radiation through the structure, and thus modify the wavelength (color) at which maximum reflectance or transmittance will occur. Thus in one embodiment, a biosensor, when illuminated with white light, is designed to reflect only a single wavelength. When specific binding substances are attached to the surface of the biosensor, the reflected wavelength (color) is shifted due to the change of the optical path of light that is coupled into the grating. By linking specific binding substances to a biosensor surface, complementary binding partner molecules can be detected without the use of any kind of fluorescent probe or particle label. The detection technique is capable of resolving changes of, for example, about 0.1 nm thickness of protein binding, and can be performed with the biosensor surface either immersed in fluid or dried. This PWV change can be detected by a detection system consists of, for example, a light source that illuminates a small spot of a biosensor at normal incidence through, for example, a fiber optic probe. A spectrometer collects the reflected light through, for example, a second fiber optic probe also at normal incidence. Because no physical contact occurs between the excitation/detection system and the biosensor surface, no special coupling prisms are required. The biosensor can, therefore, be adapted to a commonly used assay platform including, for example, microtiter plates and microarray slides. A spectrometer reading can be performed in several milliseconds, thus it is possible to efficiently measure a large number of molecular interactions taking place in parallel upon a biosensor surface, and to monitor reaction kinetics in real time.

Various embodiments, variations of the biosensor described above can be found in US2003/0032039.

One or more specific capture agents may be immobilized on the two-dimensional grating or cover layer, if present. Immobilization may occur by any of the above described methods. Suitable capture agents can be, for example, a nucleic acid, polypeptide, antigen, polyclonal antibody, monoclonal antibody, single chain antibody (scFv), F(ab) fragment, F(ab′)2 fragment, Fv fragment, small organic molecule, even cell, virus, or bacteria. A biological sample can be obtained and/or deribed from, for example, blood, plasma, serum, gastrointestinal secretions, homogenates of tissues or tumors, synovial fluid, feces, saliva, sputum, cyst fluid, amniotic fluid, cerebrospinal fluid, peritoneal fluid, lung lavage fluid, semen, lymphatic fluid, tears, or prostatitc fluid. Preferably, one or more specific capture agents are arranged in a microarray of distinct locations on a biosensor. A microarray of capture agents comprises one or more specific capture agents on a surface of a biosensor such that a biosensor surface contains a plurality of distinct locations, each with a different capture agent or with a different amount of a specific capture agent. For example, an array can comprise 1, 10, 100, 1,000, 10,000, or 100,000 distinct locations. A biosensor surface with a large number of distinct locations is called a microarray because one or more specific capture agents are typically laid out in a regular grid pattern in x-y coordinates. However, a microarray can comprise one or more specific capture agents laid out in a regular or irregular pattern.

A microarray spot can range from about 50 to about 500 microns in diameter. Alternatively, a microarray spot can range from about 150 to about 200 microns in diameter. One or more specific capture agents can be bound to their specific PET-containing binding partners.

In one biosensor embodiment, a microarray on a biosensor is created by placing microdroplets of one or more specific capture agents onto, for example, an x-y grid of locations on a two-dimensional grating or cover layer surface. When the biosensor is exposed to a test sample comprising one or more PET binding partners, the binding partners will be preferentially attracted to distinct locations on the microarray that comprise capture agents that have high affinity for the PET binding partners. Some of the distinct locations will gather binding partners onto their surface, while other locations will not. Thus a specific capture agent specifically binds to its PET binding partner, but does not substantially bind other PET binding partners added to the surface of a biosensor. In an alternative embodiment, a nucleic acid microarray (such as an aptamer array) is provided, in which each distinct location within the array contains a different aptamer capture agent. By application of specific capture agents with a microarray spotter onto a biosensor, specific binding substance densities of 10,000 specific binding substances/in² can be obtained. By focusing an illumination beam of a fiber optic probe to interrogate a single microarray location, a biosensor can be used as a label-free microarray readout system.

For the detection of PET binding partners at concentrations of less than about 0.1 ng/ml, one may amplify and transduce binding partners bound to a biosensor into an additional layer on the biosensor surface. The increased mass deposited on the biosensor can be detected as a consequence of increased optical path length. By incorporating greater mass onto a biosensor surface, an optical density of binding partners on the surface is also increased, thus rendering a greater resonant wavelength shift than would occur without the added mass. The addition of mass can be accomplished, for example, enzymatically, through a “sandwich” assay, or by direct application of mass (such as a second capture agent specific for the PET peptide) to the biosensor surface in the form of appropriately conjugated beads or polymers of various size and composition. Since the capture agents are PET-specific, multiple capture agents of different types and specificity can be added together to the captured PETs. This principle has been exploited for other types of optical biosensors to demonstrate sensitivity increases over 1500× beyond sensitivity limits achieved without mass amplification. See, e.g., Jenison et al., “Interference-based detection of nucleic acid targets on optically coated silicon,” Nat. Biotech. 19: 62-65, 2001.

In an alternative embodiment, a biosensor comprises volume surface-relief volume diffractive structures (a SRVD biosensor). SRVD biosensors have a surface that reflects predominantly at a particular narrow band of optical wavelengths when illuminated with a broad band of optical wavelengths. Where specific capture agents and/or PET binding partners are immobilized on a SRVD biosensor, the reflected wavelength of light is shifted. One-dimensional surfaces, such as thin film interference filters and Bragg reflectors, can select a narrow range of reflected or transmitted wavelengths from a broadband excitation source. However, the deposition of additional material, such as specific capture agents and/or PET binding partners onto their upper surface results only in a change in the resonance linewidth, rather than the resonance wavelength. In contrast, SRVD biosensors have the ability to alter the reflected wavelength with the addition of material, such as specific capture agents and/or binding partners to the surface.

A SRVD biosensor comprises a sheet material having a first and second surface. The first surface of the sheet material defines relief volume diffraction structures. Sheet material can comprise, for example, plastic, glass, semiconductor wafer, or metal film. A relief volume diffractive structure can be, for example, a two-dimensional grating, as described above, or a three-dimensional surface-relief volume diffractive grating. The depth and period of relief volume diffraction structures are less than the resonance wavelength of light reflected from a biosensor. A three-dimensional surface-relief volume diffractive grating can be, for example, a three-dimensional phase-quantized terraced surface relief pattern whose groove pattern resembles a stepped pyramid. When such a grating is illuminated by a beam of broadband radiation, light will be coherently reflected from the equally spaced terraces at a wavelength given by twice the step spacing times the index of refraction of the surrounding medium. Light of a given wavelength is resonantly diffracted or reflected from the steps that are a half-wavelength apart, and with a bandwidth that is inversely proportional to the number of steps. The reflected or diffracted color can be controlled by the deposition of a dielectric layer so that a new wavelength is selected, depending on the index of refraction of the coating.

A stepped-phase structure can be produced first in photoresist by coherently exposing a thin photoresist film to three laser beams, as described previously. See e.g., Cowen, “The recording and large scale replication of crossed holographic grating arrays using multiple beam interferometry,” in International Conference on the Application, Theory, and Fabrication of Periodic Structures, Diffraction Gratings, and Moire Phenomena II, Lerner, ed., Proc. Soc. Photo-Opt. Instrum. Eng., 503, 120-129, 1984; Cowen, “Holographic honeycomb microlens,” Opt. Eng. 24, 796-802 (1985); Cowen & Slafer, “The recording and replication of holographic micropatterns for the ordering of photographic emulsion grains in film systems,” J Imaging Sci. 31, 100-107, 1987. The nonlinear etching characteristics of photoresist are used to develop the exposed film to create a three-dimensional relief pattern. The photoresist structure is then replicated using standard embossing procedures. For example, a thin silver film may be deposited over the photoresist structure to form a conducting layer upon which a thick film of nickel can be electroplated. The nickel “master” plate is then used to emboss directly into a plastic film, such as vinyl, that has been softened by heating or solvent. A theory describing the design and fabrication of three-dimensional phase-quantized terraced surface relief pattern that resemble stepped pyramids is described: Cowen, “Aztec surface-relief volume diffractive structure,” J. Opt. Soc. Am. A, 7:1529 (1990). An example of a three-dimensional phase-quantized terraced surface relief pattern may be a pattern that resembles a stepped pyramid. Each inverted pyramid is approximately 1 micron in diameter. Preferably, each inverted pyramid can be about 0.5 to about 5 microns diameter, including for example, about 1 micron. The pyramid structures can be close-packed so that a typical microarray spot with a diameter of 150-200 microns can incorporate several hundred stepped pyramid structures. The relief volume diffraction structures have a period of about 0.1 to about 1 micron and a depth of about 0.1 to about 1 micron.

One or more specific binding substances, as described above, are immobilized on the reflective material of a SRVD biosensor. One or more specific binding substances can be arranged in microarray of distinct locations, as described above, on the reflective material.

A SRVD biosensor reflects light predominantly at a first single optical wavelength when illuminated with a broad band of optical wavelengths, and reflects light at a second single optical wavelength when one or more specific binding substances are immobilized on the reflective surface. The reflection at the second optical wavelength results from optical interference. A SRVD biosensor also reflects light at a third single optical wavelength when the one or more specific capture agents are bound to their respective PET binding partners, due to optical interference. Readout of the reflected color can be performed serially by focusing a microscope objective onto individual microarray spots and reading the reflected spectrum with the aid of a spectrograph or imaging spectrometer, or in parallel by, for example, projecting the reflected image of the microarray onto an imaging spectrometer incorporating a high resolution color CCD camera.

A SRVD biosensor can be manufactured by, for example, producing a metal master plate, and stamping a relief volume diffractive structure into, for example, a plastic material like vinyl. After stamping, the surface is made reflective by blanket deposition of, for example, a thin metal film such as gold, silver, or aluminum. Compared to MEMS-based biosensors that rely upon photolithography, etching, and wafer bonding procedures, the manufacture of a SRVD biosensor is very inexpensive.

A SWS or SRVD biosensor embodiment can comprise an inner surface. In one preferred embodiment, such an inner surface is a bottom surface of a liquid-containing vessel. A liquid-containing vessel can be, for example, a microtiter plate well, a test tube, a petri dish, or a microfluidic channel. In one embodiment, a SWS or SRVD biosensor is incorporated into a microtiter plate. For example, a SWS biosensor or SRVD biosensor can be incorporated into the bottom surface of a microtiter plate by assembling the walls of the reaction vessels over the resonant reflection surface, so that each reaction “spot” can be exposed to a distinct test sample. Therefore, each individual microtiter plate well can act as a separate reaction vessel. Separate chemical reactions can, therefore, occur within adjacent wells without intermixing reaction fluids and chemically distinct test solutions can be applied to individual wells.

This technology is useful in applications where large numbers of biomolecular interactions are measured in parallel, particularly when molecular labels would alter or inhibit the functionality of the molecules under study. High-throughput screening of pharmaceutical compound libraries with protein targets, and microarray screening of protein-protein interactions for proteomics are examples of applications that require the sensitivity and throughput afforded by the compositions and methods of the invention.

Unlike surface plasmon resonance, resonant mirrors, and waveguide biosensors, the described compositions and methods enable many thousands of individual binding reactions to take place simultaneously upon the biosensor surface. This technology is useful in applications where large numbers of biomolecular interactions are measured in parallel (such as in an array), particularly when molecular labels alter or inhibit the functionality of the molecules under study. These biosensors are especially suited for high-throughput screening of pharmaceutical compound libraries with protein targets, and microarray screening of protein-protein interactions for proteomics. A biosensor of the invention can be manufactured, for example, in large areas using a plastic embossing process, and thus can be inexpensively incorporated into common disposable laboratory assay platforms such as microtiter plates and microarray slides.

Other similar biosensors may also be used in the instant invention. Numerous biosensors have been developed to detect a variety of biomolecular complexes including oligonucleotides, antibody-antigen interactions, hormone-receptor interactions, and enzyme-substrate interactions. In general, these biosensors consist of two components: a highly specific recognition element and a transducer that converts the molecular recognition event into a quantifiable signal. Signal transduction has been accomplished by many methods, including fluorescence, interferometry (Jenison et al., “Interference-based detection of nucleic acid targets on optically coated silicon,” Nature Biotechnology, 19, p. 62-65; Lin et al., “A porous silicon-based optical interferometric biosensor,” Science, 278, p. 840-843, 1997), and gravimetry (A. Cunningham, Bioanalytical Sensors, John Wiley & Sons (1998)). Of the optically-based transduction methods, direct methods that do not require labeling of analytes with fluorescent compounds are of interest due to the relative assay simplicity and ability to study the interaction of small molecules and proteins that are not readily labeled.

These direct optical methods include surface plasmon resonance (SPR) (Jordan & Corn, “Surface Plasmon Resonance Imaging Measurements of Electrostatic Biopolymer Adsorption onto Chemically Modified Gold Surfaces,” Anal. Chem., 69:1449-1456 (1997); plasmom-resonant particles (PRPs) (Schultz et al., Proc. Nat. Acad. Sci., 97: 996-1001 (2000); grating couplers (Morhard et al., “Immobilization of antibodies in micropatterns for cell detection by optical diffraction,” Sensors and Actuators B, 70, p. 232-242, 2000); ellipsometry (Jin et al., “A biosensor concept based on imaging ellipsometry for visualization of biomolecular interactions,” Analytical Biochemistry, 232, p. 69-72, 1995), evanascent wave devices (Huber et al., “Direct optical immunosensing (sensitivity and selectivity),” Sensors and Actuators B, 6, p. 122.126, 1992), resonance light scattering (Bao et al., Anal. Chem., 74:1792-1797 (2002), and reflectometry (Brecht & Gauglitz, “Optical probes and transducers,” Biosensors and Bioelectronics, 10, p. 923-936, 1995). Changes in the optical phenomenon of surface plasmon resonance (SPR) can be used as an indication of real-time reactions between biological molecules. Theoretically predicted detection limits of these detection methods have been determined and experimentally confirmed to be feasible down to diagnostically relevant concentration ranges.

Surface plasmon resonance (SPR) has been successfully incorporated into an immunosensor format for the simple, rapid, and nonlabeled assay of various biochemical analytes. Proteins, complex conjugates, toxins, allergens, drugs, and pesticides can be determined directly using either natural antibodies or synthetic receptors with high sensitivity and selectivity as the sensing element. Immunosensors are capable of real-time monitoring of the antigen-antibody reaction. A wide range of molecules can be detected with lower limits ranging between 10⁻⁹ and 10⁻¹³ mol/L. Several successful commercial developments of SPR immunosensors are available and their web pages are rich in technical information. Wayne et al. (Methods 22: 77-91, 2000) reviewed and highlighted many recent developments in SPR-based immunoassay, functionalizations of the gold surface, novel receptors in molecular recognition, and advanced techniques for sensitivity enhancement.

Utilization of the optical phenomenon surface plasmon resonance (SPR) has seen extensive growth since its initial observation by Wood in 1902 (Phil. Mag. 4 (1902), pp. 396-402). SPR is a simple and direct sensing technique that can be used to probe refractive index (η) changes that occur in the very close vicinity of a thin metal film surface (Otto Z. Phys. 216 (1968), p. 398). The sensing mechanism exploits the properties of an evanescent field generated at the site of total internal reflection. This field penetrates into the metal film, with exponentially decreasing amplitude from the glass-metal interface. Surface plasmons, which oscillate and propagate along the upper surface of the metal film, absorb some of the plane-polarized light energy from this evanescent field to change the total internal reflection light intensity Iᵣ. A plot of Iᵣ versus incidence (or reflection) angle θ produces an angular intensity profile that exhibits a sharp dip. The exact location of the dip minimum (or the SPR angle θᵣ) can be determined by using a polynomial algorithm to fit the Iᵣ signals from a few diodes close to the minimum. The binding of molecules on the upper metal surface causes a change in η of the surface medium that can be observed as a shift in θᵣ.

The potential of SPR for biosensor purposes was realized in 1982-1983 by Liedberg et al., who adsorbed an immunoglobulin G (IgG) antibody overlayer on the gold sensing film, resulting in the subsequent selective binding and detection of IgG (Nylander et al., Sens. Actuators 3 (1982), pp. 79-84; Liedberg et al., Sens. Actuators 4 (1983), pp. 229-304). The principles of SPR as a biosensing technique have been reviewed previously (Daniels et al., Sens. Actuators 15 (1988), pp. 11-18; VanderNoot and Lai, Spectroscopy 6 (1991), pp. 28-33; Lundström Biosens. Bioelectron. 9 (1994), pp. 725-736; Liedberg et al., Biosens. Bioelectron. 10 (1995); Morgan et al., Clin. Chem. 42 (1996), pp. 193-209; Tapuchi et al., S. Afr. J. Chem. 49 (1996), pp. 8-25). Applications of SPR to biosensing were demonstrated for a wide range of molecules, from virus particles to sex hormone-binding globulin and syphilis. Most importantly, SPR has an inherent advantage over other types of biosensors in its versatility and capability of monitoring binding interactions without the need for fluorescence or radioisotope labeling of the biomolecules. This approach has also shown promise in the real-time determination of concentration, kinetic constant, and binding specificity of individual biomolecular interaction steps. Antibody-antigen interactions, peptide/protein-protein interactions, DNA hybridization conditions, biocompatibility studies of polymers, biomolecule-cell receptor interactions, and DNA/receptor-ligand interactions can all be analyzed (Pathak and Savelkoul, Immunol. Today 18 (1997), pp. 464-467). Commercially, the use of SPR-based immunoassay has been promoted by companies such as Biacore (Uppsala, Sweden) (Jönsson et al., Ann. Biol. Clin. 51 (1993), pp. 19-26), Windsor Scientific (U.K.) (URL for Windsor Scientific IBIS Biosensor), Quantech (Minnesota) (URL for Quantech), and Texas Instruments (Dallas, Tex.) (URL for Texas Instruments).

In yet another embodiment, a fluorescent polymer superquenching-based bioassays as disclosed in WO 02/074997 may be used for detecting binding of the unlabeled PET to its capture agents. In this embodiment, a capture agent that is specific for both a target PET peptide and a chemical moiety is used. The chemical moiety includes (a) a recognition element for the capture agent, (b) a fluorescent property-altering element, and (c) a tethering element linking the recognition element and the property-altering element. A composition comprising a fluorescent polymer and the capture agent are co-located on a support. When the chemical moiety is bound to the capture agent, the property-altering element of the chemical moiety is sufficiently close to the fluorescent polymer to alter (quench) the fluorescence emitted by the polymer. When an analyte sample is introduced, the target PET peptide, if present, binds to the capture agent, thereby displacing the chemical moiety from the receptor, resulting in de-quenching and an increase of detected fluorescence. Assays for detecting the presence of a target biological agent are also disclosed in the application.

In another related embodiment, the binding event between the capture agents and the PET can be detected by using a water-soluble luminescent quantum dot as described in US2003/0008414A1. In one embodiment, a water-soluble luminescent semiconductor quantum dot comprises a core, a cap and a hydrophilic attachment group. The “core” is a nanoparticle-sized semiconductor. While any core of the IIB-VIB, IIIB-VB or IVB-IVB semiconductors can be used in this context, the core must be such that, upon combination with a cap, a luminescent quantum dot results. A IIB-VIB semiconductor is a compound that contains at least one element from Group IEB and at least one element from Group VIB of the periodic table, and so on. Preferably, the core is a IIB-VIB, IIIB-VB or IVB-IVB semiconductor that ranges in size from about 1 nm to about 10 nm. The core is more preferably a IIB-VIB semiconductor and ranges in size from about 2 nm to about 5 nm. Most preferably, the core is CdS or CdSe. In this regard, CdSe is especially preferred as the core, in particular at a size of about 4.2 nm.

The “cap” is a semiconductor that differs from the semiconductor of the core and binds to the core, thereby forming a surface layer on the core. The cap must be such that, upon combination with a given semiconductor core, results in a luminescent quantum dot. The cap should passivate the core by having a higher band gap than the core. In this regard, the cap is preferably a IIB-VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe.

The “attachment group” as that term is used herein refers to any organic group that can be attached, such as by any stable physical or chemical association, to the surface of the cap of the luminescent semiconductor quantum dot and can render the quantum dot water-soluble without rendering the quantum dot no longer luminescent. Accordingly, the attachment group comprises a hydrophilic moiety. Preferably, the attachment group enables the hydrophilic quantum dot to remain in solution for at least about one hour, one day, one week, or one month. Desirably, the attachment group is attached to the cap by covalent bonding and is attached to the cap in such a manner that the hydrophilic moiety is exposed. Preferably, the hydrophilic attachment group is attached to the quantum dot via a sulfur atom. More preferably, the hydrophilic attachment group is an organic group comprising a sulfur atom and at least one hydrophilic attachment group. Suitable hydrophilic attachment groups include, for example, a carboxylic acid or salt thereof, a sulfonic acid or salt thereof, a sulfamic acid or salt thereof, an amino substituent, a quaternary ammonium salt, and a hydroxy. The organic group of the hydrophilic attachment group of the present invention is preferably a C1-C6 alkyl group or an aryl group, more preferably a C1-C6 alkyl group, even more preferably a C1-C3 alkyl group. Therefore, in a preferred embodiment, the attachment group of the present invention is a thiol carboxylic acid or thiol alcohol. More preferably, the attachment group is a thiol carboxylic acid. Most preferably, the attachment group is mercaptoacetic acid.

Accordingly, a preferred embodiment of a water-soluble luminescent semiconductor quantum dot is one that comprises a CdSe core of about 4.2 nm in size, a ZnS cap and an attachment group. Another preferred embodiment of a watersoluble luminescent semiconductor quantum dot is one that comprises a CdSe core, a ZnS cap and the attachment group mercaptoacetic acid. An especially preferred water-soluble luminescent semiconductor quantum dot comprises a CdSe core of about 4.2 nm, a ZnS cap of about 1 nm and a mercaptoacetic acid attachment group.

The capture agent of the instant invention can be attached to the quantum dot via the hydrophilic attachment group and forms a conjugate. The capture agent can be attached, such as by any stable physical or chemical association, to the hydrophilic attachment group of the water-soluble luminescent quantum dot directly or indirectly by any suitable means, through one or more covalent bonds, via an optional linker that does not impair the function of the capture agent or the quantum dot. For example, if the attachment group is mercaptoacetic acid and a nucleic acid biomolecule is being attached to the attachment group, the linker preferably is a primary amine, a thiol, streptavidin, neutravidin, biotin, or a like molecule. If the attachment group is mercaptoacetic acid and a protein biomolecule or a fragment thereof is being attached to the attachment group, the linker preferably is strepavidin, neutravidin, biotin, or a like molecule.

By using the quantum dot-capture agent conjugate, a PET-containing sample, when contacted with a conjugate as described above, will promote the emission of luminescence when the capture agent of the conjugate specifically binds to the PET peptide. This is particularly useful when the capture agent is a nucleic acid aptamer or an antibody. When the aptamer is used, an alternative embodiment may be employed, in which a fluorescent quencher may be positioned adjacent to the quantum dot via a self-pairing stem-loop structure when the aptamer is not bound to a PET-containing sequence. When the aptamer binds to the PET, the stem-loop structure is opened, thus releasing the quenching effect and generates luminescence.

In another related embodiment, arrays of nanosensors comprising nanowires or nanotubes as described in US2002/0117659A1 may be used for detection and/or quantitation of PET-capture agent interaction. Briefly, a “nanowire” is an elongated nanoscale semiconductor, which can have a cross-sectional dimension of as thin as 1 nanometer. Similarly, a “nanotube” is a nanowire that has a hollowed-out core, and includes those nanotubes know to those of ordinary skill in the art. A “wire” refers to any material having a conductivity at least that of a semiconductor or metal. These nanowires/nanotubes may be used in a system constructed and arranged to determine an analyte (e.g., PET peptide) in a sample to which the nanowire(s) is exposed. The surface of the nanowire is functionalized by coating with a capture agent. Binding of an analyte to the functionalized nanowire causes a detectable change in electrical conductivity of the nanowire or optical properties. Thus, presence of the analyte can be determined by determining a change in a characteristic in the nanowire, typically an electrical characteristic or an optical characteristic. A variety of biomolecular entities can be used for coating, including, but not limited to, amino acids, proteins, sugars, DNA, antibodies, antigens, and enzymes, etc. For more details such as construction of nanowires, functionalization with various biomolecules (such as the capture agents of the instant invention), and detection in nanowire devices, see US2002/0117659A1. Since multiple nanowires can be used in parelle, each with a different capture agent as the functionalized group, this technology is ideally suited for large scale arrayed detection of PET-containing peptides in biological samples without the need to label the PET peptides. This nanowire detection technology has been successfully used to detect pH change (H⁺ binding), biotin-streptavidin binding, antibody-antigen binding, metal (Ca²⁺) binding with picomolar sensitivity and in real time (Cui et al., Science 293: 1289-1292).

Matrix-assisted laser desorption/ionization time-of-flight mass spectrometry (MALDI-TOF MS), uses a laser pulse to desorb proteins from the surface followed by mass spectrometry to identify the molecular weights of the proteins (Gilligan et al., Mass spectrometry after capture and small-volume elution of analyte from a surface plasmon resonance biosensor. Anal. Chem. 74 (2002), pp. 2041-2047). Because this method only measures the mass of proteins at the interface, and because the desorption protocol is sufficiently mild that it does not result in fragmentation, MALDI can provide straightforward useful information such as confirming the identity of the bound PET peptide, or any enzymatic modification of a PET peptide. For this matter, MALDI can be used to identify proteins that are bound to immobilized capture agents. An important technique for identifying bound proteins relies on treating the array (and the proteins that are selectively bound to the array) with proteases and then analyzing the resulting peptides to obtain sequence.

### 9. Use of Multiple PETs in Highly Accurate Functional Measurement of Proteins

In certain embodiments of the invention, it may be advantageous to produce two or more PETs for each protein/fragment of interest. For example, two PETs within the same exon may be used to raise two different first capture antibodies or two different second (detection) antibodies to offer redundant measurement. Part of the reason is that trypsin digestion (or any other protease treatment or chemical fragmentation methods described above) could be incomplete or biased for/against certain fragments. Similarly, recovery of fragmented polypeptides by PET-specific capture agents may occasionally be incomplete and/or biased. Therefore, there may be certain risks associated with using one specific PET-specific capture agent for measurement of a target polypeptide.

To overcome this potential problem, or at least to compensate for the above-described incomplete digestion/recovery problems, two or more PETs specific to the polypeptide of interest may be generated, and used on the same array of the instant invention, or used in the same set of competition assays to independently detect different PETs of the same polypeptide. The average measurement results obtained by using such redundant PET-specific capture agents should be much more accurate and reliable when compared to results obtained using single PET-specific capture agents.

On the other hand, certain proteins may have different forms within the same biological sample. For example, proteins may be post translationally modified on one or more specific positions. There are more than 100 different kinds of post translational modifications, with the most common ones being acetylation, amidation, deamidation, prenylation, formylation, glycosy-lation, hydroxylation, methylation, myristoylation, phosphorylation, ubiquitination, SUMOylation, NEDDylation, ribosylation and sulphation. For a specific type of modification, such as phosphorylation, a PET peptide phosphorylated at a site may not be recognized by a capture agent raised against the same but unphosphorylated PET peptide. Therefore, by comparing the result of a first capture agent specific for un-modified PET peptide of a target protein (which represents unmodified target protein), with the result of a second capture agent specific for another PET within the same target protein (which does not contain any phosphorylation sites and thus representing the total amount of the target protein), one can determine the percentage of phosphorylated target protein within said sample.

The same principle applies to all target proteins with different forms, including unprocessed/pre-form and processed/mature form in certain growth factors, cytokines, and proteases; alternative splicing forms; and all types of post translational modifications.

In certain embodiments, capture agents specific for different PETs of the same target protein need not be of the same category (e.g., one could be an antibody specific for PET1, the other could be non-antibody binding protein for PET2, etc.)

In other embodiments, the presence or absence of one or more PETs is indicative of certain functional states of the target protein. For example, some PETs may be only present in unprocessed forms of certain proteins (such as peptide hormones, growth factors, cytokines, etc.), but not present in the corresponding mature/processed forms of the same proteins. This usually arises from the situation where the processing site resides within the PETs. On the other hand, other PETs might be common to both processed and unprocessed forms (e.g., do not contain any processing sites). If both types of PETs are used in the same array, or in the same competition assay, the abundance and ratio of processed/unprocessed target protein can be assessed.

In other embodiments, due to the vastly improved overall accuracy of the measurement using multiple PET-specific capture agents, the invention is applicable to the detection of certain previously unsuitable biomarkers because they have low detectable level (such as 1-5 pM) which is easily obscured by background signals. For example, as described above, Punglia et al. (N. Engl. J. Med. 349(4): 335-42, July, 2003) indicated that, in the standard PSA-based screening for prostate cancer, if the threshold PSA value for undergoing biopsy were set at 4.1 ng per milliliter, 82 percent of cancers in younger men and 65 percent of cancers in older men would be missed. Thus a lower threshold level of PSA for recommending prostate biopsy, particularly in younger men, may improve the clinical value of the PSA test. However, at lower detection limits, background can become a significant issue. The sensitivity/selectivity of the multiple PET-specific capture agent assay can be used to reliably and accurately detect low levels of PSA.

Similarly, due to the increased accuracy of measurements, small changes in concentration are more easily and reliably detected. Thus, the same method can also be used for other proteins previously unrecognized as disease biomarkers, by monitoring very small changes of protein levels very accurately. “Small changes” refers to a change in concentration of no more than about 50%, 40%, 30%, 20%, 15%, 10%, 5%, 1% or less when comparing a disease sample with a normal/control sample.

Accuracy of a measurement is usually defined by the degree of variation among individual measurements when compared to the true value, which can be reasonably accurately represented by the mean value of multiple independent measurements. The more accurate a method is, the closer a random measurement will be as compared to the mean value. A x % accuracy measurement means that x % of the measurements will be within one standardized deviation of the mean value. The method of the invention is usually at least about 70% accurate, preferably 80%, 90% or more accurate.

Detection of the presence and amount of the captured PET-containing polypeptide fragments can be effectuated using any of the methods described above that are generally applicable for detecting/quantitating the binding event.

To reiterate, for example, for each primary capture agent on an array, a specific, detectable secondary capture agent might be generated to bind the PET-containing peptide to be captured by the primary capture agent. The secondary capture agent may be specific for a second PET sequence on the to be captured polypeptide analyte, or may be specific for a post translational modification (such as phosphorylation) present on the to-be-captured polypeptide analyte. To facilitate detection/quantitation, the secondary capture agent may be labeled by a detectable moiety selected from: an enzyme, a fluorescent label, a stainable dye, a chemilumninescent compound, a colloidal particle, a radioactive isotope, a near-infrared dye, a DNA dendrimer, a water-soluble quantum dot, a latex bead, a selenium particle, or a europium nanoparticle.

Alternatively, the captured PET-containing polypeptide analytes may be detected directly using mass spectrometry, colorimetric resonant reflection using a SWS or SRVD biosensor, surface plasmon resonance (SPR), interferometry, gravimetry, ellipsometry, an evanascent wave device, resonance light scattering, reflectometry, a fluorescent polymer superquenching-based bioassay, or arrays of nanosensors comprising nanowires or nanotubes.

Another aspect of the invention provides arrays comprising redundant capture agents specific for one or more target proteins within a sample. Such arrays are useful to carry out the methods described above (e.g. high accuracy functional measurement of the target proteins). In one embodiment, several different capture agents are arrayed to detect different PET-containing peptide fragment derived from the same target protein. In other embodiments, the array may be used to detect several different target proteins, at least some (but not all) of which may be detected more than once by using capture agents specific for different PETs of those target proteins.

Another aspect of the invention provides a composition comprising a plurality of capture agents, wherein each of said capture agents recognizes and interacts with one PET of a target protein. The composition can be used in an array format in an array device as described above.

### Examples

### Example 1

### Generation of Plural Peptide Fragments Containing Potential Post-Translational Modification Sites and Antibodies to Detect the Plural Fragments

The following example shows how a post translational pattern within one protein or within a plurality of proteins can be determined. The sequences of eleven proteins were analyzed to determine Lys-C cleavage sites and tyrosine sites (pY sites) known to be phosphorylated under certain cellular conditions, as shown in Table 3. The first column in the table identifies the proteins that were analyzed; the second column identifies tyrosine residues of interest on each respective protein; and the third column identifies respective protein fragments generated from Lys-C digestion that contain the pY sites (underlined Y's in third column).

TABLE 3
Identification of protein post translational modification sites, fragments
containing the sites, and epitopes on each fragment

| Protein | pY Site | Lys C Fragment |
|---|---|---|
| pI3Kp110d | Y485 | GELLNPTGTVRSNPNTDSAAALLICLPEVAPHPVYYPALEK |
| | | (SEQ ID NO: 31) |
| | Y524 | ILELGRHSECVHVTEEEQLQLREILERRGSGELYEHEK |
| | | (SEQ ID NO: 32) |
| PLCγ2 | Y753/759 | MRLRYPVTPELLERYNMERDINSLYDVSRMYVDPSEINPSMPQRTVK |
| | | (SEQ ID NO: 33) |
| | Y11973217 | NGYSEDIELASLLVFCEMRPVLESEEELYSSCRQLRRRQEELNNQLFL |
| | | YDTHQNLRNANRDALVK |
| | | (SEQ ID NO: 34) |
| Src | Y138 | GERLQIVNNTEGDWWLAHSLSTGQTGYIPSNYVAPSDSIQAEEWYFGK |
| | | (SEQ ID NO: 35) |
| | Y215 | LDSGGFYITSRTQFNSLQQLVAYYSK |
| | | (SEQ ID NO: 36) |
| Gab1 | Y285/307/317 | VSPSSTEADGELYVFNTPSGTSSVETQMRHVSISYDIPPTPGNTYQIP |
| | | RTFPEGTLGQTSK |
| | | (SEQ ID NO: 37) |
| | Y373 | LDTIPDIPPPRPPKPHPAHDRSPVETCSIPRTASDTDSSYCIPTAGMS |
| | | PSRSNTISTVDLNK |
| | | (SEQ ID NO: 38) |
| EGFR | Y1069/1092 | EDSFLQRYSSDPTGALTEDSIDDTFLPVPEYINQSVPK |
| | | (SEQ ID NO: 39) |
| | Y1110/1125 | RPAGSVQNPVYHNQPLNPAPSRDPHYQDPHSTAVGNPEYLNTVQPTCV |
| | | NSTFDSPAHWAQK |
| | | (SEQ ID NO: 40) |
| IRS1 | Y46 | RFFVLRAASEAGGPARLEYYENEK |
| | | (SEQ ID NO: 41) |
| | Y465 | SQSSSNCSNPISVPLRRHHLNNPPPSQVGLTRRSRTESITATSPASMV |
| | | GGKPGSFRVRASSDGEGTMSRPASVDGSPVSPSTNRTHAHRHRGSARL |
| | | HPPLNHSRSIPMPASRCSPSATSPVSLSSSSTSGHGSTSDCLFPRRSS |
| | | ASVSGSPSDGGFISSDEYGSSPCDFRSSFRSVTPDSLGHTPPARGEEE |
| | | LSNYICMGGK |
| | | (SEQ ID NO: 42) |
| | Y612 | TPSQSSVASIEEYTEMMPAYPPGGGSGGRLPGHRHSAFVPTRSYPEEG |
| | | LEMHPLERRGGHHRPDSSTLHTDDGYMPMSPGVAPVPSGRK |
| | | (SEQ ID NO: 43) |
| | Y662 | SVSAPQQIINPIRRHPQRVDPNGYMMMSPSGGCSPDIGGGPSSSSSSS |
| | | NAVPSGTSYGK |
| | | (SEQ ID NO: 44) |
| | Y896/941 | SPGEYVNIEFGSDQSGYLSGPVAFHSSPSVRCPSQLQPAPREEETGTE |
| | | EYMK |
| | | (SEQ ID NO: 45) |
| | Y989 | MDLGPGRRAAWQESTGVEMGRLGPAPPGAASICRPTRAVPSSRGDYMT |
| | | MQMSCPRQSYVDTSPAAPVSYADMRTGIAAEEVSLPRATMAAASSSSA |
| | | ASASPTGPQGAAELAAHSSLLGGPQGPGGMSAFTRVNLSPNRNQSAK |
| | | (SEQ ID NO: 46) |
| IRS2 | Y540 | VALLPAGGALQHSRSMSMPVAHSPPAATSPGSLSSSSGHGSGSYPPPP |
| | | GPHPPLPHPLHHGPGQRPSSGSASASGSPSDPGFMSLDEYGSSPGDLR |
| | | AFCSHRSNTPESIAETPPARDGGGGGEFYGYMTMDRPLSHCGRSYRRV |
| | | SGDAAQDLDRGLRK |
| | | (SEQ ID NO: 47) |
| | Y625/628/653/ | VAYHPYPEDYGDIEIGSHRSSSSNLGADDGYMPMTPGAALAGSGSGSC |
| | 675 | RSDDYMPMSPASVSAPK |
| | | (SEQ ID NO: 48) |
| | Y823 | APYTCGGDSDQYVLMSSPVGRILEEERLEPQATPGPSQAASAFGAGPT |
| | | QPPHPVVPSPVRPSGGRPEGFLGQRGRAVRPTRLSLEGLPSLPSMHEY |
| | | PLPPEPK |
| | | (SEQ ID NO: 49) |
| | Y919 | SPGEYINIDFGEPGARLSPPAPPLLASAASSSSLLSASSPASSLGSGT |
| | | PGTSSDSRQRSPLSDYMNLDFSSPK |
| | | (SEQ ID NO: 50) |
| | Y1253 | SSEGGVGVGPGGGDEPPTSPRQLQPAPPLAPQGRPWTPGQPGGLVGCP |
| | | GSGGSPMRRETSAGFQNGLNYIAIDVREEPGLPPQPQPPPPPLPQPGD |
| | | K |
| | | (SEQ ID NO: 51) |
| FAK | Y347 | IAGAPEPLTVTAPSLTIAENMADLIDGYCRLVNGTSQSFIIRPQK |
| | | (SEQ ID NO: 52) |
| | Y397/407 | QGMRTHAVSVSETDDYAEIIDEEDTYTMPSTRDYEIQRERIELGRCIG |
| | | EGQFGDVHQGIYMSPENPALAVAIK |
| | | (SEQ ID NO: 53) |
| | Y861 | EERFLKPDVRLSRGSIDREDGSLQGPIGNQHIYQPVGKPDPAAPPK |
| | | (SEQ ID NO: 54) |
| Ezrin | Y145 | SGYLSSERLIPQRVMDQHK |
| | | (SEQ ID NO: 55) |
| | Y477 | EELHLVMTAPPPPPPPVYEPVSYHVQESLQDEGAEPTGYSAELSSEGI |
| | | RDDRNEEK |
| | | (SEQ ID NO: 56) |
| HER2 | Y1112/1139 | GLQSLPTHDPSPLQRYSEDPTVPLPSETDGYVAPLTCSPQPEYVNQPD |
| | | VRPQPPSPREGPLPAARPAGATLERPK |
| | | (SEQ ID NO: 57) |
| | Y1196/1221/ | DVFAFGGAVENPEYLTPQGGAAPQPHPPPAFSPAFDNLYYWDQDPPER |
| | 1222 | GAPPSTFK |
| | | (SEQ ID NO: 58) |
| LAB | Y136 | GSRHGSEEAYIDPIAMEYYNWGRFSKPPEDDDANSYENVLICK |
| | | (SEQ ID NO: 59) |
| | Y193 | TGPTSGLCPSASPEEDEESEDYQNSASIHQWRESRK |
| | | (SEQ ID NO: 60) |
| | Y233 | VMGQLQREASPGPVGSPDEEDGEPDYVNGEVAATEA |
| | | (SEQ ID NO: 61) |

The underlined multiple sequences in the third column of the table represent, on each fragment, an epitope that an antibody can be generated against. Specifically, using methods described above, antibodies to several of these peptide fragments across these several proteins were generated in which each fragment incorporated a tyrosine site that is known to be phosphorylated under certain cellular conditions.

As shown in the table, a target protein can have more than one potential post translational modification site. For example, the IRS2 protein has at least eight tyrosine residues that are potentially phosphorylated. The pY site or sites on each peptide can be detected with a common pY antibody. Experiments were conducted to verify that a capture antibody (that binds to an underlined epitope in the table) and pY detection antibody can form a sandwich with a synthetic version of the peptide fragment that contains the underlined sequence and the amino acid sequence around the pY site). Where more than one pY site is present on a particular fragment, a complementary fragmentation protocol can be used to produce different fragments that isolate each pY site. In this way, the state of each of the potential post translational modification sites within a protein or within a plurality of proteins can be identified to yield a highly resolved pattern of post translational modification.

### Example 2

### Detection and Quantitation of Phosphorylation at Specific Sites within the EGFR Protein in Response to Epidermal Growth Factor Activation in A431 Human Cancer Cells

The present invention allows for the study of site specific phosphorylation events within a protein or within a plurality of proteins using a sandwich assay. Since dysregulation of the EGFR signaling pathway has been observed in cells from many human disease states, a large emphasis in drug development has been placed on inhibiting aberrant EGFR signaling. However, there are multiple sites of phosphorylation that may be affected and the field would clearly benefit from a better understanding of any differential events that occur at particular phosphosites involved in response to different stimuli. This example describes a multiplexed sandwich immunoassay capable of detecting and quantitating a post translational modifications at specific sites within an EGFR protein.

Materials and Methods

A. Cell Culture

A431 cells were maintained at 37° C. with 5% CO₂ in DMEM (Invitrogen, Carlsbad, Calif.) supplemented with 10% heat-inactivated fetal bovine serum (Hyclone, Logan, Utah) and serum-starved for 24 hours when they reached 80% confluence. Media was removed and replaced with serum-free media containing 50 ng/mL EGF (Invitrogen) for various time points. Whole cell lysates were prepared in 5 mM TCEP (Pierce, Rockford, Ill.)/0.005% SDS (Sigma Aldrich, St. Louis, Mo.) lysis buffer containing freshly added 1 U/mL Benzonase (Novagen, San Diego, Calif.) and 1× Halt™ phosphatase inhibitor cocktail (Pierce). 15 μg of protein from whole cell lysates were resolved by SDS-PAGE and immunoblotted with anti-phosphotyrosine (Upstate, Billerica, Mass.) and anti-pERK1/2 antibodies (Biosource, Camarillo, Calif.) to confirm tyrosine phosphorylation and activation of the EGFR signaling pathway, respectively.

B. Sample Preparation

Whole cell lysates were boiled in TCEP/SDS lysis buffer for 5 minutes to reduce the samples, followed by alkylation with iodoacetamide (Sigma Aldrich) for 30 minutes in the dark. The arginine-specific endoproteinase, Lys-C (Wako, Richmond, Va.), was used to digest the proteins and digestion efficiency was evaluated by resolving proteins by SDS-PAGE.

C. Multiplex, Sandwich Immunoassay

Site-specificity of phosphotyrosine sites on the fragmented proteins was provided by capture antibodies designed to recognize specific amino acid sequences neighboring the phosphosite(s) of interest on each protein fragment. Each anti-peptide antibody was printed, in triplicate, onto Nexterion H slides (Schott, Elmsford, N.Y.) using a PeizoArray non-contact microarray system (Perkin Elmer, Waltham, Mass.) and blocked and washed according to the manufacturer's recommended protocols. After washing, Lys-C digested whole cell lysates from cells stimulated for different lengths of time with EGF were incubated for 1 hour on the slides followed by additional washing and incubation with a HiLyte-647 (Anaspec, San Jose, Calif.)-labeled anti-phosphotyrosine detection antibody (Upstate). Flourescence intensity was measured by the ScanArray Express HT slide reader (Perkin Elmer). The concentrations of total EGFR and site-specific, phospho-EGFR were determined by interpolation from standard curves generated with synthetic peptide standards.

### Results

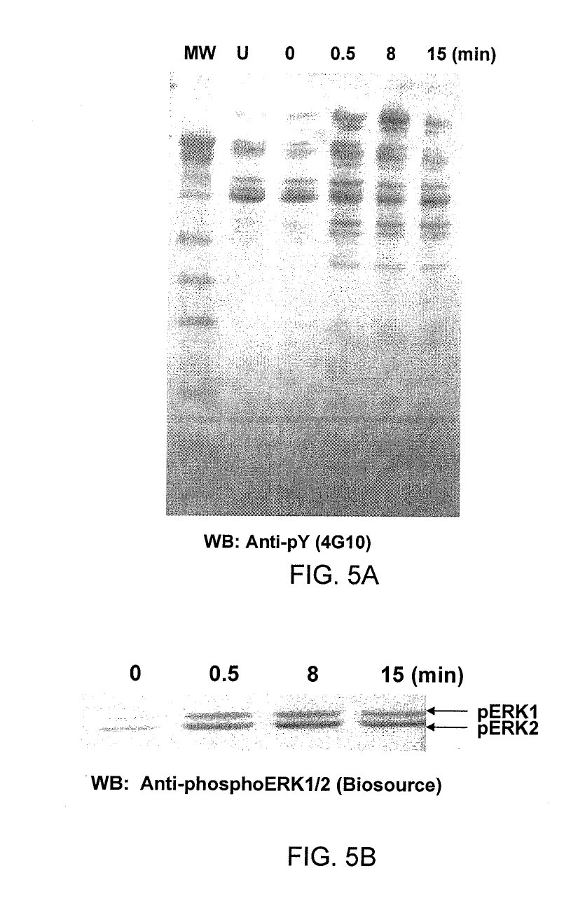

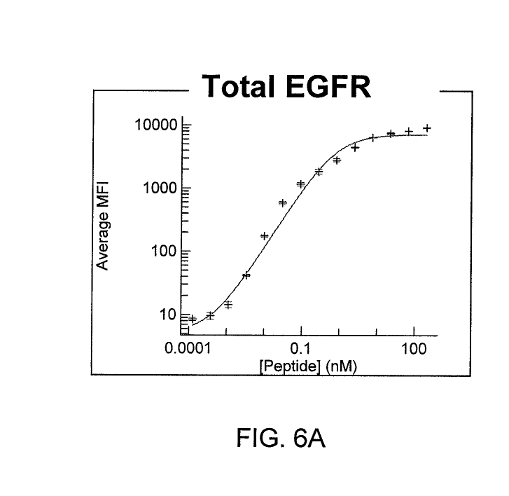

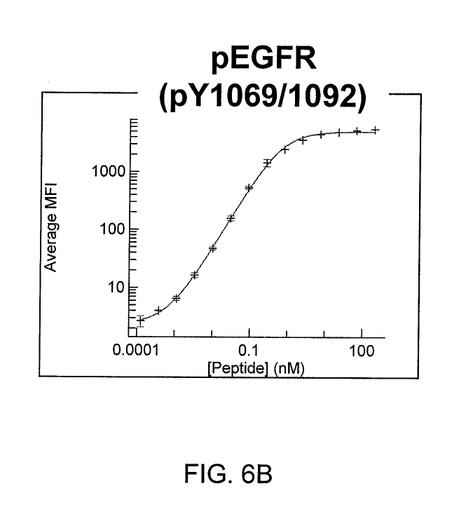

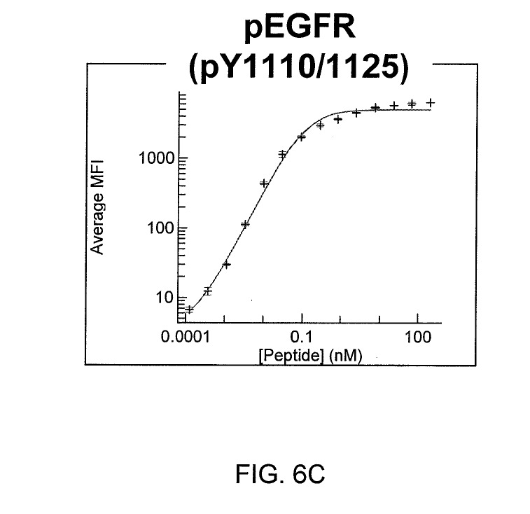

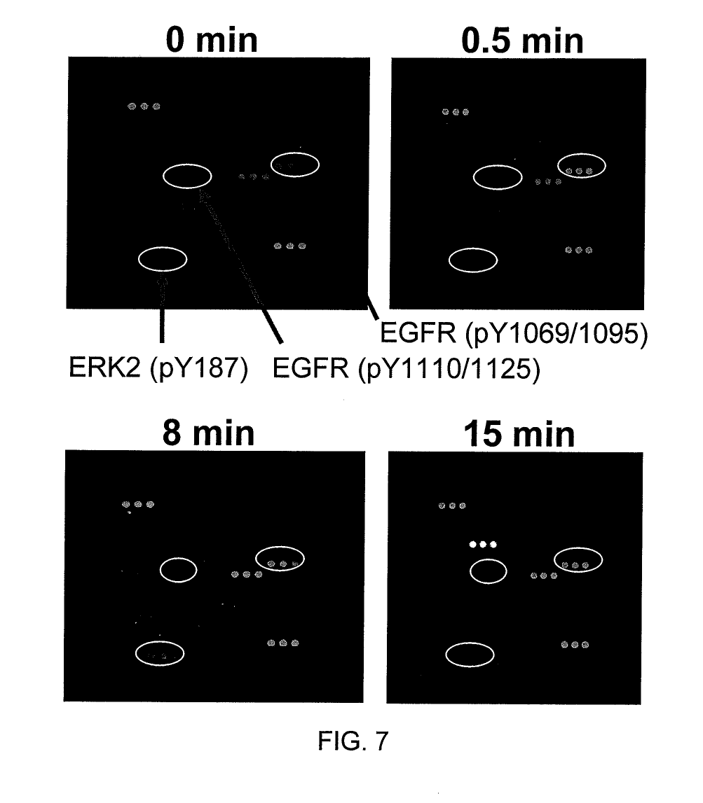

Increased tyrosine phosphorylation and activation of ERK1/2 in A431 cells was observed by 30 seconds exposure to 50 ng/mL EGF in Western blots of undigested cell lysates, as shown in FIG. 5A and FIG. 5B. Concentrations of phosphoproteins were determined by interpolation from synthetic peptide standard curves, as shown in FIGS. 6A-6C. Results from the multiplexed antibody array correlated with the results from the Western blots. As shown in FIG. 7, the phosphorylation level of ERK2 on tyrosine residue 187 was detected at 0.5 minutes following cellular exposure to EGF, and reached peak levels around 8 minutes of exposure to EGF. The same multiplexed assay detected increased phosphorylation on EGFR tyrosine residues 1069/1092 and tyrosine residues 1110/1125 in response to EGF stimulation, thereby showing an empirically consistent pattern across the post translational modification sites that were analyzed on the EGFR protein.

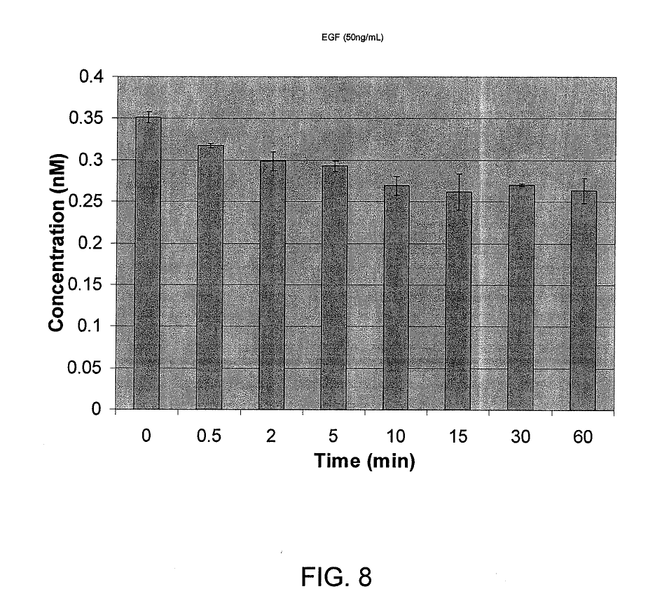

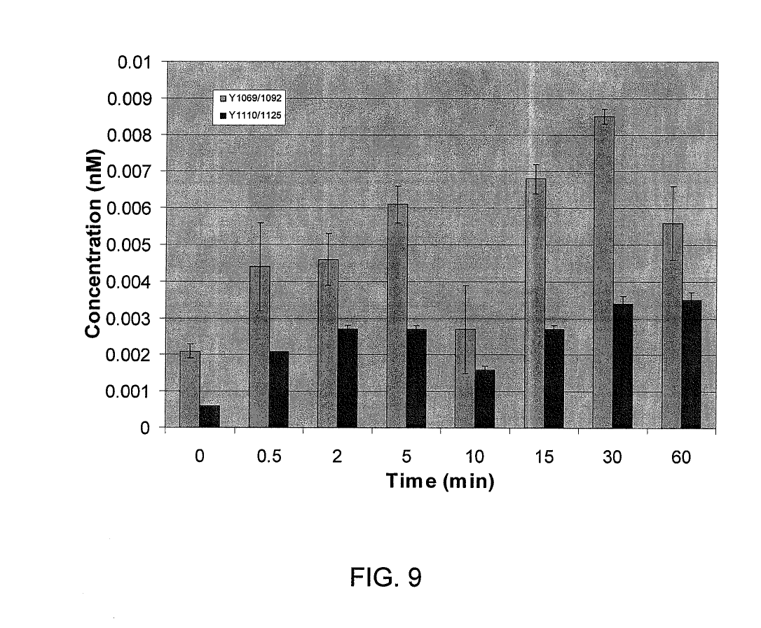

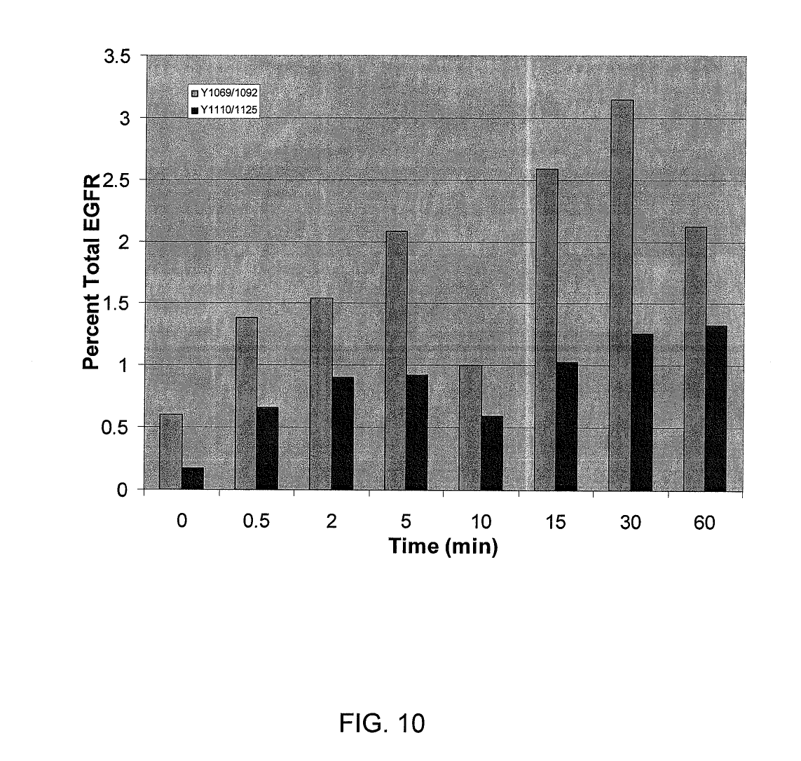

The concentrations of EGFR and the phosphorylated post translational modification sites on the EGFR protein were determined as a function of cellular exposure to EGF, using standard curves as described above. As shown in FIG. 8, the concentration of total EGFR protein decreased slightly over the first ten minutes of EGF stimulation and then remained fairly constant. As shown in FIG. 9, the concentration of phosphorylated EGFR tyrosine residues 1069/1092 trended upward until 30 minutes following cellular exposure to EGF. The concentration of phosphorylated EGFR tyrosine residues 1110/1125 followed a similar trend, though the concentration was less and the observed increases in concentration were less, than those for EGFR tyrosine residues 1069/1092. FIG. 10 shows these same phosphorylation concentrations for tyrosine residues 1069/1092 and 1110/1125, expressed as a percent of total EGFR protein at each time point. Thus, the simultaneous quantification of phosphorylation at various phosphosites within a single protein in a protein mixture was determined.

### INCORPORATION BY REFERENCE

The entire disclosure of each of the publications and patent documents referred to herein is incorporated by reference in its entirety for all purposes to the same extent as if each individual publication or patent document were so individually denoted.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A method for determining a pattern of post translational modification of a target protein, the method comprising:
(a) digesting the target protein using a predetermined proteolysis protocol to produce plural peptide fragments of the target protein, at least a portion of which comprises one or a plurality of sites for post translational modification and present an epitope capturable by a binding agent,
b) contacting said plural peptide fragments with immobilized binding agents which bind respectively to said epitopes and leave exposed a product of post translational modification thereby to capture plural said respective peptide fragments; and
c) contacting said captured respective peptide fragments with one or a plurality of labeled reagents which bind specifically to the exposed product of the post translational modification to discern which of the peptide fragments have been post translationally modified.

2. The method of claim 1 wherein said binding agents are immobilized in an array at known positions to permit determination of which peptide is post translationally modified.

3. The method of claim 1 wherein the post translational modification is phosphorylation.

4. The method of claim 3 wherein the one or a plurality of labeled reagents comprise a reagent that binds with one or more phosphorylated tyrosines, threonines, or serines.

5. The method of claim 3 wherein the one or a plurality of labeled reagents comprise a reagent selected from the group consisting of a phospho-binding domain from a macromolecule, a phospho-binding protein domain, an antibody or a part thereof that recognizes an epitope comprising a phosphate group, a phospho-binding aptamer domain or a portion thereof, a catalytically inert kinase construct or a part thereof which binds a phosphate group, and a catalytically inert phosphatase enzyme or a part thereof having phosphate binding activity.

6. The method of claim 3 wherein a said reagent specifically binds phosphorylated tyrosines.

7. The method of claim 3 wherein a said reagent specifically binds phosphorylated serines and/or threonines.

8. The method of claim 1 wherein multiple different proteins are digested in step a), and plural peptides from different proteins are captured in step b), thereby to enable the post translational modification pattern of multiple proteins to be determined simultaneously.

9. The method of claim 1 wherein said reagents are optically labeled.

10. The method of claim 1 wherein said target protein is present in a cell lysate that includes other proteins in addition to said target protein.

11. The method of claim 1 wherein the post translational modification is a modification selected from the group consisting of acetylation, amidation, deamidation, prenylation (such as farnesylation or geranylation), formylation, glycosylation, hydroxylation, methylation, myristoylation, phosphorylation, ubiquitination, SUMOylation, NEDDylation, ribosylation and sulphation.

12. The method of claim 1 further comprising quantitating the binding of the respective peptide fragments to the binding agents, or quantitating the binding of the reagent(s) to the captured respective peptide fragments to determine at least the relative quantity of different post translationally modified groups in different positions on the target protein(s), or the relative quantity of different differentially post translationally modified said target protein(s) in the sample.

13. The method of claim 2 wherein said labeled reagent is optically labeled, comprising detecting optical signals generated by said optical labels on a said reagent bound to a said respective peptide fragment captured at one or more selected positions on said array so that a pattern of signals on said array corresponds to a pattern of post translational modification on one or multiple said target proteins.

14. The method of claim 1 comprising the additional step of computationally deconvoluting data indicative of which of said fragments are modified and which fragments are not to determine the state of modification of one or more said target proteins.

15. An article of manufacture for determining a pattern of post translational modification on one or a plurality of target proteins in a sample, the apparatus comprising a set of immobilized capture agents, individual ones of which bind to peptide fragments generated by a predetermined digestion protocol applied to said one or plurality of target proteins, and which generated respective peptide fragments which:
(a) comprise an amino acid sequence which, when bound to a said capture agent, is an indication of the presence of said peptide fragment in the sample,
(b) comprise one or a plurality of sites for post translational modification, and,
(c) expose while bound to its capture agent a product of a post translational modification, if present on said fragment.

16. The article of claim 15 further comprising one or a plurality of detectably labeled reagent which bind specifically with said product of post translational modification exposed on said captured peptide fragments.

17. The article of claim 16 wherein said reagent binds specifically with said product of post translational modification thereby to reveal the pattern of post translational modification on respective individual said target proteins by deconvoluting the pattern of signals from said reagent labels bound to peptide fragments in turn bound to said set of immobilized capture agents.

18. The article of claim 17 wherein said reagent which binds specifically with said product of post translational modification binds with one or more phosphorylated tyrosines, phosphorylated threonines, or phosphorylated serines thereby to reveal the phosphorylation pattern of respective individual said target proteins.

19. The article of claim 16 wherein said reagent which binds specifically with said product of post translational modification binds with a product of post translational modification selected from the group consisting of acetylation, amidation, deamidation, prenylation (such as farnesylation or geranylation), formylation, glycosylation, hydroxylation, methylation, myristoylation, phosphorylation, ubiquitination, SUMOylation, NEDDylation, ribosylation and sulphation.

20. The article of claim 15 wherein the capture agents are immobilized on a solid surface at known positions in an array.

21. The article of claim 16 wherein said detectably labeled reagent comprises an optically detectable label.

22. The article of claim 15 further comprising a readable protocol specifying directions for digesting said target protein reliably to produce said peptide fragments.

23. The article of claim 15 further comprising apparatus or reagents for digesting said mixture of proteins in said sample reliably to produce said peptide fragments.

24. The article of claim 15 for obtaining the pattern of post translational modification of a plurality of target proteins in parallel, the apparatus further comprising a set of immobilized capture agents, individual ones of which bind to peptide fragments generated by a predetermined digestion protocol applied to said plurality of different target proteins, which generated fragments respectively comprise an amino acid sequence which, when bound to a said capture agent, is an indication of the presence of said peptide fragment in the sample, and permits resolution of different said target proteins in the sample.

25. A method of comparing a pattern of phosphorylation or other post translational modification of a target protein in plural cellular samples, the method comprising:
obtaining a first pattern of post translational modification of the target protein in a cellular sample under a first condition using the method of claim 1;
obtaining a second pattern of post translational modification of the target protein in a cellular sample under a second condition;
comparing the changes, if any, in pattern of post translational modification of the target protein between cells from the first and the second conditions.

26. The method of claim 25 wherein the first and second patterns are phosphorylation patterns.

27. The method of claim 25 wherein the first condition represents a first time point of the cellular sample, and the second condition represents a second, later time point of the cellular sample.

28. The method of claim 25 wherein the first condition of said cellular sample is before a stimulus and the second condition of said cellular sample represents the sample after the stimulus.

29. The method of claim 25 wherein the stimulus is exposure of the cells to a drug or drug candidate.

30. The method of claim 25 wherein the stimulus stimulates a cell surface receptor and said comparison determines the intracellular modifications of the intracellular portion of said receptor.

31. The method of claim 25, wherein the first condition represents cells from a healthy tissue, and the second condition represents cells from a diseased said tissue.


SEQUENCE LISTING (62 sequences)

SEQ ID NO: 1 - protein, 46 aa, Homo sapiens
NTSSSPQPKKKPLDGEYFTLQIRGRERFEMFRELNEALELKDAQAG

SEQ ID NO: 2 - protein, 36 aa, Homo sapiens
KPLDGEYFTLQIRGRERFEMFRELNEALELKDAQAG

SEQ ID NO: 3 - protein, 13 aa, Homo sapiens
ESTLHLVLRLRGG

SEQ ID NO: 4 - protein, 28 aa, Homo sapiens
QLEDGRTLSDYNIQKESTLHLVLRLRGG

SEQ ID NO: 5 - protein, 11 aa, Unknown
  Description of Unknown Phosphorylation site for cAMP-dependent Protein Kinase
YLRRASLAQLT

SEQ ID NO: 6 - protein, 8 aa, Unknown
  Description of Unknown Phosphorylation site for cAMP-dependent Protein Kinase
FRRLSIST

SEQ ID NO: 7 - protein, 11 aa, Unknown
  Description of Unknown Phosphorylation site for cAMP-dependent Protein Kinase
AGARRKASGPP

SEQ ID NO: 8 - protein, 11 aa, Unknown
  Description of Unknown Phosphorylation site for Casein Kinase I
RTLSVSSLPGL

SEQ ID NO: 9 - protein, 10 aa, Unknown
  Description of Unknown Phosphorylation site for Casein Kinase I
DIGSESTEDQ

SEQ ID NO: 10 - protein, 10 aa, Unknown
  Description of Unknown Phosphorylation site for Casein Kinase II
ADSESEDEED

SEQ ID NO: 11 - protein, 11 aa, Unknown
  Description of Unknown Phosphorylation site for Casein Kinase II
LESEEEGVPST

SEQ ID NO: 12 - protein, 11 aa, Unknown
  Description of Unknown Phosphorylation site for Casein Kinase II
EDNSEDEISNL

SEQ ID NO: 13 - protein, 9 aa, Unknown
  Description of Unknown Phosphorylation site for Glycogen Synthase Kinase 3
SVPPSPSLS

SEQ ID NO: 14 - protein, 9 aa, Unknown
  Description of Unknown Phosphorylation site for Glycogen Synthase Kinase 3
SVPPSPSLS

SEQ ID NO: 15 - protein, 7 aa, Unknown
  Description of Unknown Phosphorylation site for Cdc2 Protein Kinase
PAKTPVK

SEQ ID NO: 16 - protein, 10 aa, Unknown
  Description of Unknown Phosphorylation site for Cdc2 Protein Kinase
HSTPPKKKRK

SEQ ID NO: 17 - protein, 11 aa, Unknown
  Description of Unknown Phosphorylation site for Calmodulin-dependent Protein Kinase II
NYLRRRLSDSN

SEQ ID NO: 18 - protein, 10 aa, Unknown
  Description of Unknown Phosphorylation site for Calmodulin-dependent Protein Kinase II
KMARVFSVLR

SEQ ID NO: 19 - protein, 4 aa, Unknown
  Description of Unknown Phosphorylation site for Mitogen-activated Protein Kinase
PLSP

SEQ ID NO: 20 - protein, 4 aa, Unknown
  Description of Unknown Phosphorylation site for Mitogen-activated Protein Kinase
PSSP

SEQ ID NO: 21 - protein, 4 aa, Unknown
  Description of Unknown Phosphorylation site for Mitogen-activated Protein Kinase
VLSP

SEQ ID NO: 22 - protein, 11 aa, Unknown
  Description of Unknown Phosphorylation site for cGMP-dependent Protein Kinase
GKKRKRSRKES

SEQ ID NO: 23 - protein, 8 aa, Unknown
  Description of Unknown Phosphorylation site for cGMP-dependent Protein Kinase
FRRLSIST

SEQ ID NO: 24 - protein, 12 aa, Unknown
  Description of Unknown Phosphorylation site for Phosphorylase Kinase
DQEKRKQISVRG

SEQ ID NO: 25 - protein, 10 aa, Unknown
  Description of Unknown Phosphorylation site for Phosphorylase Kinase
PLSRTLSVSS

SEQ ID NO: 26 - protein, 9 aa, Unknown
  Description of Unknown Phosphorylation site for Protein Kinase C
HEGTHSTKR

SEQ ID NO: 27 - protein, 10 aa, Unknown
  Description of Unknown Phosphorylation site for Protein Kinase C
PLSRTLSVSS

SEQ ID NO: 28 - protein, 11 aa, Unknown
  Description of Unknown Phosphorylation site for Protein Kinase C
QKRPSQRSKYL

SEQ ID NO: 29 - protein, 11 aa, Unknown
  Description of Unknown Phosphorylation site for EpidermalGrowth Factor Receptor Kinase
RENAEYLRVAP

SEQ ID NO: 30 - protein, 10 aa, Unknown
  Description of Unknown Phosphorylation site for EpidermalGrowth Factor Receptor Kinase
AEPDYGALYE

SEQ ID NO: 31 - protein, 41 aa, Homo sapiens
GELLNPTGTVRSNPNTDSAAALLICLPEVAPHPVYYPALEK

SEQ ID NO: 32 - protein, 38 aa, Homo sapiens
ILELGRHSECVHVTEEEQLQLREILERRGSGELYEHEK

SEQ ID NO: 33 - protein, 47 aa, Homo sapiens
MRLRYPVTPELLERYNMERDINSLYDVSRMYVDPSEINPSMPQRTVK

SEQ ID NO: 34 - protein, 65 aa, Homo sapiens
NGYSEDIELASLLVFCEMRPVLESEEELYSSCRQLRRRQEELNNQLFLYDTHQNLRNANRDALVK

SEQ ID NO: 35 - protein, 48 aa, Homo sapiens
GERLQIVNNTEGDWWLAHSLSTGQTGYIPSNYVAPSDSIQAEEWYFGK

SEQ ID NO: 36 - protein, 26 aa, Homo sapiens
LDSGGFYITSRTQFNSLQQLVAYYSK

SEQ ID NO: 37 - protein, 61 aa, Homo sapiens
VSPSSTEADGELYVFNTPSGTSSVETQMRHVSISYDIPPTPGNTYQIPRTFPEGTLGQTSK

SEQ ID NO: 38 - protein, 62 aa, Homo sapiens
LDTIPDIPPPRPPKPHPAHDRSPVETCSIPRTASDTDSSYCIPTAGMSPSRSNTISTVDLNK

SEQ ID NO: 39 - protein, 38 aa, Homo sapiens
EDSFLQRYSSDPTGALTEDSIDDTFLPVPEYINQSVPK

SEQ ID NO: 40 - protein, 61 aa, Homo sapiens
RPAGSVQNPVYHNQPLNPAPSRDPHYQDPHSTAVGNPEYLNTVQPTCVNSTFDSPAHWAQK

SEQ ID NO: 41 - protein, 24 aa, Homo sapiens
RFFVLRAASEAGGPARLEYYENEK

SEQ ID NO: 42 - protein, 202 aa, Homo sapiens
SQSSSNCSNPISVPLRRHHLNNPPPSQVGLTRRSRTESITATSPASMVGGKPGSFRVRASSDGEGTMSRPASVDGSPVSPSTNRTHAHRHRGSARLHPPLNHSRSIPMPASRCSPSATSPVSLSSSSTSGHGSTSDCLFPRRSSASVSGSPSDGGFISSDEYGSSPCDFRSSFRSVTPDSLGHTPPARGEEELSNYICMGGK

SEQ ID NO: 43 - protein, 89 aa, Homo sapiens
TPSQSSVASIEEYTEMMPAYPPGGGSGGRLPGHRHSAFVPTRSYPEEGLEMHPLERRGGHHRPDSSTLHTDDGYMPMSPGVAPVPSGRK

SEQ ID NO: 44 - protein, 59 aa, Homo sapiens
SVSAPQQIINPIRRHPQRVDPNGYMMMSPSGGCSPDIGGGPSSSSSSSNAVPSGTSYGK

SEQ ID NO: 45 - protein, 52 aa, Homo sapiens
SPGEYVNIEFGSDQSGYLSGPVAFHSSPSVRCPSQLQPAPREEETGTEEYMK

SEQ ID NO: 46 - protein, 143 aa, Homo sapiens
MDLGPGRRAAWQESTGVEMGRLGPAPPGAASICRPTRAVPSSRGDYMTMQMSCPRQSYVDTSPAAPVSYADMRTGIAAEEVSLPRATMAAASSSSAASASPTGPQGAAELAAHSSLLGGPQGPGGMSAFTRVNLSPNRNQSAK

SEQ ID NO: 47 - protein, 158 aa, Homo sapiens
VALLPAGGALQHSRSMSMPVAHSPPAATSPGSLSSSSGHGSGSYPPPPGPHPPLPHPLHHGPGQRPSSGSASASGSPSDPGFMSLDEYGSSPGDLRAFCSHRSNTPESIAETPPARDGGGGGEFYGYMTMDRPLSHCGRSYRRVSGDAAQDLDRGLRK

SEQ ID NO: 48 - protein, 65 aa, Homo sapiens
VAYHPYPEDYGDIEIGSHRSSSSNLGADDGYMPMTPGAALAGSGSGSCRSDDYMPMSPASVSAPK

SEQ ID NO: 49 - protein, 103 aa, Homo sapiens
APYTCGGDSDQYVLMSSPVGRILEEERLEPQATPGPSQAASAFGAGPTQPPHPVVPSPVRPSGGRPEGFLGQRGRAVRPTRLSLEGLPSLPSMHEYPLPPEPK

SEQ ID NO: 50 - protein, 73 aa, Homo sapiens
SPGEYINIDFGEPGARLSPPAPPLLASAASSSSLLSASSPASSLGSGTPGTSSDSRQRSPLSDYMNLDFSSPK

SEQ ID NO: 51 - protein, 97 aa, Homo sapiens
SSEGGVGVGPGGGDEPPTSPRQLQPAPPLAPQGRPWTPGQPGGLVGCPGSGGSPMRRETSAGFQNGLNYIAIDVREEPGLPPQPQPPPPPLPQPGDK

SEQ ID NO: 52 - protein, 45 aa, Homo sapiens
IAGAPEPLTVTAPSLTIAENMADLIDGYCRLVNGTSQSFIIRPQK

SEQ ID NO: 53 - protein, 73 aa, Homo sapiens
QGMRTHAVSVSETDDYAEIIDEEDTYTMPSTRDYEIQRERIELGRCIGEGQFGDVHQGIYMSPENPALAVAIK

SEQ ID NO: 54 - protein, 46 aa, Homo sapiens
EERFLKPDVRLSRGSIDREDGSLQGPIGNQHIYQPVGKPDPAAPPK

SEQ ID NO: 55 - protein, 19 aa, Homo sapiens
SGYLSSERLIPQRVMDQHK

SEQ ID NO: 56 - protein, 56 aa, Homo sapiens
EELHLVMTAPPPPPPPVYEPVSYHVQESLQDEGAEPTGYSAELSSEGIRDDRNEEK

SEQ ID NO: 57 - protein, 75 aa, Homo sapiens
GLQSLPTHDPSPLQRYSEDPTVPLPSETDGYVAPLTCSPQPEYVNQPDVRPQPPSPREGPLPAARPAGATLERPK

SEQ ID NO: 58 - protein, 56 aa, Homo sapiens
DVFAFGGAVENPEYLTPQGGAAPQPHPPPAFSPAFDNLYYWDQDPPERGAPPSTFK

SEQ ID NO: 59 - protein, 43 aa, Homo sapiens
GSRHGSEEAYIDPIAMEYYNWGRFSKPPEDDDANSYENVLICK

SEQ ID NO: 60 - protein, 36 aa, Homo sapiens
TGPTSGLCPSASPEEDEESEDYQNSASIHQWRESRK

SEQ ID NO: 61 - protein, 36 aa, Homo sapiens
VMGQLQREASPGPVGSPDEEDGEPDYVNGEVAATEA

SEQ ID NO: 62 - protein, 5 aa, Artificial Sequence
  Description of Artificial Sequence Synthetic peptide
GSSSS